SEQUENCE LISTING (52 sequences)

SEQ ID NO: 1 - protein, 133 aa, Homo Sapiens [sequence omitted]

SEQ ID NO: 2 - protein, 52 aa, Homo Sapiens [sequence omitted]

SEQ ID NO: 3 - DNA, 2032 bp, Homo Sapiens
gatactttagagcggagggatgtattgaaacagactactgctacttacagcaccgtatagcagccctgctcctacattttgctgccttactctgccccgaatgcactggagtggggatggtccatcggcaactataaactgattctcatcaggaaactgcacattatctccccatcacttcaaaggtctcgtcaggcagaggtgacgccaggagatgatttaaaggtgaaaatgacaaggtttccacccctcaaaccttggctccttttctgacaatacagtctgaatgaacccgatgtctttttttttactgtggaaataggatcggaagagagtaacatttttttttttttaatcctgataaagaagattgttgggaagctctttgaaaaaaaattttaaattgtggcacagatggattttaaaaagtgttagatctttccaatgaacactaatagagtactctgctcttggctggatttttcagagaatggcaatggtctctgcgatgtcctgggtcctgtatttgtggataagtgcttgtgcaatgctactctgccatggatcccttcagcacactttccagcagcatcacctgcacagaccagaaggagggacgtgtgaagtgatagcagcacaccgatgttgtaacaagaatcgcattgaggagcggtcacaaacagtaaagtgttcctgtctacctggaaaagtggctggaacaacaagaaaccggccttcttgcgtcgatgcctccatagtgattgggaaatggtggtgtgagatggagccttgcctagaaggagaagaatgtaagacactccctgacaattctggatggatgtgcgcaacaggcaacaaaattaagaccacgagaattcacccaagaacctaacagaagcatttgtggtagtaaaggaaaaccaaccctctggaaaatacattttgagaatctcaaacatctcacatatatacaagccaaatggatttcttacttgcactttgactggctaccagataatcacagtgcgtttactgtgtgtaacgaaatatcctacagtgagaagacacagcgttttggcaacaccatggaaagtgggcttaaaaaagggttttctcagtgaaatttttgggcatcatgaagaacgatcaactatcttctaatttgaatctatagttactttgtaccatttgaaatatatgtatatatatatatataatattttgaaatattatctattctcttcaagaaatgaacagtaccacagtttgagacggctggtgtacccctttgagttttggatgttttgtctgttttgctttgttttgttagtcatttctttttctaacggcaaggaagatatgtgcccttttgagaattcaagatggcactgacacgggaaggccagctacaggtggactcctggaatttgaggcatcataatgatactgaatcaagaacttccttctgcttctaccagatggcccaaggaagcacatcgtcctgttttattgctttctaccctgtgcaatattagcatgcaagcttggcttacatagtcatactttatattcaattgatatataataaccgttctaacctcttccaggaaaatatttttagaactactagcttttccacttagaagaaaatgaggattcttaagggagccactccaccatgctattaagactctggcagagttatgggtaggatatggatccctacatgaataagtcctgtaaatacaatgtcttaaggctttgtatagctgtcctagactgcagaaatgtcctctgattaaatccaaagtctggcatcgttaactacatagtgctgtagcaacaagtcttatcatggcatctctttctatgtttggtttgctttttccaagagtattcaggtctcctcttgtgagataggaaggccatgaaaacaattagatttcaagatgatctatgtgaccaaatgttggacagccctattaaagtggtaaacaacttctttctaaaaaaaaaaaaaaaaaaaaaaaaa

SEQ ID NO: 4 - protein, 8 aa, Artificial Sequence
  Anti-FAM19A1 (“1A11”) VH-CDR1
GYSFTGYD

SEQ ID NO: 5 - protein, 8 aa, Artificial Sequence
  Anti-FAM19A1 (“1A11”) VH-CDR2
MNPSSGNT

SEQ ID NO: 6 - protein, 15 aa, Artificial Sequence
  Anti-FAM19A1 (“1A11”) VH-CDR3
ARALNSVYYYHALDV

SEQ ID NO: 7 - protein, 6 aa, Artificial Sequence
  Anti-FAM19A1 (“1A11”) VL-CDR1
RLHNKY

SEQ ID NO: 8 - protein, 3 aa, Artificial Sequence
  Anti-FAM19A1 (“1A11”) VL-CDR2
QDA

SEQ ID NO: 9 - protein, 9 aa, Artificial Sequence
  Anti-FAM19A1 (“1A11”) VL-CDR3
QTWDRSTGV

SEQ ID NO: 10 - protein, 8 aa, Artificial Sequence
  Anti-FAM19A1 (“1C1”) VH-CDR1
GFAFSDYG

SEQ ID NO: 11 - protein, 8 aa, Artificial Sequence
  Anti-FAM19A1 (“1C1”) VH-CDR2
ISYDGSKR

SEQ ID NO: 12 - protein, 12 aa, Artificial Sequence
  Anti-FAM19A1 (“1C1”) VH-CDR3
ARPGDYALAFDL

SEQ ID NO: 13 - protein, 9 aa, Artificial Sequence
  Anti-FAM19A1 (“1C1”) VL-CDR1
SGSVSTSHF

SEQ ID NO: 14 - protein, 3 aa, Artificial Sequence
  Anti-FAM19A1 (“1C1”) VL-CDR2
SRN

SEQ ID NO: 15 - protein, 10 aa, Artificial Sequence
  Anti-FAM19A1 (“1C1”) VL-CDR3
ALYMGRGNWV

SEQ ID NO: 16 - protein, 8 aa, Artificial Sequence
  Anti-FAM19A1 (“2G7”) VH-CDR1
GFPFGDHA

SEQ ID NO: 17 - protein, 10 aa, Artificial Sequence
  Anti-FAM19A1 (“2G7”) VH-CDR2
IRSNTYGGTT

SEQ ID NO: 18 - protein, 22 aa, Artificial Sequence
  Anti-FAM19A1 (“2G7”) VH-CDR3
TKDITGGGFWSDYGDYFDAFDF

SEQ ID NO: 19 - protein, 6 aa, Artificial Sequence
  Anti-FAM19A1 (“2G7”) VL-CDR1
KLGYKY

SEQ ID NO: 20 - protein, 3 aa, Artificial Sequence
  Anti-FAM19A1 (“2G7”) VL-CDR2
QDK

SEQ ID NO: 21 - protein, 11 aa, Artificial Sequence
  Anti-FAM19A1 (“2G7”) VL-CDR3
QAWDSGTASHV

SEQ ID NO: 22 - protein, 10 aa, Artificial Sequence
  Anti-FAM19A1 (“3A8”) VH-CDR1
GDAITSGSYY

SEQ ID NO: 23 - protein, 7 aa, Artificial Sequence
  Anti-FAM19A1 (“3A8”) VH-CDR2
ISHSGST

SEQ ID NO: 24 - protein, 12 aa, Artificial Sequence
  Anti-FAM19A1 (“3A8”) VH-CDR3
AGDTALVGAYSI

SEQ ID NO: 25 - protein, 6 aa, Artificial Sequence
  Anti-FAM19A1 (“3A8”) VL-CDR1
NLRTKY

SEQ ID NO: 26 - protein, 3 aa, Artificial Sequence
  Anti-FAM19A1 (“3A8”) VL-CDR2
QDT

SEQ ID NO: 27 - protein, 11 aa, Artificial Sequence
  Anti-FAM19A1 (“3A8”) VL-CDR3
MTWDVDTTSMI

SEQ ID NO: 28 - protein, 122 aa, Artificial Sequence
  Anti-FAM19A1 (“1A11”) VH
QVQLVESGAEVRKPGASVKVSCKASGYSFTGYDINWVRQAPGQGLEWMGWMNPSSGNTGYAQKFQGRVTMTRDSSISTAYMELSSLRSEDTAVYYCARALNSVYYYHALDVWGQGTTITVSS

SEQ ID NO: 29 - protein, 106 aa, Artificial Sequence
  Anti-FAM19A1 (“1A11”) VL
SYELTQPPSASVSPGQTASITCSGDRLHNKYTSWYQQKPGQSPLLVIYQDAKRPSGIPERFSGSSSRGTATLTISGTQATDEADYYCQTWDRSTGVFGTGTKVTVL

SEQ ID NO: 30 - protein, 119 aa, Artificial Sequence
  Anti-FAM19A1 (“1C1”) VH
QVQLVESGGGVAQPGRSLRLSCAASGFAFSDYGIHWVRQAPGKGLEWVALISYDGSKRSYADSVKGRFAISRDNSKNTLYLQMNSLRAEDTAVYYCARPGDYALAFDLWGQGTMVTVSS

SEQ ID NO: 31 - protein, 110 aa, Artificial Sequence
  Anti-FAM19A1 (“1C1”) VL
QTVVTQEPSFSVSPGETVTLTCGLSSGSVSTSHFPSWYRQTPGQAPRPLIDSRNARSIGVPDRFSGSIVGTKATLTISGAQAEDECNYYCALYMGRGNWVFGGGTKLTVL

SEQ ID NO: 32 - protein, 131 aa, Artificial Sequence
  Anti-FAM19A1 (“2G7”) VH
QVQLVESGGGLVKPGRSLSLSCTTSGFPFGDHAINWVRQAPGKGLEWVGFIRSNTYGGTTQYAASVEGRFTISRDDSKSIAYLQMNSLRAEDTAVYYCTKDITGGGFWSDYGDYFDAFDFWGQGTMVTVSS

SEQ ID NO: 33 - protein, 108 aa, Artificial Sequence
  Anti-FAM19A1 (“2G7”) VL
SYELTQPLSVSVSPGQTASISCSGDKLGYKYASWYQQKPGQSPVVVIYQDKKRPSGIPERFSGSNSGNTATLTISGTQPMDEADYYCQAWDSGTASHVFGTGTKVTVL

SEQ ID NO: 34 - protein, 120 aa, Artificial Sequence
  Anti-FAM19A1 (“3A8”) VH
QVQLQQSGPGLVKPSETLSLICSVSGDAITSGSYYWGWIRQSPGRGLEWIGEISHSGSTDYNPSLKSRVTISVDKSRNQFSLRLNSVTAVDTAVYYCAGDTALVGAYSIWGQGTMVTVSS

SEQ ID NO: 35 - protein, 108 aa, Artificial Sequence
  Anti-FAM19A1 (“3A8”) VL
SYELTQAPSLSVSPGQTANIICSGDNLRTKYVSWYQQKPGQSPLLVIYQDTRRPSGIPARFSGSNSGNTATLTISGTQTRDESTYYCMTWDVDTTSMIFGGGTKLTVL

SEQ ID NO: 36 - DNA, 366 bp, Artificial Sequence
  Anti-FAM19A1 (“1A11”) VH
caggtgcagctggtggagtctggggctgaggtgaggaagcctggggcctcagtgaaggtctcctgcaaggcttctggatatagtttcaccggttatgacatcaactgggtgcgacaggcccctggacaagggcttgagtggatgggatggatgaaccccagtagtggtaatacaggctatgcacagaagtttcagggcagagtcaccatgaccagggacagctccataagcacagcctacatggagctgagcagcctgagatctgaggacacggccgtgtattactgtgcgagagctttaaattcggtctactactaccacgctctggacgtctggggccaagggaccacgatcaccgtctcctca

SEQ ID NO: 37 - DNA, 318 bp, Artificial Sequence
  Anti-FAM19A1 (“1A11”) VL
tcctatgagctgacacagccaccctcagcgtccgtgtccccaggacagacagccagcatcacctgctctggagatagattacacaataaatatacttcctggtatcaacagaagccaggccagtcccctctactggtcatctatcaagatgccaagcgaccctcagggatccctgagcgattctcgggctccagctctcggggcacagccactctgaccatcagcgggacccaggctacggatgaggctgactattactgtcaaacgtgggacaggagcactggagtcttcggaactgggaccaaggtcaccgtccta

SEQ ID NO: 38 - DNA, 357 bp, Artificial Sequence
  Anti-FAM19A1 (“1C1”) VH
caggtgcagctggtggagtcggggggaggcgtggcccagcctgggaggtccctgagactctcctgtgcagcctctggattcgccttcagtgactatggcatacactgggtccgccaggctccaggcaaggggctggagtgggtggcacttatatcatatgatggaagtaagagatcctatgcagactccgtgaagggccgattcgccatctccagagacaattccaagaacacgctgtatctgcaaatgaacagtctgagagccgaggacacggccgtgtattactgtgcgagaccgggcgattatgccctcgcttttgatctctggggccaagggacaatggtcaccgtctcctca

SEQ ID NO: 39 - DNA, 330 bp, Artificial Sequence
  Anti-FAM19A1 (“1C1”) VL
cagactgtggtgactcaggagccatcgttctcagtgtcccctggagagacagtcaccctcacttgtggcttgagctctggctcagtctctacttctcacttccccagttggtaccgacagactccaggccaggctccacgcccgctcatcgacagcagaaacgctcgctctattggggtccctgatcgcttctctggctccatcgttgggaccaaggctaccctgaccatctcgggggcccaggcagaagatgaatgtaattattactgtgctctgtatatgggtcgtggcaattgggtgttcggcggagggaccaagctgaccgtccta

SEQ ID NO: 40 - DNA, 393 bp, Artificial Sequence
  Anti-FAM19A1 (“2G7”) VH
caggtgcagctggtagagtctgggggaggcttggtaaagccagggcggtccctgagcctctcctgtacaacttctggattcccctttggtgatcatgccataaattgggtccgccaggctccagggaagggcctggagtgggttggtttcatcaggagtaacacttatggtgggacaacacagtacgccgcgtctgtggagggcagattcaccatctcaagagacgattccaaaagcatcgcctatctgcaaatgaacagtctgagagccgaggacacggccgtgtattactgtacaaaagatataaccgggggcgggttttggagcgactacggtgactactttgatgcttttgatttctggggccaagggacaatggtcaccgtctcctca

SEQ ID NO: 41 - DNA, 324 bp, Artificial Sequence
  Anti-FAM19A1 (“2G7”) VL
tcctatgagctgacacagccactctcagtgtccgtgtccccaggacagacagccagcatctcctgctctggggataaattgggttacaaatatgcttcctggtatcagcagaagccgggccagtcccctgtggtggtcatctatcaagataaaaagcggccctcagggatccctgagcgattctctggctccaactctgggaacacagccactctgaccatcagcgggacccagcctatggatgaggctgactattattgtcaggcgtgggacagcggcactgcctctcatgtcttcggaactgggaccaaggtcaccgtccta

SEQ ID NO: 42 - DNA, 360 bp, Artificial Sequence
  Anti-FAM19A1 (“3A8”) VH
caggtgcagctgcagcagtcgggcccaggactggtgaagccttcggagaccctgtccctcatctgcagtgtctctggtgacgccatcaccagtggttcttattactggggctggatccgccagtccccagggagggggctggagtggattggggaaatctctcatagtgggagcaccgactacaacccgtccctcaagagtcgagtcaccatatcagtagacaagtccaggaaccagttctctctgagactgaactctgtgaccgccgtggacacggccgtgtattactgtgcgggagatacagccttggtcggtgcttattctatctggggccaagggacaatggtcaccgtctcctca

SEQ ID NO: 43 - DNA, 324 bp, Artificial Sequence
  Anti-FAM19A1 (“3A8”) VL
tcctatgagctgacacaggcaccctcactgtccgtgtcgccaggacagacagccaacatcatctgctctggagataacttgcgtactaaatatgtttcttggtatcagcagaagccaggccagtcccctttattggtcatctatcaggacaccaggcggccctcaggcatccctgcgcgattctcaggctccaactcggggaacacagccactctgaccatcagcgggacccagactagagatgaatctacctattactgtatgacgtgggacgtcgacactacctcgatgattttcggcggagggaccaagctgaccgtccta

SEQ ID NO: 44 - protein, 114 aa, Artificial Sequence
  Wild-Type Mature FAM19A1 Protein
MLLCHGSLQHTFQQHHLHRPEGGTCEVIAAHRCCNKNRIEERSQTVKCSCLPGKVAGTTRNRPSCVDASIVIGKWWCEMEPCLEGEECKTLPDNSGWMCATGNKIKTTRIHPRT

SEQ ID NO: 45 - protein, 114 aa, Artificial Sequence
  FAM19A1 Mutant M1
MLLCHGSLQHTFQQHHLHRPEGGTCEVIAAHRCCNKNRIEERSQTVKCSCFSGQVAGTTRNRPSCVDASIVIGKWWCEMEPCLEGEECKTLPDNSGWMCATGNKIKTTRIHPRT

SEQ ID NO: 46 - protein, 114 aa, Artificial Sequence
  FAM19A1 Mutant M2
MLLCHGSLQHTFQQHHLHRPEGGTCEVIAAHRCCNKNRIEERSQTVKCSCLPGKVAGTTRAKPSCVDASIVIGKWWCEMEPCLEGEECKTLPDNSGWMCATGNKIKTTRIHPRT

SEQ ID NO: 47 - protein, 114 aa, Artificial Sequence
  FAM19A1 Mutant M3
MLLCHGSLQHTFQQHHLHRPEGGTCEVIAAHRCCNKNRIEERSQTVKCSCLPGKVAGTTRNRPSCVDASIVLQRWWCEMEPCLEGEECKTLPDNSGWMCATGNKIKTTRIHPRT

SEQ ID NO: 48 - protein, 114 aa, Artificial Sequence
  FAM19A1 Mutant M4
MLLCHGSLQHTFQQHHLHRPEGGTCEVIAAHRCCNKNRIEERSQTVKCSCLPGKVAGTTRNRPSCVDASIVIGKWWCQMEPCLPGEECKTLPDNSGWMCATGNKIKTTRIHPRT

SEQ ID NO: 49 - protein, 114 aa, Artificial Sequence
  FAM19A1 Mutant M5
MLLCHGSLQHTFQQHHLHRPEGGTCEVIAAHRCCNKNRIEERSQTVKCSCLPGKVAGTTRNRPSCVDASIVIGKWWCEMEPCLEGEECKVLPDLSGWMCATGNKIKTTRIHPRT

SEQ ID NO: 50 - protein, 114 aa, Artificial Sequence
  FAM19A1 Mutant M6
MLLCHGSLQHTFQQHHLHRPEGGTCEVIAAHRCCNKNRIEERSQTVKCSCLPGKVAGTTRNRPSCVDASIVIGKWWCEMEPCLEGEECKTLPNNSGWSCSSGHKIKTTRIHPRT

SEQ ID NO: 51 - protein, 114 aa, Artificial Sequence
  FAM19A1 Mutant M7
MLLCHGSLQHTFQQHHLHRPEGGTCEVIAAHRCCNKNRIEERSQTVKCSCLPGKVAGTTRNRPSCVDASIVIGKWWCEMEPCLEGEECKTLPDNSGWMCATGNKIKTTKVTRRT

SEQ ID NO: 52 - protein, 19 aa, Artificial Sequence
  Synthetic FAM19A1 peptide
CHGSLQHTFQQHHLHRPEG

(19) United States
(12) Patent Application Publication
(10) Pub. No.: US 2023/0104540 A1
(43) Pub. Date: Apr. 6, 2023

(54) USE OF ANTI-FAM19A1 ANTAGONISTS FOR TREATING CENTRAL NERVOUS SYSTEM DISEASES

(71) Applicant: NEURACLE SCIENCE CO., LTD., Seoul (KR)

(72) Inventors: Bongcheol KIM, Seongnam-Si (KR); Dong Sik KIM, Seoul (KR); Soon-gu KWON, Seoul (KR); Nui HA, Gyeonggi-do (KR); Jae Young SEONG, Seoul (KR); Jong Ik HWANG, Seoul (KR); Hyo Jung YONG, Seoul (KR); Eun Bee CHO, Seoul (KR)

(73) Assignee: NEURACLE SCIENCE CO., LTD., Seoul (KR)

(21) Appl. No.: 17/905,453

(22) PCT Filed: Mar. 2, 2021
(86) PCT No.: PCT/IB2021/051704

§ 371 Date: Sep. 1, 2022

Related U.S. Application Data
(60) Provisional application No. 62/984,166, filed on Mar. 2, 2020.

(51) Int. Cl. C07K 16/28; A61P 25/04; A61K 9/00

(52) U.S. Cl. CPC C07K 16/28; A61P 25/04; A61K 9/0019; A61K 9/0056

(57) ABSTRACT

The present disclosure relates to a method of treating a disease or disorder associated with an abnormality in CNS function. Also provided is a method for diagnosing and/or identifying a subject having an abnormality in CNS function. FAM19A1 antagonists that can be used with the present disclosures are also provided.

### CROSS-REFERENCE TO RELATED APPLICATIONS

This PCT application claims the priority benefit of U.S. Provisional Application No. 62/984,166, filed Mar. 2, 2020, which is incorporated herein by reference in its entirety.

### STATEMENT OF GOVERNMENT SUPPORT

This work (Grants No. C0558337) was supported by project for Cooperative R&D between Industry, Academy, and Research Institute funded Korea Ministry of SMEs and Startups in 2017.

### REFERENCE TO SEQUENCE LISTING SUBMITTED ELECTRONICALLY

The content of the electronically submitted sequence listing in ASCII text file (Name: 3763.017PC01_SeqListing_ST25.txt; Size: 32,234 bytes; and Date of Creation: Mar. 1, 2021) filed with the application is incorporated herein by reference in its entirety.

### FIELD OF THE DISCLOSURE

The present disclosure provides antagonists (e.g., antibodies) that specifically bind to family with sequence similarity 19, member A1 (FAM19A1), compositions comprising such antagonists, and methods of using such antagonists for preventing and/or treating central nervous system diseases and abnormalities.

### BACKGROUND OF THE DISCLOSURE

Diseases and disorders of the central nervous system (CNS) comprise a heterogeneous group of disorders that are typically of unknown etiology and pathogenesis. At present, there exists no cure for CNS disorders, such as Alzheimers disease (AD), Parkinson's disease (PD), Huntington's Disease (HD), as well as traumatic brain injury (TBI), neuropathic pain, and glaucoma. Indeed, in most instances, available treatments for CNS diseases offer relatively small symptomatic benefit but remain palliative in nature. Moreover, with increasing life expectancy and population growth worldwide, the number of individuals afflicted with CNS diseases and disorders is predicted to further increase. Feigin, V. L., et al., Lancet Neurol 16(11): 877-897 (2017). As such, there remains a need for more effective treatment options for CNS-related diseases and disorders.

### BRIEF SUMMARY OF THE DISCLOSURE

Provided herein is an antagonist that specifically binds to a family with sequence similarity 19, member A1 (FAM19A1) (“FAM19A1 antagonist”), for use in therapy. In some aspects, the FAM19A1 antagonist is capable of treating a disease or disorder in a subject in need thereof.

In some aspects, the disease or disorder comprises a central nervous system (CNS)-related disease or disorder. In some aspects, the CNS-related disease or disorder is associated with an abnormal neural circuit. In some aspects, the CNS-related disease or disorder comprises a mood disorder, psychiatric disorder, or both. In certain aspects, the CNS-related disease or disorder comprises an anxiety, depression, post-traumatic stress disorder (PTSD), bipolar disorder, attention deficit/hyperactivity disorder (ADHD), autism, schizophrenia, neuropathic pain, glaucoma, addiction, arachnoid cyst, catalepsy, encephalitis, epilepsy/seizures, Locked-in syndrome, meningitis, migraine, multiple sclerosis, myelopathy, Alzheimer's disease, Huntington's disease, Parkinson's disease, Amyotrophic lateral sclerosis (ALS), Batten disease, Tourette's syndrome, traumatic brain injury, cerebrospinal damage, stroke, tremors (essential or Parkinsonian), dystonia, intellectual disability, brain tumor, or combinations thereof.

In some aspects, the CNS-related disease or disorder is anxiety, depression, PTSD, or combinations thereof. In some aspects, the FAM19A1 antagonist is capable of improving one or more symptoms associated with anxiety and/or depression (e.g., increasing the locomotor activity of the subject and/or increasing the subject's ability to respond to an external stress).

In some aspects, the CNS-related disease or disorder that can be treated with the present disclosure is a glaucoma. In certain aspects, the FAM19A1 antagonist is capable of reducing, ameliorating, or inhibiting inflammation associated with a glaucoma. In some aspects, the FAM19A1 antagonist is capable of improving a retinal potential in a retina. In some aspects, the glaucoma is selected from the group consisting of an open-angle glaucoma, angle-closure glaucoma, normal-tension glaucoma (“NTG”), congenital glaucoma, secondary glaucoma, pigmentary glaucoma, pseudoexfoliative glaucoma, traumatic glaucoma, neovascular glaucoma, irido comeal endothelial syndrome, uveitic glaucoma, and combinations thereof. In some aspects, the glaucoma is associated with an optic nerve damage, a retinal ganglion cell (“RGC”) loss, a high intraocular pressure (“IOP”), an impaired blood-retina barrier, and/or an increase in a level of microglia activity within a retina and/or optic nerve of the subject. In some aspects, the glaucoma is caused by a mechanical damage to an optic nerve head and/or an increase in a level of inflammation within a retina and/or an optic nerve of the subject.

In some aspects, the FAM19A1 antagonist is capable of delaying an onset of retinal nerve cell degeneration within the subject. In some aspects, the FAM19A1 antagonist is capable of reducing a loss of retinal ganglion cells and/or restoring a retinal ganglion cell number within the retina of the subject. In certain aspects, the loss of retinal ganglion cells is reduced by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist). In some aspects, the retinal ganglion cell number is restored by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist). In some aspects, the FAM19A1 antagonist is capable of protecting nerve connections of an inner plexiform layer of the retina of the subject.

In some aspects, the CNS-related disease or disorder that can be treated with a FAM19A1 antagonist disclosed herein is a neuropathic pain.

In some aspects, the FAM19A1 antagonist is capable of increasing a threshold or latency to an external stimulus in a subject in need thereof. In certain aspects, the external stimulus is a mechanical stimulus. In some aspects, the external stimulus is a thermal stimulus. In some aspects, the threshold or latency to the external stimulus is increased by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist).

In some aspects, the FAM19A1 antagonist is capable of increasing or regulating a sensory nerve conduction velocity in a subject in need thereof. In certain aspects, the sensory nerve conduction velocity is increased by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist).

In some aspects, the neuropathic pain is a central neuropathic pain or a peripheral neuropathic pain. In some aspects, the neuropathic pain is associated with a physical injury, an infection, diabetes, cancer therapy, alcoholism, amputation, weakness of a muscle in the back, leg, hip, or face, trigeminal neuralgia, multiple sclerosis, shingles, spine surgery, or any combination thereof. In some aspects, the neuropathic pain comprises carpal tunnel syndrome, central pain syndrome, degenerative disk disease, diabetic neuropathy, phantom limb pain, postherpetic neuralgia (shingles), pudendal neuralgia, sciatica, low back pain, trigeminal neuralgia, or any combination thereof. In certain aspects, the neuropathic pain is caused by a compression of a nerve. In some aspects, the diabetic neuropathy is diabetic peripheral neuropathy. In some aspects, the neuropathic pain is sciatica.

In some aspects, the FAM19A1 antagonist is capable of regulating or improving a central nervous system function in a subject in need thereof. In some aspects, the central nervous system function comprises a limbic system related function, olfactory system related function, sensory system related function, visual system related function, or combinations thereof.

In some aspects, the FAM19A1 antagonist is capable of reducing an expression level of FAM19A1 protein and/or an expression level of FAM19A1 mRNA in a brain region. In certain aspects, the brain region comprises cerebral cortex, hippocampus, hypothalamus, midbrain, prefrontal cortex, amygdala (e.g., lateral amygdaloid nucleus and basomedial amygdaloid nucleus), piriform cortex, anterior olfactory nucleus, lateral entorhinal cortex, habenula, or combinations thereof.

In some aspects, the FAM19A1 antagonist is capable of reducing an expression level of FAM19A1 protein and/or an expression level of FAM19A1 mRNA in a retina region. In certain aspects, the retina region comprises a ganglion cell layer (GCL) or inner plexiform layer (INL).

In some aspects, the FAM19A1 antagonist is capable of reducing an expression level of FAM19A1 protein and/or an expression level of FAM19A1 mRNA in a spinal cord region. In certain aspects, the spinal cord region comprises dorsal horn.

In some aspects, the expression level of FAM19A1 protein and/or the expression level of FAM19A1 mRNA is reduced by at least about 5%, at least about 10%, at least about 20%, at least about 30%, at least about 40%, at least about 50%, at least about 60%, at least about 70%, at least about 80%, or at least about 90% or more, compared to a reference (e.g., corresponding value in a subject that did not receive the FAM19A1 antagonist or corresponding value in the subject prior to administering the FAM19A1 antagonist).

In some aspects, the FAM19A1 antagonist is capable of regulating, inducing, or increasing a differentiation of neural stem cells in a subject in need thereof.

In some aspects, the FAM19A1 antagonist is capable of increasing a neurite outgrowth in a differentiated neural stem cell compared to a reference (e.g., corresponding value in a subject who did not receive the FAM19A1 antagonist or corresponding value in the subject prior to administering the FAM19A1 antagonist). In some aspects, the neurite outgrowth is increased by at least about 5%, at least about 10%, at least about 20%, at least about 30%, at least about 40%, at least about 50%, at least about 60%, at least about 70%, at least about 80%, or at least about 90% or more compared to the reference.

Also disclosed herein is a method of diagnosing an abnormality in a central nervous system (CNS) function in a subject in need thereof comprising contacting a FAM19A1 antagonist with a sample of the subject and measuring a FAM19A1 protein level or a FAM19A1 mRNA level in the sample. The present application further provides a method of identifying a subject with an abnormality in a central nervous system (CNS) function comprising contacting a FAM19A1 antagonist with a sample of the subject and measuring a FAM19A1 protein level or a FAM19A1 mRNA level in the sample.

In some aspects, the contacting and the measuring is performed in vitro.

In some aspects, the CNS function comprises a limbic system related function, olfactory system related function, sensory system related function, visual system related function, or combinations thereof. In certain aspects, the abnormality in a CNS function is associated with an abnormal neural circuit.

In some aspects, the abnormality in a CNS function is associated with a CNS-related disease or disorder. In some aspects, the CNS-related disease or disorder comprises a mood disorder, psychiatric disorder, or both. In certain aspects, the CNS-related disease or disorder comprises an anxiety, depression, post-traumatic stress disorder (PTSD), bipolar disorder, attention deficit/hyperactivity disorder (ADHD), autism, schizophrenia, neuropathic pain, glaucoma, addiction, arachnoid cyst, catalepsy, encephalitis, epilepsy/seizures, Locked-in syndrome, meningitis, migraine, multiple sclerosis, myelopathy, Alzheimer's disease, Huntington's disease, Parkinson's disease, Amyotrophic lateral sclerosis (ALS), Batten disease, Tourette's syndrome, traumatic brain injury, cerebrospinal damage, stroke, tremors (essential or Parkinsonian), dystonia, intellectual disability, brain tumor, or combinations thereof. In some aspects, the CNS-related disease or disorder is anxiety, depression, PTSD, or combinations thereof. In some aspects, the CNS-related disease or disorder is glaucoma, neuropathic pain, or both.

In some aspects, the abnormality in a central nervous system function is associated with an increase in the FAM19A1 protein level and/or in the FAM19A1 mRNA level in the sample compared to a reference (e.g., corresponding value in a sample of a subject who does not suffer from an abnormality in a central nervous system function, e.g., a healthy subject). In some aspects, the FAM19A1 protein level and/or FAM19A1 mRNA level is increased by at least about 5%, at least about 10%, at least about 20%, at least about 30%, at least about 40%, at least about 50%, at least about 60%, at least about 70%, at least about 80%, or at least about 90% or more, compared to the reference.

In some aspects, the abnormality in a central nervous system function is associated with a decrease in the FAM19A1 protein level and/or in the FAM19A1 mRNA level in the sample compared to a reference (e.g., corresponding value in a sample of a subject who does not suffer from an abnormality in a central nervous system function, e.g., a healthy subject). In some aspects, the FAM19A1 protein level and/or FAM19A1 mRNA level is decreased by at least about 5%, at least about 10%, at least about 20%, at least about 30%, at least about 40%, at least about 50%, at least about 60%, at least about 70%, at least about 80%, or at least about 90% or more, compared to the reference.

In some aspects, the FAM19A1 protein level is measured by an immunohistochemistry, Western blot, radioimmunoassay, enzyme linked immunosorbent assay (ELISA), radioimmunodiffusion, immunoprecipitation assay, Ouchterlony immunodiffusion method, rocket immunoelectrophoresis, tissue immunostaining method, complement fixation assay, FACS, protein chip, or combinations thereof. In some aspects, the FAM19A1 mRNA level is measured by a reverse transcription polymerase chain reaction (RT-PCR), a real time polymerase chain reaction, a Northern blot, or combinations thereof.

In some aspects, the sample comprises a tissue, cell, blood, serum, plasma, saliva, urine, cerebral spinal fluid (CSF), or combinations thereof.

In some aspects, a method of diagnosing or identifying disclosed herein further comprises administering a FAM19A1 antagonist to the subject if the FAM19A1 protein level and/or FAM19A1 mRNA level is increased compared to the reference. In some aspects, a method of diagnosing or identifying disclosed herein further comprises administering an agonist against FAM19A1 (“FAM19A1 agonist”) if the FAM19A1 protein level and/or FAM19A1 mRNA level is decreased compared to the reference.

In some aspects, the FAM19A1 agonist is a FAM19A1 protein. In some aspects, the FAM19A1 antagonist is an antisense oligonucleotide, siRNA, shRNA, miRNA, dsRNA, aptamer, PNA that specifically targets FAM19A1, or a vector including the same. In some aspects, the FAM19A1 antagonist is an anti-FAM19A1 antibody, a polynucleotide encoding the anti-FAM19A1 antibody, a vector comprising the polynucleotide thereof, a cell comprising the polynucleotide thereof, or any combination thereof. In some aspects, the FAM19A1 antagonist is an anti-FAM19A1 antibody.

In some aspects, a subject is a male subject.

Provided herein is an anti-FAM19A1 antibody or antigen-binding fragment thereof (“an anti-FAM19A1 antibody”) that exhibits a property selected from: (a) binds to soluble human FAM19A1 with a K_{D} of 10 nM or less as measured by ELISA; (b) binds to membrane bound human FAM19A1 with a K_{D} of 10 nM or less as measured by ELISA; or (c) both (a) and (b).

In some aspects, the anti-FAM19A1 antibody cross-competes for binding to a human FAM19A1 epitope with a reference antibody comprising a heavy chain CDR1, CDR2, and CDR3 and a light chain CDR1, CDR2, and CDR3,
(i) wherein the heavy chain CDR1 comprises the amino acid sequence set forth in SEQ ID NO: 10, the heavy chain CDR2 comprises the amino acid sequence set forth in SEQ ID NO: 11, the heavy chain CDR3 comprises the amino acid sequence set forth in SEQ ID NO: 12, the light chain CDR1 comprises the amino acid sequence set forth in SEQ ID NO: 13, the light chain CDR2 comprises the amino acid sequence set forth in SEQ ID NO: 14, and the light chain CDR3 comprises the amino acid sequence set forth in SEQ ID NO: 15;
(ii) wherein the heavy chain CDR1 comprises the amino acid sequence set forth in SEQ ID NO: 4, the heavy chain CDR2 comprises the amino acid sequence set forth in SEQ ID NO: 5, the heavy chain CDR3 comprises the amino acid sequence set forth in SEQ ID NO: 6, the light chain CDR1 comprises the amino acid sequence set forth in SEQ ID NO: 7, the light chain CDR2 comprises the amino acid sequence set forth in SEQ ID NO: 8, and the light chain CDR3 comprises the amino acid sequence set forth in SEQ ID NO: 9;
(iii) wherein the heavy chain CDR1 comprises the amino acid sequence set forth in SEQ ID NO: 16, the heavy chain CDR2 comprises the amino acid sequence set forth in SEQ ID NO: 17, the heavy chain CDR3 comprises the amino acid sequence set forth in SEQ ID NO: 18, the light chain CDR1 comprises the amino acid sequence set forth in SEQ ID NO: 19, the light chain CDR2 comprises the amino acid sequence set forth in SEQ ID NO: 20, and the light chain CDR3 comprises the amino acid sequence set forth in SEQ ID NO: 21; or
(iv) wherein the heavy chain CDR1 comprises the amino acid sequence set forth in SEQ ID NO: 22, the heavy chain CDR2 comprises the amino acid sequence set forth in SEQ ID NO: 23, the heavy chain CDR3 comprises the amino acid sequence set forth in SEQ ID NO: 24, the light chain CDR1 comprises the amino acid sequence set forth in SEQ ID NO: 25, the light chain CDR2 comprises the amino acid sequence set forth in SEQ ID NO: 26, and the light chain CDR3 comprises the amino acid sequence set forth in SEQ ID NO: 27.

In some aspects, the anti-FAM19A1 antibody binds to the same FAM19A1 epitope as a reference antibody comprising a heavy chain CDR1, CDR2, and CDR3 and a light chain CDR1, CDR2, and CDR3,
(i) wherein the heavy chain CDR1 comprises the amino acid sequence set forth in SEQ ID NO: 10, the heavy chain CDR2 comprises the amino acid sequence set forth in SEQ ID NO: 11, the heavy chain CDR3 comprises the amino acid sequence set forth in SEQ ID NO: 12, the light chain CDR1 comprises the amino acid sequence set forth in SEQ ID NO: 13, the light chain CDR2 comprises the amino acid sequence set forth in SEQ ID NO: 14, and the light chain CDR3 comprises the amino acid sequence set forth in SEQ ID NO: 15;
(ii) wherein the heavy chain CDR1 comprises the amino acid sequence set forth in SEQ ID NO: 4, the heavy chain CDR2 comprises the amino acid sequence set forth in SEQ ID NO: 5, the heavy chain CDR3 comprises the amino acid sequence set forth in SEQ ID NO: 6, the light chain CDR1 comprises the amino acid sequence set forth in SEQ ID NO: 7, the light chain CDR2 comprises the amino acid sequence set forth in SEQ ID NO: 8, and the light chain CDR3 comprises the amino acid sequence set forth in SEQ ID NO: 9;
(iii) wherein the heavy chain CDR1 comprises the amino acid sequence set forth in SEQ ID NO: 16, the heavy chain CDR2 comprises the amino acid sequence set forth in SEQ ID NO: 17, the heavy chain CDR3 comprises the amino acid sequence set forth in SEQ ID NO: 18, the light chain CDR1 comprises the amino acid sequence set forth in SEQ ID NO: 19, the light chain CDR2 comprises the amino acid sequence set forth in SEQ ID NO: 20, and the light chain CDR3 comprises the amino acid sequence set forth in SEQ ID NO: 21; or
(iv) wherein the heavy chain CDR1 comprises the amino acid sequence set forth in SEQ ID NO: 22, the heavy chain CDR2 comprises the amino acid sequence set forth in SEQ ID NO: 23, the heavy chain CDR3 comprises the amino acid sequence set forth in SEQ ID NO: 24, the light chain CDR1 comprises the amino acid sequence set forth in SEQ ID NO: 25, the light chain CDR2 comprises the amino acid sequence set forth in SEQ ID NO: 26, and the light chain CDR3 comprises the amino acid sequence set forth in SEQ ID NO: 27

In some aspects, the anti-FAM19A1 antibody binds to at least one epitope selected from the group consisting of D112, M117, A119, T120, N122, and combinations thereof.

In some aspects, the anti-FAM19A1 antibody comprises a heavy chain CDR1, CDR2, and CDR3 and a light chain CDR1, CDR2, and CDR3, wherein the heavy chain CDR3 comprises the amino acid sequence set forth in SEQ ID NO: 12, 6, 18, or 24. In certain aspects, the heavy chain CDR1 comprises the amino acid sequence set forth in SEQ ID NO: 10, 4, 16, or 22. In further aspects, the heavy chain CDR2 comprises the amino acid sequence set forth in SEQ ID NO: 11, 5, 17, or 23. In some aspects, the light chain CDR1 comprises the amino acid sequence set forth in SEQ ID NO: 13, 7, 19, or 25. In some aspects, the light chain CDR2 comprises the amino acid sequence set forth in SEQ ID NO: 14, 8, 20, or 26. In certain aspects, the light chain CDR3 comprises the amino acid sequence set forth in SEQ ID NO: 15, 9, 21, or 27.

In some aspects, the anti-FAM19A1 antibody comprises a heavy chain CDR1, CDR2, and CDR3 and a light chain CDR1, CDR2, and CDR3, wherein
(i) the heavy chain CDR1, CDR2, and CDR3 comprises the amino acid sequence set forth in SEQ ID NOs: 10-12, respectively, and the light chain CDR1, CDR2, and CDR3 comprises the amino acid sequence set forth in SEQ ID NOs: 13-15, respectively;
(ii) the heavy chain CDR1, CDR2, and CDR3 comprises the amino acid sequence set forth in SEQ ID NOs: 4-6, respectively, and the light chain CDR1, CDR2, and CDR3 comprises the amino acid sequence set forth in SEQ ID NOs: 7-9, respectively;
(iii) the heavy chain CDR1, CDR2, and CDR3 comprises the amino acid sequence set forth in SEQ ID NOs: 16-18, respectively, and the light chain CDR1, CDR2, and CDR3 comprises the amino acid sequence set forth in SEQ ID NOs: 19-21, respectively; or
(iv) the heavy chain CDR1, CDR2, and CDR3 comprises the amino acid sequence set forth in SEQ ID NOs: 22-24, respectively, and the light chain CDR1, CDR2, and CDR3 comprises the amino acid sequence set forth in SEQ ID NOs: 25-27, respectively.

In some aspects, the anti-FAM19A1 antibody comprises a heavy chain variable domain comprising the amino acid sequence set forth in SEQ ID NO: 30, 28, 32, or 34 and/or a light chain variable domain comprising the amino acid sequence set forth in SEQ ID NO: 31, 29, 33, or 35.

In some aspects, the anti-FAM19A1 antibody comprises a heavy chain variable domain comprising the amino acid sequence set forth in SEQ ID NO: 30 and a light chain variable domain comprising the amino acid sequence set forth in SEQ ID NO: 31. In certain aspects, the anti-FAM19A1 antibody comprises a heavy chain variable domain comprising the amino acid sequence set forth in SEQ ID NO: 28 and a light chain variable domain comprising the amino acid sequence set forth in SEQ ID NO: 29. In some aspects, the anti-FAM19A1 antibody comprises a heavy chain variable domain comprising the amino acid sequence set forth in SEQ ID NO: 32 and a light chain variable domain comprising the amino acid sequence set forth in SEQ ID NO: 33. In some aspects, the anti-FAM19A1 antibody comprises a heavy chain variable domain comprising the amino acid sequence set forth in SEQ ID NO: 34 and a light chain variable domain comprising the amino acid sequence set forth in SEQ ID NO: 35.

In some aspects, the anti-FAM19A1 antibody comprises a heavy chain variable region (VH) and a light chain variable region (VL), wherein the VH comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 30, 28, 32, or 34; and/or wherein the VL comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 31, 29, 33, or 35.

In some aspects, the anti-FAM19A1 antibody is a chimeric antibody, human antibody, or humanized antibody. In some aspects, the anti-FAM19A1 antibody comprises a Fab, Fab′, F(ab′)2, Fv, or single chain Fv (scFv). In some aspects, the anti-FAM19A1 antibody is selected from the group consisting of an IgG1, an IgG2, an IgG3, an IgG4, a variant thereof, and any combination thereof. In some aspects, the anti-FAM19A1 antibody is an IgG1 antibody. In some aspects, the anti-FAM19A1 antibody comprises a constant region without a Fc function.

In some aspects, the anti-FAM19A1 antibody is linked to an agent, thereby forming an immunoconjugate. In certain aspects, the anti-FAM19A1 antibody is formulated with a pharmaceutically acceptable carrier.

In some aspects, the FAM19A1 antagonist useful for the methods disclosed herein is an anti-FAM19A1 antibody provided herein.

In some aspects, the FAM19A1 antagonist is administered intravenously, orally, parenterally, intrathecally, intra-cerebroventricularly, pulmonarily, intramuscularly, subcutaneously, intravitreally, or intraventricularly. In some aspects, a subject is a human.

Also provided herein is a nucleic acid comprising a nucleotide sequence encoding the anti-FAM19A1 antibody of the present disclosure. Present disclosure also provides a vector comprising the nucleic acid disclosed herein and one or more promoters operably linked to the nucleic acid. Present disclosure further provides a cell comprising the nucleic acid or the vector described herein. Disclosed herein is a composition comprising the anti-FAM19A1 antibody of the present disclosure, and a carrier. Present disclosure provides a kit comprising the anti-FAM19A1 antibody of the present disclosure, and instructions for use.

Provided herein is a method of producing an anti-FAM19A1 antibody comprising culturing the cell disclosed herein under suitable condition and isolating the anti-FAM19A1 antibody.

### BRIEF DESCRIPTION OF THE DRAWINGS

FIG. 1 shows ELISA results for the binding of the positive poly scFv-phage antibody pool from each round of bio-panning as described in Example 2. The positive poly scFv-phage antibody pools shown include those from (i) round 1 of bio-panning (“1′ Fc”), (ii) round 2 of bio-panning (“2′ mFc”), and (iii) round 3 of bio-panning (“3′ Fc”). M13 phage #38 and the whole library were used as controls. For each of the scFv-phage antibody pool shown, binding to FAM19A1-Fc, FAM19A1-mFc, and non-FAM19A1 protein (ITGA6-Fc) are shown, from left to right.

FIG. 2 shows the ELISA results of the binding of individual mono scFv-phage clones isolated from round 3 of bio-panning to FAM19A1 protein. The clones shown include (from left to right): 1A1, 1A2, 1A3, 1A4, 1A5, 1A6, 1A7, 1A8, 1A9, 1A10, 1A11, 1A12, 1B1, 1B2, 1B3, 1B4, 1B5, 1B6, 1B7, 1B8, 1B9, 1B10, 1B11, 1B12, 1C1, 1C2, 1C3, 1C4, 1C5, 1C6, 1C7, 1C8, 1C9, 1C10, 1C11, 1C12, 1D1, 1D2, 1D3, 1D4, 1D5, 1D6, 1D7, 1D8, 1D9, 1D10, 1D11, and 1D12. For each of the antibody clones, the bar on the left represents binding to FAM19A1-Fc. For each of the antibody clones, the bar on the right represents binding to a negative control (non-FAM19A1-Fc).

FIG. 3 shows the BstNI fingerprinting analysis of different mono scFv-phage clones isolated from round 3 of bio-panning. The clones shown include (from left to right): 1A11, 1C1, M, 2A10, 2C9, 2D12, 2E1, 2G7, 2G8, 2H4, 2H9, 2H11, 2H12, 3A4, 3A5, 3A8, 3A11, 3B6, 3B8, 3B10, 3C5, 3D1, 3D11, 3D12, 3E2, 3E7, 3E12, 3F12, 3G3, 3G4, 3G10, 3G12, 3H2, 3H3, 3H9, 4A2, 4D1, 4E10, 4G8, 4H11, 5A3, 5C1, 5C3, 5C6, 5E11, 5G1, 6E12, and 7G8. The following clones were able to specifically bind to FAM19A1 (i.e., did not bind to non-FAM19A1 control proteins) with high affinity: 1A11, 1C1, 2G7, and 3A8. Antibody clones 2C9, 5A3, and 2E1 did not bind specifically to FAM19A1, was not a monophage, or bound FAM19A1 with low affinity, respectively.

FIG. 4 shows the ELISA results for the binding of different mono scFv-phage clones to both FAM19A1 and non-FAM19A1 proteins. For each of the clones, binding to the following proteins are shown (from left to right): (i) FAM19A1-MYC/DKK (Origene), (ii) FAM19A1-N-Fc, (iii) FAM19A1-N-mFc, (iv) ITGA6-Fc, (v) CD58-Fc, (vi) hRAGE-Fc, (vii) AITR-Fc, (viii) c-Fc, and (ix) mFc. The three FAM19A1 proteins differ only in the tags used to detect binding.

FIGS. 5A, 5B, 5C, and 5D show different characteristics of the anti-FAM19A1 IgG1 antibodies produced as described in Example 3. FIG. 5A provides a schematic of the construction of the expression vectors used to produce the anti-FAM19A1 IgG1 antibodies. FIG. 5B provides the purity and mobility of the antibodies as confirmed by SDS-PAGE analysis. FIG. 5C provides the productivity data. FIG. 5D provides the binding analysis of the antibodies to FAM19A1 protein as measured by ELISA. The table below the graph provides the Kd value. In FIGS. 5B, 5C, and 5D, the anti-FAM19A1 IgG1 antibodies shown include clones 1A11, 1C1, 2G7, and 3A8.

FIG. 6 shows the ELISA results for the binding of different anti-FAM19A1 antibody clones to FAM19A1 mutants M1-M7. Wild-type FAM19A1 and PBS were used as controls. For each of the FAM19A1 proteins, the five bars shown correspond to anti-FAM19A1 antibody clones (i) 1A11 (“A1-1A11-Ybio”), (ii) 1C1 (“FAM19A1-1C1”), (iii) F41H5, (iv) D6 (“D6-a-Fam19A1”), and E1 (“E1-a-Fam19A1”) (moving from left to right).

FIG. 7 shows FAM19A1 mRNA expression in different tissues from mice. Tissues shown include those from different brain regions (i.e., cerebral cortex, cerebellum, midbrain, spinal cord, hippocampus, olfactory bulb, hypothalamus, and pituitary) and peripheral tissues (i.e., heart, liver, spleen, stomach, small intestine, testis, kidney and lung). The brain region tissues are boxed.

FIGS. 8A, 8B, 8C, 8D, 8E, 8F, 8G, and 8H show FAM19A1 expression in the FAM19A1 LacZ Knock-In (KI) mice that was developed, as described in the Examples. FIG. 8A provides a schematic diagram of the FAM19A1 LacZ KI mouse gene construction. LacZ gene sequence was inserted right behind the start codon in the exon 2 of FAM19A1 gene. This gene construct was expressed using native FAM19A1 promotor and the resulting product is β-galactosidase without any part of FAM19A1 protein due to poly A tail after LacZ sequence. Therefore, homozygous FAM19A1 LacZ KI mouse was considered as a complete knock-out of FAM19A1. E1, exon 1; E2, exon 2; E3, exon 3; E4, exon 4; E5, exon 5; lacZ, lacZ gene; neo, amino 3′-glycosyl phosphotransferase gene; pA, poly-A tail. FIG. 8B provides genomic DNA PCR results comparing β-galactosidase (343 bp) and FAM19A1 (243 bp) in wild-type, FAM19A1 LacZ KI (+/−), and FAM19A1 LacZ KI (−/−) animals. FIG. 8C provides RT-PCR results comparing FAM19A1 expression in both the cortex (CTX) and hippocampus (HIP) of the different animals. FIG. 8D provides a comparison of endogenous FAM19A1 protein expression in the cortex (CTX) and hippocampus (HIP) of the different animals using FAM19A1 specific antibody. Exposure time during development with ECL solution; 30 min for FAM19A1 and 1 min for β-actin. FIGS. 8E (cortex) and 8F (hippocampus) provide a quantitative analysis of the results shown in FIG. 8D. FIG. 8G shows both FAM19A1 mRNA and protein expression in various regions of brain from wild-type animals. Exposure time during development with ECL solution; 30 min for FAM19A1 and 1 min for β-actin. Regions shown include: (i) cortex (CTX), (ii) hippocampus (HIP), (iii) olfactory bulb (OB), (iv) cerebellum (CB), (v) thalamus+hypothalamus (TH+HYP), (vi) midbrain (MB), and (vii) pons (PO). FIG. 8H provides a quantitative analysis of the results shown in FIG. 8G. In FIGS. 8E, 8F, and 8H, “a.u.” refers to arbitrary unit. Data are presented as means f standard errors of the means (SEM). **p<0.01, ***p<0.001 versus WT by one-way analysis of variance (ANOVA) with Bonferroni post hoc tests.

FIGS. 9A, 9B, and 9C show whole brain X-gal staining in FAM19A1 LacZ KI (−/−) mice during different developmental stages. FIG. 9A provides a comparison of beta-galactosidase expression in WT and FAM19A1 LacZ KI mice at day 12.5 of embryonic development (E12.5). FIG. 9B shows beta-galactosidase expression at days 14.5, 16.5, and 18.5 of embryonic development in the FAM19A1 LacZ KI mouse. FIG. 9C shows beta-galactosidase expression at days 0.5, 2.5, 7.5, 14.5, and 56.6 post birth in the FAM19A1 LacZ KI mouse. In FIGS. 9A, 9B, and 9C, the scale bar=2 mm.

FIGS. 10A and 10B provide X-gal staining of embryonic and postnatal FAM19A1 LacZ knock-in (KI) (+/−) mouse brains. FIG. 10A shows detection of X-gal signals in the coronal brain sections at days 14.5 (E14.5) and 18.5 (E18.5) of embryonic development. FIG. 10B shows detection of X-gal signals in various regions at days 0.5 (P0.5), 7.5 (P7.5), and 14.5 (P14.5) post-birth. ACo, anterior cortical amygdaloid nucleus; Amy, amygdala; AO, anterior olfactory nucleus; Au, auditory cortex; BMA; basomedial amygdaloid nucleus, anterior part; CEn, entorhinal cortex; CPf, piriform cortex; FR, fasciculus retroflexus; Hip, hippocampus; LS, lateral septal nucleus; M, motor cortex; MGV, medial geniculate nucleus, ventral part; Op, optic nerve layer of the superior colliculus; PF, pontine flexure; PMCo, posteromedial cortical amygdaloid nucleus; Pn, pontine nuclei; PrL, prelimbic cortex; RMC, red nucleus, magnocellular part; S, somatosensory cortex; V, visual cortex.

FIGS. 11A, 11B, and 11C show FAM19A1 expression pattern in the brain of different adult mice. In FIG. 11A, X-gal precipitates (red) were detected in the subset of cortical L2-3 CUX1-positive neurons (green) in FAM19A1 LacZ KI mice. In FIG. 11B, β-galactosidase (green) were identified in the subset of cortical L5 CTIP2-positive neurons (magenta) in FAM19A1 LacZ KI mice. Arrowheads indicate X-gal or β-galactosidase expressing cortical marker cells. FIG. 11C show X-gal staining in coronal brain sections of adult mice as measured by immunohistochemistry. The different panels represent a different brain region from the relevant animals. AO, anterior olfactory nucleus; Apir, amygdalopiriform transition area BLA, basomedial amygdaloid nucleus; BLP, basolateral amygdaloid nucleus, posterior part; CEn, entorhinal cortex; CPf, piriform cortex; D3V, dorsal 3rd ventricle; FrA, frontal association cortex; L2-3, cortical layer 2-3; L5, cortical layer 5; CA1, 2 and 3, field of CA1, CA2 and CA3 regions of the hippocampus; LaDL, lateral amygdaloid nucleus; LHb, lateral habenular nucleus; LO, lateral orbital cortex; LS, lateral septal nucleus; LV, lateral ventricle; MGN, medial geniculate nucleus; MO, medial orbital cortex; Op, optic nerve layer of the superior colliculus; PMCo, posteromedial cortical amygdaloid nucleus; PrL, prelimbic cortex; Py, pyramidal cell layer of the hippocampus; RG, retrosplenial granular cortex; VO, ventral orbital cortex.

FIG. 12 provides FAM19A1 expression (as shown by X-gal staining) in the adult mouse brain, spinal cord, and dorsal root ganglia. Panels A-G show X-gal stained coronal sections of different brain regions of heterozygous FAM19A1 LacZ knock-in (KI) mouse. Panels H and I show X-gal stained coronal sections of different brains of homozygous FAM19A1 LacZ KI mouse. Panels J-M show X-gal stained coronal spinal cord sections of the heterozygous FAM19A1 LacZ KI mouse. Panel N shows X-gal stained dorsal root ganglia of the heterozygous FAM19A1 LacZ KI mouse. 3V, 3rd ventricle; 7N, facial nucleus; cp, cerebral peduncle; DC, dorsal cochlear nucleus; Ecu, external cuneate nucleus; ic, internal capsule; IPDL, interpeduncular nucleus, dorsolateral subnucleus; lfp, longitudinal fasciculus of the pons; LPO, lateral preoptic area; LRt, lateral reticular nucleus; ml, medial lemniscus; MPOM, medial preoptic nucleus, medial part; MVeMC, medial vestibular nucleus, magnocellular part; MVePC, medial vestibular nucleus, parvicellular part; Pn, pontine nuclei; Po, posterior thalamic nuclear group; Pr, prepositus nucleus; py, pyramidal tract; RtTg, reticulotegmental nucleus of the pons; Sp5I, spinal trigeminal nucleus, interpolar part; Sp50, spinal trigeminal nucleus, oral part; SpVe, spinal vestibular nucleus; SuVe, superior vestibular nucleus; VMH, ventromedial hypothalamic nucleus; X, nucleus X.

FIG. 13 shows FAM19A1 mRNA expression in the developing and mature wild-type (WT) rat brains by in situ hybridization using FAM19A1 mRNA probes. The age of the rat for each of the brain sections shown are provided on the bottom right corner of each panel: (i) day 14.5 of embryonic development (E14.5), (ii) day 16.5 of embryonic development (E16.5), (iii) day 18.5 of embryonic development (E18.5), (iv) day 0.5 post-birth (P0.5), (v) day 7.5 post-birth (P7.5), (vi) day 14.5 post-birth (P14.5), (vii) day 21.5 post birth (P21.5), and (viii) different views of the adult brain (sagittal, horizontal, and coronal). Amy, amygdala; AO, anterior olfactory nucleus; Cer, cerebellum; CTX, cortex; Hb, habenular; Hip, hippocampus; Mes, mesencephalon; SC, spinal cord; Tel, telencephalon; Th, thalamus.

FIG. 14 provides a table showing the number and percentages of offspring generated from the heterozygous FAM19A1 LacZ KI parents.

FIGS. 15A, 15B, 15C, 15D, 15E, 15F, 15G, 15H, 15I, and 15J provide comparison of morphological differences between wild-type and FAM19A1 LacZ knock-in (KI) mice. FIGS. 15A and 15B show changes in body weight with age in male and female mice, respectively. FIG. 15C provides a whole-mount view of WT and FAM19A1 (−/−) adult mouse brain. FIG. 15D shows motor cortical layers in Nissl-stained brain tissues of WT, heterozygous FAM19A1 LacZ KI (FAM19A1+/−), and homozygous FAM19A1 LacZ KI (FAM19A1−/−) mice. FIGS. 15E, 15F, and 15G provide total brain length, cerebral cortical length, and brain width, respectively, of WT (n=9), FAM19A1+/−(n=8) and FAM19A1−/− (n=8) adult mouse brains. FIGS. 15H, 15I, and 15J provide cortical thickness of the motor, somatosensory, and visual cortex, respectively, of WT (n=5), FAM19A1+/−(n=5) and FAM19A1−/− (n=4) adult mice. Data are presented as means f standard errors of the means (SEM). *p<0.05, **p<0.01, ***p<0.001 versus WT or FAM19A1+/− by one-way or two-way analysis of variance (ANOVA) with Bonferroni post hoc tests.

FIGS. 16A and 16B provide comparison of estimated cortical volume (FIG. 16A) and total neuronal cell counts (FIG. 16B) in the cerebral cortex of wild-type (WT), FAM19A1+/−, and FAM19A1−/− of adult mouse brain. Data are presented as means f standard errors of means (SEM).

FIGS. 17A, 17B, 17C, 17D, 17E, and 17F provide comparison of cortical layer thickness in wild-type, FAM19A1+/−, and FAM19A1−/− adult mice. FIGS. 17A, 17B, and 17C show the cortical layer thickness of motor, somatosensory, and visual cortex, respectively. FIGS. 17D, 17E, and 17F show the thickness ratio of each cortical layer versus the total cortical thickness in the motor, somatosensory, and visual cortex, respectively. The different cortical layers are provided on the x-axis. For each of the cortical layers (x-axis), the bars (going from left to right) represent wild-type (WT), FAM19A1+/−, and FAM19A1−/− adult mice, respectively. Data are presented as means f standard errors of means (SEM). *p<0.05, **p<0.01, ***p<0.001 versus WT mice by one-way analysis of variance (ANOVA).

FIGS. 18A, 18B, 18C, and 18D provide comparison of neuronal cell density in the motor cortical layers of wild-type, FAM19A1+/−, and FAM19A1−/− adult mice. In FIG. 18A, NeuN was used as a marker for neuronal cells. The different cortical layers are identified as L1, L2-3, L4, L5, and L6. FIGS. 18B, 18C, and 18D provide quantitative comparison of the neuronal cell density, volume, and total NeuN-positive cells, respectively, in each of the cortical layers shown in FIG. 18A. In FIGS. 18B, 18C, and 18D, for each of the cortical layers (x-axis), the bars (going from left to right) represent wild-type (WT), FAM19A1+/−, and FAM19A1−/− adult mice, respectively. Data are presented as means f standard errors of means (SEM).

FIGS. 19A, 19B, 19C, 19D, and 19E provide comparison of the number of cortical layer glial cells in the motor cortex of wild-type, FAM19A1+/−, and FAM19A1−/− adult mice. In FIG. 19A, GFAP (green) positive astrocytes and Iba1 (red) positive microglia were detected in the motor cortex. In FIG. 19B, Olig2 positive oligodendrocytes (exemplary positive cells indicated by white arrows) were identified in the motor cortex. FIGS. 19C, 19D, and 19E show the number of GFAP-positive cells, Iba1-positive cells, and Olig2-positive cells, respectively, in the motor cortical layers. In FIGS. 19C, 19D, and 19E, for each of the cortical layers (x-axis), the bars (going from left to right) represent wild-type (WIT), FAM19A1+/−, and FAM19A1−/− adult mice, respectively. Data are presented as means f standard errors of means (SEM).

FIGS. 20A, 20B, 20C, 20D, 20E, 20F, 20G, and 20H provide comparison of hyperactivity in wild-type, FAM19A1+/−, and FAM19A1−/− adult mice. FIGS. 20A and 20B show the total time in the open arm and total distance traveled, respectively, as measured in the elevated plus maze (EPM) test. FIGS. 20C and 20D show the total time in the center and total distance traveled, respectively, as measured in the open field test (OFT). FIG. 20E provide a simple line tracing of animal movements in the OFT arena. FIG. 20F shows the percentage of immobile time as measured in the tail suspension test (TST). FIGS. 20G and 20H shows spontaneous alteration and total distance traveled, respectively, as measured in the Y-maze test. Data are presented as means f standard errors of the means (SEM). *p<0.05, **p<0.01, ***p<0.001 vs WT or FAM19A1+/− by one-way analysis of variance (ANOVA) with Bonferroni post hoc tests.

FIGS. 21A, 21B, 21C, 21D, 21E, and 21F provide comparison of short-term and long-term memory formation in wild-type, FAM19A1+/−, and FAM19A1−/− adult mice. FIGS. 21A, 21B, and 21C show the total time spent on exploration, object preference, and discrimination index, respectively, as measured in the short-term memory novel object recognition (NOR) test. FIGS. 21D, 21E, and 21F show the total time spent on exploration, object preference, and discrimination index, respectively, as measured in the long-term memory NOR test. In FIGS. 21A, 21B, 21D, and 21E, for each of the animal groups, the bar on the left represent results for the familiar object and the bar on the right represent results for the novel object. Data are presented as means f standard errors of the means (SEM). *p<0.05, **p<0.01, ***p<0.001 versus WT or FAM19A1+/− by one-way analysis of variance (ANOVA) with Bonferroni post hoc tests.

FIGS. 22A, 22B, 22C, and 22D provide comparison of fear response in wild-type, FAM19A1+/−, and FAM19A1−/− adult mice. FIG. 22A shows fear conditioning during the acquisition phase of the Pavlovian fear conditioning test. The arrows show the relevant groups. FIGS. 22B and 22C show the results of contextual and auditory memory tests, respectively, which were conducted 24 hours after the acquisition phase. FIG. 22D shows the results of the innate fear test using synthetic fox feces odor, 2,5-dihydro-2,4,5-trimethylthiazoline (TMT). The arrows show the relevant groups. Data are presented as means f standard errors of the means (SEM). *p<0.05, **p<0.01, ***p<0.001 versus WT by two-way analysis of variance (ANOVA) with Bonferroni post hoc test or Student's t test.

FIGS. 23A and 23B provide a comparison of FAM19A1 and FAM19A5 expression (as evidenced by beta-galactosidase expression) in the brain using FAM19A1 LacZ KI and FAM19A5 LacZ KI mice. FIG. 23A shows a schematic diagram of FAM19A5 LacZ KI mouse gene construction. LacZ gene sequence is inserted by homologous recombination. Resulting product is a fusion-form of FAM19A5 with β-galactosidase. FIG. 23B shows FAM19A1 (left side) and FAM19A5 (right side) expression in the brain. Regions shown include: L2-3 (cortical layer 2 and 3); L5b (cortical layer 5b); CA1 (field of CA1 of hippocampus); CA2 (field of CA2 of hippocampus); CA3 (field of CA3 of hippocampus); DG (dentate gyrus); CC (corpus callosom); CTX (cortex); TH (thalamus); fi (fimbria of the hippocampus). Scale bar=500 μm.

FIG. 24 provides a comparison of neurite outgrowth of differentiated neurons in mouse adult neural stem cells treated with the following: (i) control IgG antibody (left panel); (ii) anti-FAM19A1 antibody (middle panel); or (iii) FAM19A1 protein (right panel).

FIG. 25 shows a comparison of the intraocular pressure in glaucoma-induced animals treated with human IgG1 (“hIgG”; open square) or an anti-FAM19A1 antibody (“FAM19A1 Ab”; closed square). Normal healthy animals (i.e., no glaucoma induction) (“naïve”) were used as control. The intraocular pressure was measured at days 0, 14, and 28 post glaucoma induction. Data are expressed as mean±S.D. “***” indicates a statistically significant difference (p<0.001) compared to the naïve control.

FIG. 26 shows a comparison of the oscillatory potential grade in glaucoma-induced animals treated with human IgG1 (“hIgG”) or an anti-FAM19A1 antibody (“FAM19A1 Ab”). Normal healthy animals (i.e., no glaucoma induction) (“naïve”) were used as control. Data are expressed as mean f S.D. “***” indicates a statistically significant difference (p<0.001) compared to the naïve control. “###” indicates a statistically significant difference (p<0.001) compared to the hIgG group.

FIGS. 27A and 27B shows a comparison of the number of retinal ganglion cells (“RGC”) observed in the retinal ganglion cell layer of glaucoma-induced animals treated with human IgG1 (“hIgG”) or an anti-FAM19A1 antibody (“FAM19A1 Ab”). Normal healthy animals (i.e., no glaucoma induction) (“naïve”) were used as control. FIG. 27A shows the RGC cell count as an absolute number. Data are expressed as mean±S.D. “***” indicates a statistically significant difference (p<0.001) compared to the naïve control. “##” indicates a statistically significant difference (p<0.01) compared to the hIgG group. FIG. 27B shows the fluorescent imaging (at 100× magnification) of the retinal ganglion cell layer from a representative animal in each of the groups.

FIG. 28 shows a comparison of paw withdrawal threshold in chronic constrictive injury (CCI) induced rats treated with saline (open square) or an anti-FAM19A1 antibody (closed square). Normal healthy animals (i.e., no CCI induction) (“naïve”) were used as control. Paw withdrawal threshold was measured at days 7, 14, and 21 post CCI induction. Data are expressed as mean±S.D. “#” indicates a statistically significant difference (p<0.05) compared to the saline group.

FIG. 29 shows a comparison of rotarod latency (time it took the animals to fall off the Rotarod-treadmill as described in the Examples) in chronic constrictive injury (CCI) induced rats treated with saline (open square) or an anti-FAM19A1 antibody (closed square). Normal healthy animals (i.e., no CCI induction) (“naïve”) were used as control. Latency was measured at days 7, 14, and 21 post CCI induction. Data are expressed as mean±S.D. “#” indicates a statistically significant difference (p<0.05) compared to the saline group.

FIGS. 30A, 30B, 30C, 30D, 30E, and 30F show the effect of anti-FAM19A1 treatment on neurite outgrowth and dendritic branching in primary mouse hippocampal neurons. FIGS. 30A and 30B show neurite outgrowth via immunohistochemistry in mouse hippocampal neurons treated with vehicle-control or anti-FAM19A1 antibody, respectively. FIG. 30C shows the average total neurite length (μm). FIG. 30D shows the number of primary neurites. FIG. 30E shows the number of branching points. FIG. 30F provides the number of secondary neurites. In FIGS. 30C-30F, data are expressed as mean±S.D. “*” indicates a statistically significant difference (p<0.05). In FIGS. 30A and 30B, scale bar=20 μm.

FIG. 31 shows the analgesic effect of anti-FAM19A1 monoclonal antibody (A1-1C1) in CCI-induced mechanical allodynia. The analgesic effect is shown as the number of paw withdrawal response out of 10 times the filament was applied to each hind paw at a 10 second interval between each application (“percent withdrawal response frequency”). CCI-induced animals treated with human IgG control antibody (closed circle) and normal healthy animals (i.e., no CCI induction) (naïve, open circle) were used as controls. Paw withdrawal response was measured at days 6, 10, 13, 17, and 20 post CCI induction. Data are expressed as mean±S.D. “*” indicates a statistically significant difference (p<0.01). The arrows indicate when the anti-FAM19A1 antibody was administered (i.e., days 7 and 14 post CCI induction).

FIG. 32 shows the analgesic effect of anti-FAM19A1 monoclonal antibody (A1-1C1) in CCI-induced thermal hyperalgesia. The analgesic effect is shown as withdrawal latency (how long it took the animals to withdrawal their paws in response to the thermal stimulus). CCI-induced animals treated with human IgG control antibody (closed circle) and normal healthy animals (i.e., no CCI induction) (naïve, open circle) were used as controls. Paw withdrawal response was measured at days 6, 10, 13, 17, and 20 post CCI induction. Data are expressed as mean±S.D. The arrows indicate when the anti-FAM19A1 antibody was administered (i.e., days 7 and 14 post CCI induction).

### DETAILED DESCRIPTION OF THE DISCLOSURE

Disclosed herein are antagonists (e.g., monoclonal antibody) that specifically binds to human family with sequence similarity 19, member A1 (FAM19A1) and exhibit one or more of the properties disclosed herein.

To facilitate an understanding of the disclosure disclosed herein, a number of terms and phrases are defined. Additional definitions are set forth throughout the detailed description.

### I. Definitions

Throughout this disclosure, the term “a” or “an” entity refers to one or more of that entity; for example, “an antibody,” is understood to represent one or more antibodies. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

The term “family with sequence similarity 19, member A1” or “FAM19A1” refers to a protein that belongs to the TAFA family (also known as FAM19 family) of five highly homologous proteins. These proteins contain conserved cysteine residues at fixed positions, and are distantly related to MIP-1alpha, a member of the CC-chemokine family. FAM19A1 is predominantly expressed within the central nervous system (brain and spinal cord). See Examples. FAM19A1 is also known as TAFA1 or Chemokine-like protein TAFA-1.

In humans, the gene encoding FAM19A1 is located on chromosome 3. There are two potential isoforms of human FAM19A1 (UniProt: Q7Z5A9): isoform 1 (UniProt: Q7Z5A9-1), which consists of 133 amino acids; and isoform 2 (UniProt: A0A087X2J7), which consists of 52 amino acids and is predicted based on EST data. The amino acid sequences of the two known human FAM19A1 isoforms are provided in Table 1 below.

TABLE 1
Amino Acid Sequences of FAM19A1 Isoforms

| | |
|---|---|
| FAM19A1 Isoform 1 | MAMVSAMSWVLYLWISACAMLLCHGSLQHT |
| (UniProt: Q7Z5A9-1, | FQQHHLHRPEGGTCEVIAAHRCCNKNRIEE |
| canonical sequence) | RSQTVKCSCLPGKVAGTTRNRPSCVDASIV |
| (signal peptide is | IGKWWCEMEPCLEGEECKTLPDNSGWMCAT |
| underlined) | GNKIKTTRIHPRT (SEQ ID NO: 1) |
| FAM19A1 Isoform 2 | PEGGTCEVIAAHRCCNKNRIEERSQTVKCS |
| (UniProt: | CLPGKVAGTTRNRPSCVDGAII |
| A0A087X2J7) | (SEQ ID NO: 2) |

The term “FAM19A1” includes any variants or isoforms of FAM19A1 which are naturally expressed by cells. Accordingly, antagonists (e.g., antibodies) described herein can cross-react with different isoforms in the same species (e.g., different isoforms of human FAM19A1), or cross-react with FAM19A1 from species other than human (e.g., mouse FAM19A1). Alternatively, the antibodies can be specific for human FAM19A1 and cannot exhibit any cross-reactivity with other species. FAM19A1, or any variants and isoforms thereof, can either be isolated from cells or tissues which naturally express them or be recombinantly produced. The polynucleotide encoding human FAM19A1 has the GenBank Accession No. NM_213609.3 and the following sequence:

TABLE 2
Nucleotide Sequence of FAM19A1 (Isoform 1)

| | |
|---|---|
| FAM19A1 Isoform 1 | GATACTTTAGAGCGGAGGGATGTATTGAAACAGACTACTGCTACTTACAGCAC |
| (UniProt: Q7Z5A9-1, | CGTATAGCAGCCCTGCTCCTACATTTTGCTGCCTTACTCTGCCCCGAATGCAC |
| canonical sequence) | TGGAGTGGGGATGGTCCATCGGCAACTATAAACTGATTCTCATCAGGAAACTG |
| | CACATTATCTCCCCATCACTTCAAAGGTCTCGTCAGGCAGAGGTGACGCCAGG |
| | AGATGATTTAAAGGTGAAAATGACAAGGTTTCCACCCCTCAAACCTTGGCTCC |
| | TTTTCTGACAATACAGTCTGAATGAACCCGATGTCTTTTTTTTTACTGTGGAA |
| | ATAGGATCGGAAGAGAGTAACATTTTTTTTTTTTTAATCCTGATAAAGAAGAT |
| | TGTTGGGAAGCTCTTTGAAAAAAAATTTTAAATTGTGGCACAGATGGATTTTA |
| | AAAAGTGTTAGATCTTTCCAATGAACACTAATAGAGTACTCTGCTCTTGGCTG |
| | GATTTTTCAGAGAATGGCAATGGTCTCTGCGATGTCCTGGGTCCTGTATTTGT |
| | GGATAAGTGCTTGTGCAATGCTACTCTGCCATGGATCCCTTCAGCACACTTTC |
| | CAGCAGCATCACCTGCACAGACCAGAAGGAGGGACGTGTGAAGTGATAGCAGC |
| | ACACCGATGTTGTAACAAGAATCGCATTGAGGAGCGGTCACAAACAGTAAAGT |
| | GTTCCTGTCTACCTGGAAAAGTGGCTGGAACAACAAGAAACCGGCCTTCTTGC |
| | GTCGATGCCTCCATAGTGATTGGGAAATGGTGGTGTGAGATGGAGCCTTGCCT |
| | AGAAGGAGAAGAATGTAAGACACTCCCTGACAATTCTGGATGGATGTGCGCAA |
| | CAGGCAACAAAATTAAGACCACGAGAATTCACCCAAGAACCTAACAGAAGCAT |
| | TTGTGGTAGTAAAGGAAAACCAACCCTCTGGAAAATACATTTTGAGAATCTCA |
| | AACATCTCACATATATACAAGCCAAATGGATTTCTTACTTGCACTTTGACTGG |
| | CTACCAGATAATCACAGTGCGTTTACTGTGTGTAACGAAATATCCTACAGTGA |
| | GAAGACACAGCGTTTTGGCAACACCATGGAAAGTGGGCTTAAAAAAGGGTTTT |
| | CTCAGTGAAATTTTTGGGCATCATGAAGAACGATCAACTATCTTCTAATTTGA |
| | ATCTATAGTTACTTTGTACCATTTGAAATATATGTATATATATATATATAATA |
| | TTTTGAAATATTATCTATTCTCTTCAAGAAATGAACAGTACCACAGTTTGAGA |
| | CGGCTGGTGTACCCCTTTGAGTTTTGGATGTTTTGTCTGTTTTGCTTTGTTTT |
| | GTTAGTCATTTCTTTTTCTAACGGCAAGGAAGATATGTGCCCTTTTGAGAATT |
| | CAAGATGGCACTGACACGGGAAGGCCAGCTACAGGTGGACTCCTGGAATTTGA |
| | GGCATCATAATGATACTGAATCAAGAACTTCCTTCTGCTTCTACCAGATGGCC |
| | CAAGGAAGCACATCGTCCTGTTTTATTGCTTTCTACCCTGTGCAATATTAGCA |
| | TGCAAGCTTGGCTTACATAGTCATACTTTATATTCAATTGATATATAATAACC |
| | GTTCTAACCTCTTCCAGGAAAATATTTTTAGAACTACTAGCTTTTCCACTTAG |
| | AAGAAAATGAGGATTCTTAAGGGAGCCACTCCACCATGCTATTAAGACTCTGG |
| | CAGAGTTATGGGTAGGATATGGATCCCTACATGAATAAGTCCTGTAAATACAA |
| | TGTCTTAAGGCTTTGTATAGCTGTCCTAGACTGCAGAAATGTCCTCTGATTAA |
| | ATCCAAAGTCTGGCATCGTTAACTACATAGTGCTGTAGCAACAAGTCTTATCA |
| | TGGCATCTCTTTCTATGTTTGGTTTGCTTTTTCCAAGAGTATTCAGGTCTCCT |
| | CTTGTGAGATAGGAAGGCCATGAAAACAATTAGATTTCAAGATGATCTATGTG |
| | ACCAAATGTTGGACAGCCCTATTAAAGTGGTAAACAACTTCTTTCTAAAAAAA |
| | AAAAAAAAAAAAAAAAAA (SEQ ID NO: 3) |

The term “antagonist against a FAM19A1 protein” or “FAM19A1 antagonist” refers to all antagonists that suppress the expression of the FAM19A1 protein. Such antagonist can be a peptide, nucleic acid, or a compound. In some aspects, a FAM19A1 antagonist comprises an antisense-oligonucleotide, siRNA, shRNA, miRNA, dsRNA, aptamer, PNA (peptide nucleic acid) targeting FAM19A1, or a vector including the same. In some aspects, a FAM19A1 antagonist comprises an antibody, or antigen-binding fragment thereof, that specifically binds to the FAM19A1 protein.

The term “agonist against a FAM19A1 protein” or “FAM19A1 agonist” refers to all agonists that promote the expression of the FAM19A1 protein and/or share the same biological functions as FAM19A1 protein, such that the FAM19A1 activity is increased. In some aspects, a FAM19A1 agonist is a FAM19A1 protein.

The terms “antibody” and “antibodies” are terms of art and can be used interchangeably herein and refer to a molecule with an antigen binding site that specifically binds an antigen. The terms as used herein include whole antibodies and any antigen binding fragments (i.e., “antigen-binding fragments”) or single chains thereof. An “antibody” refers, in some aspects, to a glycoprotein comprising at least two heavy (H) chains and two light (L) chains inter-connected by disulfide bonds, or an antigen-binding fragment thereof. In some aspects, an “antibody” refers to a single chain antibody comprising a single variable domain, e.g., VHH domain. Each heavy chain is comprised of a heavy chain variable region (abbreviated herein as VH) and a heavy chain constant region. In certain naturally-occurring antibodies, the heavy chain constant region is comprised of three domains, CH1, CH2 and CH3. In certain naturally-occurring antibodies, each light chain is comprised of a light chain variable region (abbreviated herein as VL) and a light chain constant region. The light chain constant region is comprised of one domain, CL.

The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system.

The term “Kabat numbering” and like terms are recognized in the art and refer to a system of numbering amino acid residues in the heavy and light chain variable regions of an antibody, or antigen-binding fragment thereof. In certain aspects, the CDRs of an antibody can be determined according to the Kabat numbering system (see. e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects, the CDRs of the antibodies described herein have been determined according to the Kabat numbering scheme.

The phrases “amino acid position numbering as in Kabat,” “Kabat position,” and grammatical variants thereof refer to the numbering system used for heavy chain variable domains or light chain variable domains of the compilation of antibodies in Kabat et al., Sequences of Proteins of Immunological Interest, 5th Ed. Public Health Service, National Institutes of Health, Bethesda, Md. (1991). Using this numbering system, the actual linear amino acid sequence can contain fewer or additional amino acids corresponding to a shortening of, or insertion into, a FW or CDR of the variable domain. For example, a heavy chain variable domain can include a single amino acid insert (residue 52a according to Kabat) after residue 52 of H2 and inserted residues (e.g., residues 82a, 82b, and 82c, etc. according to Kabat) after heavy chain FW residue 82. See TABLE 3.

TABLE 3

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

The Kabat numbering of residues can be determined for a given antibody by alignment at regions of homology of the sequence of the antibody with a “standard” Kabat numbered sequence. Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

IMGT (ImMunoGeneTics) also provides a numbering system for the immunoglobulin variable regions, including the CDRs. See. e.g., Lefranc, M. P. et al., Dev. Comp. Immunol. 27: 55-77(2003), which is herein incorporated by reference. The IMGT numbering system was based on an alignment of more than 5,000 sequences, structural data, and characterization of hypervariable loops and allows for easy comparison of the variable and CDR regions for all species. According to the IMGT numbering schema VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97.

For all heavy chain constant region amino acid positions discussed in the present disclosure, numbering is according to the EU index first described in Edelman et al., 1969, Proc. Natl. Acad. Sci. USA 63(1):78-85, describing the amino acid sequence of myeloma protein EU, which is the first human IgG1 sequenced. The EU index of Edelman et al. is also set forth in Kabat et al., 1991, Sequences of Proteins of Immunological Interest, 5th Ed., United States Public Health Service, National Institutes of Health, Bethesda. Thus, the phrases “EU index as set forth in Kabat” or “EU index of Kabat” and “position . . . according to the EU index as set forth in Kabat,” and grammatical variants thereof refer to the residue numbering system based on the human IgG1 EU antibody of Edelman et al. as set forth in Kabat 1991.

The numbering system used for the variable domains (both heavy chain and light chain) and light chain constant region amino acid sequence is that set forth in Kabat 1991.

Antibodies can be of any type (e.g., IgG, IgE, IgM, IgD, IgA, or IgY), any class (e.g., IgD, IgG2, IgG3, IgG4, IgA1, or IgA2), or any subclass (e.g., IgG1, IgG2, IgG3, and IgG4 in humans; and IgG1, IgG2a, IgG2b, and IgG3 in mice) of immunoglobulin molecule. Immunoglobulins, e.g., IgG1, exist in several allotypes, which differ from each other in at most a few amino acids. An antibody disclosed herein can be from any of the commonly known isotypes, classes, subclasses, or allotypes. In certain aspects, the antibodies described herein are of the IgG1, IgG2, IgG3, or IgG4 subclass or any hybrid thereof. In certain aspects, the antibodies are of the human IgG1 subclass or the human IgG2 or human IgG4 subclass.

“Antibody” includes, by way of example, both naturally-occurring and non-naturally-occurring antibodies; monoclonal and polyclonal antibodies; chimeric and humanized antibodies; human and non-human antibodies; wholly synthetic antibodies; single chain antibodies; monospecific antibodies; multispecific antibodies (including bispecific antibodies); tetrameric antibodies comprising two heavy chain and two light chain molecules; an antibody light chain monomer; an antibody heavy chain monomer; an antibody light chain dimer, an antibody heavy chain dimer; an antibody light chain-antibody heavy chain pair; intrabodies; heteroconjugate antibodies; monovalent antibodies; camelized antibodies; affybodies; anti-idiotypic (anti-Id) antibodies (including, e.g., anti-anti-Id antibodies), and single-domain antibodies (sdAbs), which include binding molecules consisting of a single monomeric variable antibody domain that are fully capable of antigen binding (e.g., a VH domain or a VL domain). Harmen M. M. and Hard H. J. Appl Microbiol Biotechnol. 77(1): 13-22 (2007)).

The terms “antigen-binding portion” and “antigen-binding fragment” of an antibody are used interchangeably and refer to one or more fragments of an antibody that retain the ability to specifically bind to an antigen (e.g., human FAM19A1). Such “fragments” are, for example between about 8 and about 1500 amino acids in length, suitably between about 8 and about 745 amino acids in length, suitably about 8 to about 300, for example about 8 to about 200 amino acids, or about 10 to about 50 or 100 amino acids in length. It has been shown that the antigen-binding function of an antibody can be performed by fragments of a full-length antibody. Examples of binding fragments encompassed within the term “antigen-binding portion” of an antibody, e.g., an anti-FAM19A1 antibody described herein, include (i) a Fab fragment, a monovalent fragment consisting of the VL, VH, CL, and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, and disulfide-linked Fvs (sdFv); (v) a dAb fragment (Ward et al., Nature 341:544-546 (1989)), which consists of a VH domain; and (vi) an isolated complementarity determining region (CDR) or (vii) a combination of two or more isolated CDRs which can optionally be joined by a synthetic linker. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see. e.g., Bird et al., Science 242:423-426 (1988); and Huston et al., Proc. Natl. Acad. Sci. USA 85:5879-5883 (1988). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

As used herein, the terms “variable region” and “variable domain” are used interchangeably and are common in the art. The variable region typically refers to a portion of an antibody, generally, a portion of a light or heavy chain, typically about the amino-terminal 110 to 120 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR).

Without wishing to be bound by any particular mechanism or theory, it is believed that the CDRs of the light and heavy chains are primarily responsible for the interaction and specificity of the antibody with antigen. In certain aspects, the variable region is a human variable region. In certain aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

As used herein, the term “heavy chain” (HC) when used in reference to an antibody can refer to any distinct type, e.g., alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” (LC) when used in reference to an antibody can refer to any distinct type, e.g., kappa (u) or lambda (Q) based on the amino acid sequence of the constant domains. Light chain amino acid sequences are well known in the art. In specific aspects, the light chain is a human light chain.

The terms “VL” and “VL domain” are used interchangeably to refer to the light chain variable region of an antibody.

The terms “VH” and “VH domain” are used interchangeably to refer to the heavy chain variable region of an antibody.

As used herein, the terms “constant region” and “constant domain” are interchangeable and are known in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

An “Fc region” (fragment crystallizable region) or “Fc domain” or “Fc” refers to the C-terminal region of the heavy chain of an antibody that mediates the binding of the immunoglobulin to host tissues or factors, including binding to Fc receptors located on various cells of the immune system (e.g., effector cells) or to the first component (C1q) of the classical complement system. Thus, a Fc region comprises the constant region of an antibody excluding the first constant region immunoglobulin domain (e.g., CH1 or CL). In IgG, IgA and IgD antibody isotypes, the Fc region comprises two identical protein fragments, derived from the second (CH2) and third (CH3) constant domains of the antibody's two heavy chains; IgM and IgE Fc regions comprise three heavy chain constant domains (CH domains 2-4) in each polypeptide chain. For IgG, the Fc region comprises immunoglobulin domains Cγ2 and Cγ3 and the hinge between Cγ1 and Cγ2. Although the boundaries of the Fc region of an immunoglobulin heavy chain might vary, the human IgG heavy chain Fc region is usually defined to stretch from an amino acid residue at position C226 or P230 (or amino acid between these two amino acids) to the carboxy-terminus of the heavy chain, wherein the numbering is according to the EU index as in Kabat. The CH2 domain of a human IgG Fc region extends from about amino acid 231 to about amino acid 340, whereas the CH3 domain is positioned on C-terminal side of a Cm domain in a Fc region, i.e., it extends from about amino acid 341 to about amino acid 447 of an IgG. As used herein, the Fc region can be a native sequence Fc, including any allotypic variant, or a variant Fc (e.g., a non-naturally-occurring Fc). Fc can also refer to this region in isolation or in the context of a Fc-comprising protein polypeptide such as a “binding protein comprising a Fc region,” also referred to as an “Fc fusion protein” (e.g., an antibody or immunoadhesion).

A “native sequence Fc region” or “native sequence Fc” comprises an amino acid sequence that is identical to the amino acid sequence of a Fc region found in nature. Native sequence human Fc regions include a native sequence human IgG1 Fc region; native sequence human IgG2 Fc region; native sequence human IgG3 Fc region; and native sequence human IgG4 Fc region as well as naturally-occurring variants thereof. Native sequence Fc includes the various allotypes of Fes (see. e.g., Jefferis et al., mAbs 1:1 (2009); Vidarsson G. et al. Front Immunol. 5:520 (2014).

An “Fc receptor” or “FcR” is a receptor that binds to the Fc region of an immunoglobulin. FcRs that bind to an IgG antibody comprise receptors of the FcγR family, including allelic variants and alternatively spliced forms of these receptors. The FcγR family consists of three activating (FcγRI, FcγRIII, and FcγRIV in mice; FcγRIA, FcγRIIA, and FcγRIIIA in humans) and one inhibitory (FcγRIIB) receptor. Human IgG1 binds to most human Fc receptors and elicits the strongest Fc effector functions. It is considered equivalent to murine IgG2a with respect to the types of activating Fc receptors that it binds to. Conversely, human IgG4 elicits the least Fc effector functions. Vidarsson G. et al. Front Immunol. 5:520 (published online Oct. 20, 2014).

The constant region can be manipulated, e.g., by recombinant technology, to eliminate one or more effector functions. An “effector function” refers to the interaction of an antibody Fc region with a Fc receptor or ligand, or a biochemical event that results therefrom. Exemplary “effector functions” include C1q binding, complement dependent cytotoxicity (CDC), Fc receptor binding, FcγR-mediated effector functions such as ADCC and antibody dependent cell-mediated phagocytosis (ADCP), and down regulation of a cell surface receptor (e.g., the B cell receptor; BCR). Such effector functions generally require the Fc region to be combined with a binding domain (e.g., an antibody variable domain). Accordingly, the term “a constant region without the Fc function” include constant regions with reduced or without one or more effector functions mediated by Fc region.

Effector functions of an antibody can be reduced or avoided by different approaches. Effector functions of an antibody can be reduced or avoided by using antibody fragments lacking the Fc region (e.g., such as a Fab, F(ab′)₂, single chain Fv (scFv), or a sdAb consisting of a monomeric VH or VL domain). Alternatively, the so-called aglycosylated antibodies can be generated by removing sugars that are linked to particular residues in the Fc region to reduce the effector functions of an antibody while retaining other valuable attributes of the Fc region (e.g., prolonged half-life and heterodimerization). Aglycosylated antibodies can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells). See. e.g., U.S. Pub. No. 20120100140. Another approach is to employ Fc regions from an IgG subclass that have reduced effector function, for example, IgG2 and IgG4 antibodies are characterized by having lower levels of Fc effector functions than IgG1 and IgG3. The residues most proximal to the hinge region in the CH2 domain of the Fc part are responsible for effector functions of antibodies as it contains a largely overlapping binding site for C1q (complement) and IgG-Fc receptors (FcγR) on effector cells of the innate immune system. Vidarsson G. et al. Front Immunol. 5:520 (2014). Accordingly, antibodies with reduced or without Fc effector functions can be prepared by generating, e.g., a chimeric Fc region which comprises a CH2 domain from an IgG antibody of the IgG4 isotype and a CH3 domain from an IgG antibody of the IgG1 isotype, or a chimeric Fc region which comprises hinge region from IgG2 and CH2 region from IgG4 (see. e.g., Lau C. et al. J. Immunol. 191:4769-4777 (2013)), or a Fc region with mutations that result in altered Fc effector functions, e.g., reduced or no Fc functions. Such Fc regions with mutations are known in the art. See, e.g., U.S. Pub. No. 20120100140 and U.S. and PCT applications cited therein; and An et al., mAbs 1:6, 572-579 (2009).

A “hinge”, “hinge domain” or “hinge region” or “antibody hinge region” refers to the domain of a heavy chain constant region that joins the CH1 domain to the CH2 domain and includes the upper, middle, and lower portions of the hinge (Roux et al., J. Immunol. 161:4083 (1998)). The hinge provides varying levels of flexibility between the binding and effector regions of an antibody and also provides sites for intermolecular disulfide bonding between the two heavy chain constant regions. As used herein, a hinge starts at Glu216 and ends at Gly237 for all IgG isotypes (Roux et al., J Immunol 161:4083 (1988)). The sequences of wild-type IgG1, IgG2, IgG3 and IgG4 hinges are known in the art. See, e.g., Kabat E A et al., (1991) Sequences of Proteins of Immunological Interest, Fifth Edition, U.S. Department of Health and Human Services, NIH Publication No. 91-3242; Vidarsson G. et al. Front Immunol. 5:520 (published online Oct. 20, 2014).

The term “CH1 domain” refers to the heavy chain constant region linking the variable domain to the hinge in a heavy chain constant domain. As used herein, a CH1 domain starts at A118 and ends at V215. The term “CH1 domain” includes wildtype CH1 domains, as well as naturally existing variants thereof (e.g., allotypes). CH1 domain sequences of IgG1, IgG2, IgG3, and IgG4 (including wildtype and allotypes) are known in the art. See, e.g., Kabat E A et al., (1991) supra and Vidarsson G. et al. Front Immunol. 5:520 (published online Oct. 20, 2014). Exemplary CH1 domains include CH1 domains with mutations that modify a biological activity of an antibody, e.g., half-life, e.g., described in U.S. Pub. No. 20120100140 and U.S. patents and publications and PCT publications cited therein.

The term “CH2 domain” refers to the heavy chain constant region linking the hinge to the CH3 domain in a heavy chain constant domain. As used herein, a CH2 domain starts at P238 and ends at K340. The term “CH2 domain” includes wildtype CH2 domains, as well as naturally existing variants thereof (e.g., allotypes). CH2 domain sequences of IgG1, IgG2, IgG3, and IgG4 (including wildtype and allotypes) are known in the art. See. e.g., Kabat E A et al., (1991) supra and Vidarsson G. et al. Front Immunol. 5:520 (published online Oct. 20, 2014). Exemplary CH2 domains include CH2 domains with mutations that modify a biological activity of an antibody, e.g., half-life and/or reduced Fc effector function, e.g., described in U.S. Pub. No. 20120100140 and U.S. patents and publications and PCT publications cited therein.

The term “CH3 domain” refers to the heavy chain constant region that is C-terminal to the CH2 domain in a heavy chain constant domain. As used herein, a CH3 domain starts at G341 and ends at K447. The term “CH3 domain” includes wildtype CH3 domains, as well as naturally existing variants thereof (e.g., allotypes). CH3 domain sequences of IgG1, IgG2, IgG3, and IgG4 (including wildtype and allotypes) are known in the art. See. e.g., Kabat E A et al., (1991) supra and Vidarsson G. et al. Front Immunol. 5:520 (published online Oct. 20, 2014). Exemplary CH3 domains include CH3 domains with mutations that modify a biological activity of an antibody, e.g., half-life, e.g., described in U.S. Pub. No. 20120100140 and U.S. patents and publications and PCT publications cited therein.

As used herein, “isotype” refers to the antibody class (e.g., IgG1, IgG2, IgG3, IgG4, IgM, IgA1, IgA2, IgD, and IgE antibody) that is encoded by the heavy chain constant region genes.

“Allotype” refers to naturally-occurring variants within a specific isotype group, which variants differ in a few amino acids (see, e.g., Jefferis et al. (2009) mAbs 1:1). Antibodies described herein can be of any allotype. Allotypes of IgG1, IgG2, IgG3, and IgG4 are known in the art. See, e.g., Kabat E A et al., (1991) supra; Vidarsson G. et al. Front Immunol. 5:520 (published online Oct. 20, 2014); and Lefranc M P, mAbs 1:4, 1-7(2009).

The phrases “an antibody recognizing an antigen” and “an antibody specific for an antigen” are used interchangeably herein with the term “an antibody which binds specifically to an antigen.”

An “isolated antibody,” as used herein, is intended to refer to an antibody which is substantially free of other antibodies having different antigenic specificities (e.g., an isolated antibody that specifically binds to FAM19A1 is substantially free of antibodies that specifically bind antigens other than FAM19A1). An isolated antibody that specifically binds to an epitope of FAM19A1 can, however, have cross-reactivity to other FAM19A1 proteins from different species.

“Binding affinity” generally refers to the strength of the sum total of interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off}can be determined by techniques known to one of ordinary skill in the art, such as immunoassays (e.g., enzyme-linked immunosorbent assay (ELISA)), BIACORE™ or kinetic exclusion assay (KINEXA®).

As used herein, the terms “specifically binds,” “specifically recognizes,” “specific binding,” “selective binding,” and “selectively binds,” are analogous terms in the context of antibodies and refer to molecules (e.g., antibodies) that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIACORE™, KINEXA® 3000 instrument (Sapidyne Instruments, Boise, Id.), or other assays known in the art. In aspect some aspects, molecules that specifically bind to an antigen bind to the antigen with a K_{A} that is at least 2 logs, 2.5 logs, 3 logs, 4 logs or greater than the K_{A} when the molecules bind to another antigen.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 10⁻⁹ M or less, and most preferably between 10⁻⁸ M and 10⁻¹⁰ M or less, when determined by, e.g., immunoassays (e.g., ELISA) or surface plasmon resonance (SPR) technology in a BIACORE™ 2000 instrument using the predetermined antigen, but does not bind with high affinity to unrelated antigens.

As used herein, the term “antigen” refers to any natural or synthetic immunogenic substance, such as a protein, peptide, or hapten. An antigen can be FAM19A1 or a fragment thereof.

An “epitope” is a term in the art and refers to a localized region of an antigen to which an antibody can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). Epitopes formed from contiguous amino acids are typically, but not always, retained on exposure to denaturing solvents, whereas epitopes formed by tertiary folding are typically lost on treatment with denaturing solvents. An epitope typically includes at least 1, 2, 3, 4, 5, 6, 7, 8, 9, 10, 11, 12, 13, 14, 15, or 20 amino acids in a unique spatial conformation. Methods for determining what epitopes are bound by a given antibody (i.e., epitope mapping) are known in the art and include, for example, immunoblotting and immunoprecipitation assays, wherein overlapping or contiguous peptides from (e.g., from FMAM19A5) are tested for reactivity with a given antibody (e.g., anti-FAM19A1 antibody). Methods of determining spatial conformation of epitopes include techniques in the art and those described herein, for example, x-ray crystallography, 2-dimensional nuclear magnetic resonance and HDX-MS (see, e.g., Epitope Mapping Protocols in Methods in Molecular Biology, Vol. 66, G. E. Morris, Ed. (1996)).

In certain aspects, the epitope to which an antibody binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al., Acta Crystallogr D Biol Crystallogr 50(Pt 4): 339-350 (1994); McPherson A Eur J Biochem 189: 1-23 (1990); Chayen N E Structure 5: 1269-1274 (1997); McPherson A J Biol Chem 251:6300-6303 (1976)). Antibody: antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G Acta Crystallogr D Biol Crystallogr 49(Pt 1): 37-60 (1993); Bricogne G Meth Enzymol 276A: 361-423 (1997), ed Carter C W; Roversi P et al., Acta Crystallogr D Biol Crystallogr 56(Pt 10): 1316-1323 (2000)). Mutagenesis mapping studies can be accomplished using any method known to one of skill in the art. See, e.g., Champe M et al., J Biol Chem 270 (1995): 1388-1394 and Cunningham B C & Wells J A Science 244: 1081-1085 (1989) for a description of mutagenesis techniques, including alanine scanning mutagenesis techniques.

The term “epitope mapping” refers to the process of identification of the molecular determinants for antibody-antigen recognition.

The term “binds to the same epitope” with reference to two or more antibodies means that the antibodies bind to the same segment of amino acid residues, as determined by a given method. Techniques for determining whether antibodies bind to the “same epitope on FAM19A1” with the antibodies described herein include, for example, epitope mapping methods, such as, x-ray analyses of crystals of antigen:antibody complexes which provides atomic resolution of the epitope and hydrogen/deuterium exchange mass spectrometry (HDX-MS). Other methods monitor the binding of the antibody to antigen fragments or mutated variations of the antigen where loss of binding due to a modification of an amino acid residue within the antigen sequence is often considered an indication of an epitope component. In addition, computational combinatorial methods for epitope mapping can also be used. These methods rely on the ability of the antibody of interest to affinity isolate specific short peptides from combinatorial phage display peptide libraries. Antibodies having the same VH and VL or the same CDR1, 2 and 3 sequences are expected to bind to the same epitope.

Antibodies that “compete with another antibody for binding to a target” refer to antibodies that inhibit (partially or completely) the binding of the other antibody to the target. Whether two antibodies compete with each other for binding to a target, i.e., whether and to what extent one antibody inhibits the binding of the other antibody to a target, can be determined using known competition experiments. In certain aspects, an antibody competes with, and inhibits binding of another antibody to a target by at least 10%, 20%, 30%, 40%, 50%, 60%, 70%, 80%, 90% or 100%. The level of inhibition or competition can be different depending on which antibody is the “blocking antibody” (i.e., the cold antibody that is incubated first with the target). Competition assays can be conducted as described, e.g., in Ed Harlow and David Lane, Cold Spring Harb Protoc; 2006; doi: 10.1101/pdb.prot4277 or in Chapter 11 of “Using Antibodies” by Ed Harlow and David Lane, Cold Spring Harbor Laboratory Press, Cold Spring Harbor, N.Y., USA 1999. Competing antibodies bind to the same epitope, an overlapping epitope or to adjacent epitopes (e.g., as evidenced by steric hindrance).

Other competitive binding assays include: solid phase direct or indirect radioimmunoassay (RIA), solid phase direct or indirect enzyme immunoassay (EIA), sandwich competition assay (see Stahli et al., Methods in Enzymology 9:242 (1983)); solid phase direct biotin-avidin EIA (see Kirkland et al., J. Immunol. 137:3614 (1986)); solid phase direct labeled assay, solid phase direct labeled sandwich assay (see Harlow and Lane, Antibodies: A Laboratory Manual, Cold Spring Harbor Press (1988)); solid phase direct label RIA using 1-125 label (see Morel et al., Mol. Immunol. 25(1):7 (1988)); solid phase direct biotin-avidin EIA (Cheung et al., Virology 176:546 (1990)); and direct labeled RIA. (Moldenhauer et al., Scand. J. Immunol. 32:77 (1990)).

A “bispecific” or “bifunctional antibody” is an artificial hybrid antibody having two different heavy/light chain pairs and two different binding sites. Bispecific antibodies can be produced by variety of methods including fusion of hybridomas or linking of Fab′ fragments. See. e.g., Songsivilai & Lachmann, Clin. Exp. Immunol. 79:315-321 (1990); Kostelny et al., J. Immunol. 148, 1547-1553 (1992).

The term “monoclonal antibody,” as used herein, refers to an antibody that displays a single binding specificity and affinity for a particular epitope or a composition of antibodies in which all antibodies display a single binding specificity and affinity for a particular epitope. Accordingly, the term “human monoclonal antibody” refers to an antibody or antibody composition that display(s) a single binding specificity and which has variable and optional constant regions derived from human germline immunoglobulin sequences. In some aspects, human monoclonal antibodies are produced by a hybridoma which includes a B cell obtained from a transgenic non-human animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies comprise variable and constant regions that utilize particular human germline immunoglobulin sequences are encoded by the germline genes, but include subsequent rearrangements and mutations which occur, for example, during antibody maturation. As known in the art (see. e.g., Lonberg Nature Biotech. 23(9): 1117-1125 (2005)), the variable region contains the antigen binding domain, which is encoded by various genes that rearrange to form an antibody specific for a foreign antigen. In addition to rearrangement, the variable region can be further modified by multiple single amino acid changes (referred to as somatic mutation or hypermutation) to increase the affinity of the antibody to the foreign antigen. The constant region will change in further response to an antigen (i.e., isotype switch). Therefore, the rearranged and somatically mutated nucleic acid molecules that encode the light chain and heavy chain immunoglobulin polypeptides in response to an antigen cannot have sequence identity with the original nucleic acid molecules, but instead will be substantially identical or similar (i.e., have at least 80% identity).

A “human” antibody (HuMAb) refers to an antibody having variable regions in which both the framework and CDR regions are derived from human germline immunoglobulin sequences. Furthermore, if the antibody contains a constant region, the constant region also is derived from human germline immunoglobulin sequences. The antibodies described herein can include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. The terms “human” antibodies and “fully human” antibodies are used synonymously.

A “humanized” antibody refers to an antibody in which some, most or all of the amino acids outside the CDR domains of a non-human antibody are replaced with corresponding amino acids derived from human immunoglobulins. In some aspects, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

The term “cross-reacts,” as used herein, refers to the ability of an antibody described herein to bind to FAM19A1 from a different species. For example, an antibody described herein that binds human FAM19A15 can also bind another species of FAM19A1 (e.g., mouse FAM19A1). As used herein, cross-reactivity can be measured by detecting a specific reactivity with purified antigen in binding assays (e.g., SPR, ELISA) or binding to, or otherwise functionally interacting with, cells physiologically expressing FAM19A1. Methods for determining cross-reactivity include standard binding assays as described herein, for example, by BIACORE™ surface plasmon resonance (SPR) analysis using a BIACORE™ 2000 SPR instrument (Biacore AB, Uppsala, Sweden), or flow cytometric techniques.

The term “naturally-occurring” as applied to an object herein refers to the fact that an object can be found in nature. For example, a polypeptide or polynucleotide sequence that is present in an organism (including viruses) that can be isolated from a source in nature and which has not been intentionally modified by man in the laboratory is naturally-occurring.

A “polypeptide” refers to a chain comprising at least two consecutively linked amino acid residues, with no upper limit on the length of the chain. One or more amino acid residues in the protein can contain a modification such as, but not limited to, glycosylation, phosphorylation or disulfide bond formation. A “protein” can comprise one or more polypeptides.

“Nucleic acid molecule,” as used herein, is intended to include DNA molecules and RNA molecules. A nucleic acid molecule can be single-stranded or double-stranded, and can be cDNA.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The term “therapeutically effective amount” as used herein refers to an amount of a drug, alone or in combination with another therapeutic agent, effective to “treat” a CNS-related disease or disorder in a subject or reduce the risk, potential, possibility or occurrence of a CNS-related disease or disorder. A “therapeutically effective amount” includes an amount of a drug or a therapeutic agent that provides some improvement or benefit to a subject having or at risk of having a CNS-related disease or disorder. Thus, a “therapeutically effective” amount is an amount that reduces the risk, potential, possibility or occurrence of a CNS-related disease or disorder or provides alleviation, mitigation, and/or reduces at least one indicator, and/or decrease in at least one clinical symptom of a CNS-related disease or disorder. Non-limiting examples of CNS-related disease or disorder are provided elsewhere in the present disclosure.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, the term “central nervous system” (CNS) refers to the part of the nervous system comprising the bran and the spinal cord. Central nervous system can additionally include the retina and the optic nerve (cranial nerve II), as well as the olfactory nerves (cranial nerve I) and olfactory epithelium. The brain is the primary control module of the CNS and can be largely divided into four lobes: (1) temporal lobe (important for processing sensory input and assigning it emotional meaning; establishing long-term memories, and some language perception); (2) occipital lobe (visual processing region of the brain, housing the visual cortex); (3) parietal lobe (integrates sensory information including touch, spatial awareness, and navigation; language perception); and (4) frontal lobe (contains majority of the dopamine-sensitive neurons and therefore, is involved in attention, reward, short-term memory, motivation, and planning).

The brain can be further divided into different regions: (1) basal ganglia (involved in control of voluntary movements, procedural learning, and decisions about which motor activities to carry out; diseases that affect this area include Parkinson's disease and Huntington's disease); (2) cerebellum (involved in precise motor control, language and attention; impairment in this area can cause disrupted motor control, known as ataxia); (3) Broca's area (involved in language processing; damage to this area can cause speech impediments); (4) corpus callosum (broad band of nerve fibers that join the left and right hemispheres; dyslexic children are known to have smaller corpus callosums); (5) medualla oblongata (involved in involuntary functions, such as vomiting, breathing, sneezing, and maintaining correct blood pressure); (6) hypothalamus (secretes various neurohormones and influences body temperature control, thirst, and hunger); (7) thalamus (receives sensory and motor input and relays the information to the rest of the cerebral cortex); and (8) amygdala (located within the temporal lobe and involved in decision-making, memory, and emotional responses).

As used herein, the term “spinal cord” refers to the long, thing, tubular structure made up of nervous tissue, that extends from the medualla oblongata in the brainstem to the lumbar region of the vertebral column. Non-limiting examples of spinal cord functions include: (1) connects a large part of the peripheral nervous system to the brain and is responsible for transmitting signals between the brain and the peripheral tissues; and (2) acts as a minor coordinating center responsible for some simple reflexes like the withdrawal reflex.

As used herein, the term “neurons” includes a neuron and a portion or portions thereof (e.g., the neuron cell body, an axon, or a dendrite). The term “neuron” denotes nervous system cells that include a central cell body or soma and two types of extensions or projections: dendrites, by which, in general, the majority of neuronal signals are conveyed to the cell body, and axons, by which, in general, the majority of neuronal signals are conveyed from the cell body to effector cells, such as target neurons or muscle.

The term “neurite” refers to any projection from the cell body of a neuron (e.g., axon or dendrite).

As used herein, “administering” refers to the physical introduction of a therapeutic agent or a composition comprising a therapeutic agent to a subject, using any of the various methods and delivery systems known to those skilled in the art. The different routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intralymphatic, intralesional, intracapsular, intraorbital, intracardiac, intradermal, transtracheal, intratracheal, pulmonary, subcutaneous, subcuticular, intraarticular, subcapsular, subarachnoid, intraventricle, intravitreal, epidural, and intrastemal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The term “diagnosis” as used herein refers to methods that can be used to determine or predict whether a patient is suffering from a given disease or condition, thereby identifying a subject who is suitable for a treatment. A skilled artisan can make a diagnosis on the basis of one or more diagnostic marker (e.g., FAM19A1), where the presence, absence, amount, or change in amount of the diagnostic marker is indicative of the presence, severity, or absence of the condition. In some aspects, an increase in FAM19A1 expression, in a biological sample from a subject, is indicative of a CNS-related disease or disorder. The term “diagnosis” does not refer to the ability to determine the presence or absence of a particular disease or disorder with 100% accuracy, or even that a given course or outcome is more likely to occur than not. Instead, the skilled artisan will understand that the term “diagnosis” refers to an increased probability that a certain disease or disorder is present in the subject. In some aspects, the term “diagnosis” includes one or more diagnostic methods of identifying a subject who has a CNS-related disease or disorder. Non-limiting examples of CNS-related disease or disorder are provided elsewhere in the present disclosure.

The composition for diagnosing an abnormality of CNS function includes an agent for measuring the protein level of FAM19A1 or the nucleic acid (e.g., mRNA) level encoding FAM19A1 in a sample of a subject in need thereof (e.g., suspected of having an abnormality of CNS function). Such agents include oligonucleotides having a sequence complementary to FAM19A1 mRNA, a primer, or a nucleic acid probe that specifically binds to FAM19A1 mRNA, and antibodies, or antigen-binding fragments thereof, that specifically bind to the FAM19A1 protein.

As used herein, the term “subject” includes any human or non-human animal. The term “non-human animal” includes all vertebrates, e.g., mammals and non-mammals, such as non-human primates, sheep, dog, cow, chickens, amphibians, reptiles, etc. As described herein (e.g., Examples), the beneficial effects of FAM19A1 antagonists of the present disclosure are not dependent on gender. Accordingly, in some aspects, a subject that can benefit (e.g., improvement in a CNS function or treatment of a CNS-related disease or disorder) from an FAM19A1 antagonist disclosed herein is a male subject. In some aspects, the term “male” refers to an individual with X and Y chromosomes. In some aspects, a subject that can benefit (e.g., improvement in a CNS function or treatment of a CNS-related disease or disorder) from an FAM19A1 antagonist disclosed herein is a female subject. In some aspects, the term “female” refers to an individual with two X chromosomes. In some aspects, a subject that can benefit (e.g., improvement in a CNS function or treatment of a CNS-related disease or disorder) from an FAM19A1 antagonist disclosed herein comprises both male and female subjects.

As used herein, the term “neuron” includes electrically excitable cells that process and transmit information through electrical and chemical signals. Neurons are the major components of the brain and spinal cord of the CNS, and of the ganglia of the peripheral nervous system (PNS), and can connect to each other to form neural networks. A typical neuron is composed of a cell body (soma), dendrites, and an axon. The soma (the cell body) of a neuron contains the nucleus. The dendrites of a neuron are cellular extensions with many branches, where the majority of input to the neuron occurs. The axon is a finer, cable-like projection extending from the soma and carries nerve signals away from the soma and certain types of information back to the soma. The term “promote regrowth of neuron” includes stimulating, promoting, increasing, or activating growth of neurons, preferably after injury or damage.

The term “glaucoma” refers to a group of ocular-related diseases and/or disorders characterized by the progressive loss of retinal ganglion cell death and optic nerve atrophy. The glaucoma may be associated with an optic nerve damage, a retinal ganglion cell (“RGC”) loss, a high intraocular pressure (“IOP”), an impaired blood-retina barrier, and/or an increase in a level of microglia activity within a retina and/or optic nerve of the subject. The glaucoma may be asymptomatic or may have any of the following symptoms associated with the eyes: burning or stinging sensation, tearing, dryness, tiredness, blurry/dim vision, tunnel vision, difficulty seeing in daylight, difficulty seeing in dark places, halos around lights and/or blindness. Lee et al., Arch Ophthalmol 116: 861-866 (1998). The term “glaucoma” includes any and all types of glaucoma regardless of the cause and any and all symptoms of glaucoma.

The term glaucoma includes but is not limited to the following types of glaucoma: open-angle glaucoma, angle-closure glaucoma, normal-tension glaucoma (“NTG”), congenital glaucoma, secondary glaucoma, pigmentary glaucoma, pseudoexfoliative glaucoma, traumatic glaucoma, neovascular glaucoma, irido comeal endothelial syndrome, and/or and uveitic glaucoma. Some of the risk factors include: elevated intraocular pressure, genetic predisposition (e.g., family history of glaucoma, certain ethnicity), age, diabetes, high blood pressure, and/or physical injury to the eye.

The term “inflammation” refers to a complex reaction of the innate immune system in vascularized tissues that involves the accumulation and activation of immune cells (e.g., microglia) and plasma proteins at a site of an injury and/or damage. In healthy individuals, the blood-retina barrier provides a tight barrier that prevents the free flow of material from the blood to the retina and vice versa. However, in glaucoma patients, this barrier is damaged. This vascular dysregulation restricts the blood flow to the optic nerve head of the retinal and allows the production of various inflammatory mediators (e.g., TNF-α, IL-6, IL-9, IL-10, and nitric oxide), which can freely move to the optic nerve head.

As used herein, the term “optic nerve” refers to a paired nerve that transmits visual information from the retina to the brain. In human, optic nerve is derived from optic stalks during the seventh week of development and is composed of retinal ganglion cell axons and glial cells. Optic nerve extends from the optic disc to the optic chiasma and continues as the optic tract to the lateral geniculate nucleus, pretectal nuclei, and superior colliculus. Selhorst, J. B., et al., Semin Neurol 29(1):29-35 (2009).

The term “optic nerve damage” refers to an alteration of the normal structure or function of the optic nerve. The alteration of the normal structure or function of the optic nerve can be the result of any disease, disorder, or injury, including glaucoma. An alteration of the normal function of the optic nerve includes any alteration of the ability of the optic nerve to function appropriately, such as transmit visual information from the retina to the brain. An alteration in function can manifest itself, for example, as loss of visual field, impaired central visual acuity, abnormal color vision, and so forth. Examples of alteration of structure include nerve fiber loss in the retina, abnormal cupping of the optic nerve, and/or loss of cells from the retinal ganglion cell layer of the retina. “Optic nerve damage” as used herein can include optic nerve damage to one or both optic nerves of a subject.

“Retinal ganglion cells” (RGCs) refers to a specific type of neuron located near the innermost layer of the retina (i.e., the ganglion cell layer). These cells play a crucial role in conveying visual information gathered from the photoreceptors to the brain. Sanes et al., Annu Rev Neurosci 38:221-46 (2015). RGCs can vary significantly in terms of their size, connections, and responses to visual stimulation, but they all share the defining property of having a long axon that extends into the brain.

The term “intraocular pressure” (IOP) refers to the pressure that is maintained within the eye. The anterior chamber of the eye is bounded by the cornea, iris, pupil and lens. It is filled with aqueous humor, a watery fluid responsible for providing the cornea and lens with oxygen and nutrients. The aqueous humor provides the necessary pressure to help maintain the shape of the eye. When normal secretion of the aqueous humor is interrupted, intraocular pressure is affected.

The term “microglia” or “microglial cells” refers to a type of glial cells that are present within the retina. These cells behave like macrophages and are constantly surveying the surrounding microenvironment for foreign antigen and/or injury. Upon such recognition, the microglia become activated and quickly respond to the foreign antigen and/or injury by phagocytosing any potentially harmful debris to limit the damage, secreting inflammatory mediators, and interacting with other immune cells to generate an effective immune response. Kettenmann et al., Physiol Rev 91(2): 461-553 (2011). Activated microglia are distinguishable from those that are in a resting state by the increased surface expression of Iba-1. Microglial cells are also involved in programmed cell death in the developing retina, and nerve growth factor (NGF) released by microglia may induce retinal neuronal cell death. Ashwell et al., Visual Neuroscience 2(5): 437-448 (1989).

The term “neuropathic pain” refers to a pain due to an injury, damage, and/or improper function affecting any level of the central nervous system (CNS) and/or the peripheral nervous system. The term “neuropathic pain” includes any and all types of neuropathic pain regardless of the cause and any and all symptoms of neuropathic pain.

Neuropathic pain includes central neuropathic pain and peripheral neuropathic pain. As used herein, the term “central neuropathic pain” refers to pain resulting from a disorder, congenital defect, or injury to the central nervous system (i.e., the brain or spinal cord). As used herein, the term “peripheral neuropathic pain” refers to pain resulting from an injury or an infection of the peripheral sensory nerves.

Symptoms of neuropathic pain can include persistent/chronic pain, spontaneous pain, as well as allodynia (e.g., a painful response to a stimulus that normally is not painful), hyperalgesia (e.g., an accentuated response to a painful stimulus that usually causes only a mild discomfort, such as a pin prick), hyperesthesia (e.g., excessive physical sensitivity to stimuli, especially of the skin), or hyperpathia (e.g., where a short discomfort becomes a prolonged severe pain). In some aspects, symptoms can be long-lasting and persist after resolution of the primary cause, if one was present. Merck Manual, Neuropathic Pain, available at merckmanuals.com/professional/neurologic-disorders/pain/neuropathic-pain; Campbell J. N. and Meyer R. A. Neuron 52(1): 77-92 (2006).

As used herein, “mononeuropathy” is a peripheral neuropathy involving loss of movement or sensation to an area caused by damage or destruction to a single peripheral nerve or nerve group. Mononeuropathy is most often caused by an injury or trauma to a local area, which, e.g., results in prolonged pressure/compression on a single nerve. However, certain systemic disorders (e.g., mononeuritis multiplex) can also cause mononeuropathy. In some aspect, the injury or trauma to a local area causes destruction of the myelin sheath (covering) of the nerve or of part of the nerve cell (the axon), which can slow down or prevent the conduction of impulses through the nerve. In some aspect, the mononeuropathy can affect any part of the body. Examples of mononeuropathic pain include, without limitation, a sciatic nerve dysfunction, a common peroneal nerve dysfunction, a radial nerve dysfunction, an ulnar nerve dysfunction, a cranial mononeuropathy VI, a cranial mononeuropathy VII, a cranial mononeuropathy III (compression type), a cranial mononeuropathy III (diabetic type), an axillary nerve dysfunction, a carpal tunnel syndrome, a femoral nerve dysfunction, a tibial nerve dysfunction, a Bell's palsy, a thoracic outlet syndrome, a carpal tunnel syndrome, and a sixth (abducent) nerve palsy. Finnerup N. B. et al., Pain 157(8): 1599-1606 (2016); National Institute of Neurological Disorders and Stroke, Peripheral Neuropathy Fact Sheet, available at ninds.nih.gov/disorders/peripheralneuropathy/detail_jeripheralneuropathy.htm.

As used herein, “polyneuropathy” is a peripheral neuropathy involving the loss of movement or sensation to an area caused by damage or destruction to multiple peripheral nerves. Polyneuropathic pain includes, without limitation, post-polio syndrome, postmastectomy syndrome, diabetic neuropathy, alcohol neuropathy, amyloid, toxins, AIDS, hypothyroidism, uremia, vitamin deficiencies, chemotherapy-induced pain, 2′,3′-didexoycytidine (ddC) treatment, Guillain-Barre syndrome or Fabry's disease. Finnerup N. B. et al., Pain 157(8): 1599-1606 (2016); National Institute of Neurological Disorders and Stroke, Peripheral Neuropathy Fact Sheet, available at ninds.nih.gov/disorders/peripheralneuropathy/detail_jeripheralneuropathy.htm.

The term “a neuropathic pain associated with” a disease or disorder refers to a neuropathic pain that accompanies a disease or disorder (e.g., those disclosed herein), or caused by or resulting from a disease or a disorder (e.g., those disclosed herein).

### II. Methods of the Present Disclosure

### Methods of Treating a Disease or Disorder

Disclosed herein are FAM19A1 antagonists (e.g., antibodies) that can be used in a therapy (e.g., treating a disease or disorder). As described herein, FAM19A1 is expressed in many regions (e.g., neural circuits) of the CNS, and it is thought that abnormal FAM19A1 expression could result in impairment of normal CNS functions. As demonstrated throughout the application (e.g., Examples), applicants have discovered that the FAM19A1 antagonists disclosed herein can be used to improve one or more CNS functions. Applicants have further identified that the beneficial effects of the FAM19A1 antagonists disclosed herein are independent of the subject's gender. Accordingly, in some aspects, a FAM19A1 antagonist disclosed herein can be used to treat a male subject (e.g., suffering from an impaired CNS function). In some aspects, the subject is not a female subject. In some aspects, a FAM19A1 antagonist disclosed herein can be used to treat a female subject (e.g., suffering from an impaired CNS function). In some aspects, a FAM19A1 antagonist disclosed herein can be used to treat both male and female subjects (e.g., suffering from an impaired CNS function).

In some aspects, an FAM19A1 antagonist disclosed herein can be used to treat a CNS-related disease or disorder. In some aspects, the CNS-related disease or disorder that can be treated with the present disclosure is associated with an abnormal neural circuit. As used herein, the term “neural circuit” refers to a population of neurons interconnected by synapses to carry out a specific function when activated. Neural circuits can interconnect to one another to form large scale brain networks. Neural circuits are integral to the proper transmission of information from the brain to the neurons. And, different neural circuits are generally associated with different functions. Abnormalities in one or more neural circuits can result in impaired CNS functions, such as those observed in various types of CNS-related diseases or disorders.

In some aspects, a CNS-related disease or disorder that can be treated with the FAM19A1 antagonists disclosed herein include mood disorders, psychiatric disorders, or both. In some aspects, a CNS-related disease or disorder that can be treated with the present disclosure comprises an anxiety, depression, post-traumatic stress disorder (PTSD), bipolar disorder, attention deficit/hyperactivity disorder (ADHD), autism, schizophrenia, neuropathic pain, glaucoma, addiction, arachnoid cyst, catalepsy, encephalitis, epilepsy/seizures, Locked-in syndrome, meningitis, migraine, multiple sclerosis, myelopathy, Alzheimer's disease, Huntington's disease, Parkinson's disease, Amyotrophic lateral sclerosis (ALS), Batten disease, Tourette's syndrome, traumatic brain injury, cerebrospinal damage, stroke, tremors (essential or Parkinsonian), dystonia, intellectual disability, brain tumor, or combinations thereof.

In some aspects, a CNS-related disease or disorder that can be treated with an FAM19A1 antagonist disclosed herein is a mood disorder. As used herein, the term “mood disorder” refers to any type of mental illnesses that affect an individual's emotional state. As used herein, the term “mood” refers to an internal emotional state of a person. In some aspects, a mood disorder that can be treated with the present disclosure can be characterized by pervasive, prolonged, and disabling exaggerations of mood and affect that are associated with behavioral, physiologic, cognitive, neurochemical and psychomotor dysfunctions. The mood disorder can be associated with a persistent elevated mood (mania), a persistent depressed mood, or a mood which cycles between mania and depression. The mood disorder can be hereditary in nature and/or induced by a secondary factor (e.g., illness, drugs, medication). Examples of mood disorders include, but are not limited to, major depressive disorder (MDD), bipolar disorder (BD), minor depressive disorder, persistent depressive disorder (dysthymia), seasonable affective disorder (SAD), psychotic depression, postpartum depression, brief recurrent depression, premenstrual dysphoria (PMDD), situational depression, atypical depression, anxiety disorder, and cyclothymic disorder.

The term “major depressive disorder” or “MDD,” as used herein, refers to a mood disorder that is characterized by two or more major depressive episodes. Symptoms of MDD can include fatigue, feelings of worthlessness or guilt, impaired concentration or indecisiveness, insomnia or hypersomnia, markedly diminished interest or pleasure in almost all activities, restlessness, recurring thoughts of death or suicide, and significant weight loss or gain (5% weight change). Diagnostic criteria for MDD, along with other mood disorders, can be found, for example, in the Diagnostic and Statistical Manual of Mental Disorders, fourth edition, DSM-VI®-TR, American Psychiatric Association (DSM IV) and are useful for assessing a subject.

The term “bipolar disorder” refers to a mood disorder characterized by alternating periods of extreme moods. A person with bipolar disorder experiences cycling of moods that usually swing from being overly elated or irritable (mania) to sad and hopeless (depression) and then back again, with periods of normal mood in between. Diagnosis of bipolar disorder is described in, e.g., DSM IV. Categories of bipolar disorders include, but are not limited to, bipolar disorder I (mania with or without major depression) and bipolar disorder II (hypomania with major depression). As used herein, the term “mania” or “manic” refers to a disordered mental state of extreme excitement. The term “hypomania” refers to a less extreme manic episode, with lower grade of severity.

As will be apparent from the present disclosure, an FAM19A1 antagonist disclosed herein can be used to treat all types of mood disorders.

In some aspects, an impairment that can be treated with an FAM19A1 antagonist disclosed herein is related to visual system. Accordingly, in some aspects, provided herein is a method of treating a glaucoma in a subject in need thereof, comprising administering a FAM19A1 antagonist to the subject. In some aspects, a FAM19A1 antagonist useful for the present disclosure antisense oligonucleotide, siRNA, shRNA, miRNA, dsRNA, aptamer, PNA that specifically targets FAM19A1, or a vector including the same. In some aspects, a FAM19A1 antagonist is an anti-FAM19A1 antibody, a polynucleotide encoding the anti-FAM19A1 antibody, or a vector comprising the polynucleotide thereof. In some aspects, a FAM19A1 antagonist binds to FAM19A1 protein and reduces FAM19A1 activity. In further aspects, reduced FAM19A1 activity reduces, ameliorates, or inhibits inflammation associated with a glaucoma.

In some aspects, a FAM19A1 antagonist (e.g., anti-FAM19A1 antibody) reduces the loss of retinal ganglion cells (RGCs) and/or restore retinal ganglion cell numbers within the retina of a subject (e.g., glaucoma patient). In certain aspects, loss of retinal ganglion cells is reduced by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist). In some aspects, retinal ganglion cell number is restored by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist).

In some aspects, a FAM19A1 antagonist disclosed herein (e.g., anti-FAM19A1 antibody) delays an onset of retinal nerve cell degeneration with a subject (e.g., glaucoma patient). In some aspects, a FAM19A1 antagonist protects the nerve connections of an inner plexiform layer of a retina of a subject (e.g., glaucoma patient). In some aspects, a FAM19A1 antagonist suppresses inflammation around the optic nerve head of the retina by regulating the activation of microglial cells. In some aspects, a FAM19A1 antagonist disclosed herein (e.g., anti-FAM19A1 antibody) has now effect on reducing elevated intraocular pressure observed in, e.g., a glaucoma subject. However, in some aspects, a FAM19A1 antagonist does increase and/or improve retinal potential in a subject (e.g., glaucoma patient).

In some aspects, glaucoma comprises an open-angle glaucoma, angle-closure glaucoma, normal-tension glaucoma (“NTG”), congenital glaucoma, secondary glaucoma, pigmentary glaucoma, pseudoexfoliative glaucoma, traumatic glaucoma, neovascular glaucoma, irido comeal endothelial syndrome, uveitic glaucoma, or combinations thereof. In certain aspects, glaucoma is associated with an optic nerve damage, retinal ganglion cell (“RGC”) loss, high intraocular pressure (“IOP”), impaired blood-retina barrier, and/or increase in a level of microglia activity within a retina and/or optic nerve of the subject

In some aspects, a method of treating a glaucoma further comprises administering one or more additional agents for treating a glaucoma. In certain aspects, the additional agent is a prostaglandin analog, such as XALATAN®, LUMIGAN®, TRAVATAN Z®. In some aspects, the additional agent is an alpha agonist, such as ALPHAGAN® P and IOPIDINE®. In further aspects, the additional agent is a carbonic anhydrase inhibitors, such as TRUSOPT® and AZOPT®. Such additional agents can be administered prior to, concurrently, or after the FAM19A1 antagonist administration.

In some aspects, an impairment to a CNS function is related to the sensory system, particularly, relating to sensation of touch. Accordingly, in some aspects, the present disclosure provides a method of treating, preventing, or ameliorating a neuropathic pain in a subject in need thereof comprising administering a FAM19A1 antagonist to the subject.

In some aspects, a neuropathic pain is a central neuropathic pain, i.e., a pain due to injury or damage affecting any level of the CNS (e.g., brain injury and spinal cord injury), including the central somatosensory nervous system, or associated with or as a result of a disease or disorder such as stoke, multiple sclerosis, or lateral medullary infarction. In some aspects, a central neuropathic pain can be spontaneous or stimulus-evoked. In some aspects, a central neuropathic pain can involve dynamic mechanical allodynia and cold allodynia. Symptoms of central neuropathic pain include, for example, sensations such as burning, pricking, shooting, squeezing, painful cold, paresthesia and dysesthesia are common (e.g., tingling, pins and needles, cold, and pressing sensations). Distribution of central neuropathic pain includes areas ranging from a small area to large areas, e.g., in the periorbital area, or covering half the body in stroke or the lower body in spinal cord injury, or involving one side of the face and the contralateral side of the body or limbs. Central neuropathic pain due to in spinal cord injury includes “at-level” pain, which is pain perceived in a segmental pattern at the level of injury, and “below-level” pain, which is pain felt below the injury level. In some aspects, the method lessens, reverses, alleviates, ameliorates, inhibits, or slows down or prevents a central neuropathic pain, a symptom associated with the pain, an underlining cause of the pain, or a combination thereof.

In some aspects, a neuropathic pain is a peripheral neuropathic pain, a pain due to injury or damage affecting any level of the peripheral nerves system (e.g., injury of a motor nerve, a sensory nerve, an autonomic nerve, or a combination thereof), or resulting from or associated with a disease or disorder. Injury or damage of a motor nerve is associated with symptoms such as muscle weakness (e.g., weakness of a muscle in the back, leg, hip, or face), painful cramps and fasciculations (uncontrolled muscle twitching visible under the skin), muscle atrophy (severe shrinkage of muscle size), and decreased reflexes. Injury or damage of a sensory nerve damage results in a variety of symptoms including pain and an over sensitization of pain receptors in the skin, leading to allodynia (e.g., severe pain from stimuli that are normally painless).

In some aspects, the method treats one or more types of neuropathic pain comprising administering a FAM19A1 antagonist (e.g., anti-FAM19A1 antibody) to a subject in need thereof. In some aspects, a neuropathic pain that can be treated with a method disclosed herein is a neuralgia, which includes, without limitation, a trigeminal neuralgia (TN) (e.g., a pain within the facial or intraoral trigeminal territory), an atypical trigeminal neuralgia (ATN), an occipital neuralgia, a postherpetic neuralgia (e.g., a pain unilateral distributed in one or more spinal dermatomes or the trigeminal ophthalmic division), a peripheral nerve injury pain (e.g., a pain in the innervation territory of the lesioned nerve, typically distal to a trauma, surgery, or compression), a glossopharyngeal neuralgia (e.g., an irritation of the ninth cranial nerve causing extreme pain in the back of the throat, tongue and ear), a sciatica, a low back pain, and an atypical facial pain. In some aspects, the neuralgia results from or is associated with chemical irritation, inflammation, trauma (including surgery), a compression of a nerve, e.g., by nearby structures (for instance, tumors), or an infection. In some aspects, the neuropathic pain is a deafferentation pain syndrome, which includes, without limitation, an injury to the brain or spinal cord, a post-stroke pain, a phantom pain, a paraplegia, a brachial plexus avulsion injuries, lumbar radiculopathies. In some aspects, a neuropathic pain is a Complex Regional Pain Syndrome (CRPS), including CRPS1 and CRPS 2. A CRPS includes, without limitation, In some aspects, symptoms associated with a CRPS can include severe pain, changes in the nails, bone, and skin; and an increased sensitivity to touch in the affected limb. In some aspects, a neuropathic pain is a neuropathy (e.g., central or peripheral). Non-limiting examples of neuropathy pain include, for example, mononeuropathic pain (mononeuropathy) and polyneuropathic pain (polyneuropathy).

In some aspects, a neuropathic pain results from or is associated with a physical injury, including, for example, (1) a traumatic injury or damage including a nerve compression (e.g., a nerve crush, a nerve stretch, a nerve entrapment or an incomplete nerve transection); (2) a spinal cord injury (e.g., a hemisection of the spinal cord); (3) an injury or damage to a peripheral nerve (e.g., a motor nerve, sensory nerve, or autonomic nerve, or a combination thereof.), (4) a limb amputation; a contusion; an inflammation (e.g., an inflammation of the spinal cord); or a surgical procedure; and (5) repetitive stress, including, for example, repetitive, awkward, and/or forceful activities that require movement of any group of joints for prolonged periods (e.g., ulnar neuropathy and carpal tunnel syndrome). In some aspects, the method treats a neuropathic pain that results from or is associated with an exposure to a toxic agent.

In some aspects, a neuropathic pain results from or is associated one or more diseases or disorders, including, for example, (1) an ischemic event (e.g., a stroke or a heart attack), (2) multiple sclerosis, (3) a metabolic and/or endocrine disease or disorder (e.g., diabetes mellitus, metabolic disease, and acromegaly, a condition caused by overproduction of growth hormone and is characterized by the abnormal enlargement of parts of the skeleton, including the joints, leading to nerve entrapment and pain.), (4) a small vessel disease that causes decreased oxygen supply to the peripheral nerves leading to nerve tissue damage (e.g., vasculitis, namely blood vessel inflammation), (5) an autoimmune disease (e.g., Sjogren's syndrome, lupus, rheumatoid arthritis, and acute inflammatory demyelinating neuropathy, also known as Guillain-Barre syndrome), (6) a kidney disorder, (7) a cancer or tumor (e.g., a neoplastic tumor, neuromas, paraneoplastic syndromes, and toxicity from the chemotherapeutic agents and radiation in cancer treatment), (8) an infection (e.g., an infection by a virus such as herpes varicellazoster (shingles), Epstein-Barr virus, West Nile virus, cytomegalovirus, and herpes simplex virus, AIDS, or an infection by bacteria such as Lyme disease, diphtheria, and leprosy), (9) an inflammatory disorder, (10) a peripheral nerve disorder (e.g., neuroma), (11) a genetic disorder, either hereditary or arise de novo (e.g., Charcot-Marie-Tooth disorders), (12) a mononeuropathy, (13) a polyneuropathy, or a combination thereof. In some aspects, the neuropathic pain results from or is associated with diabetes mellitus (type I or type II). In some aspects, the neuropathic pain is diabetes peripheral neuropathy.

In some aspects, a neuropathic pain results from or is associated with an exposure to an infectious agent including, for example, tick-borne infection, herpes varicellazoster, Epstein-Barr virus, West Nile virus, cytomegalovirus, herpes simplex viruses, AIDS, or to a toxic agent (e.g., a drug, an alcohol, a heavy metal (e.g., lead, arsenic, mercury), or to an industrial agent (e.g., a solvent, fumes from a glue) and nitrous oxide).

In some aspects, a neuropathic pain results from or is associated with a physical injury, an infection, diabetes, cancer therapy, alcoholism, amputation, multiple sclerosis, shingles, spine surgery, sciatica (pain along the sciatic nerve), a low back pain, a neuralgia such as trigeminal neuralgia (e.g., pain within the facial or intraoral trigeminal territory), neuropathy pain such as painful polyneuropathy (e.g., pain in feet, may extend to involve lower legs, thighs, and hands), or a combination thereof. In some aspects, a neuropathic pain is trigeminal neuralgia. In some aspects, a neuropathic pain is associated weakness of a muscle in the back, leg, hip, or face. In some aspects, a neuropathic pain is caused by a compression of a nerve, e.g., a nerve in the leg, foot, hip or a nerve in the facial muscle. In some aspects, a neuropathic pain comprises a sciatic nerve injury. In some aspects, the neuropathic pain is sciatica.

In some aspects, the method of the present disclosure can reverse, alleviate, ameliorate, inhibit, slow down, or prevent one or more symptoms associated with a neuropathic pain. Accordingly, in one aspect, the present disclosure provides a method for improving hyperalgesia in a subject in need thereof, comprising administering to the subject an antagonist against FAM19A1. As used herein, the term “hyperalgesia” refers to an increased or accentuated response to a painful stimulus (e.g., pin prick or hot plate). In some aspects, the hyperalgesia is directed to a mechanical stimuli, such as a pin prick (mechanical hyperalgesia). In other aspects, the hyperalgesia is directed to a thermal stimuli, such as a hot plate (thermal hyperalgesia). In some aspects, the subject in need thereof has a chronic constrictive injury (e.g., sciatica). In some aspects, the subject in need thereof has diabetic peripheral neuropathy.

In some aspects, administering the FAM19A1 antagonist to a subject in need thereof allows the subject to have a higher threshold to a mechanical stimuli compared to a reference control (e.g., neuropathic pain subject who did not receive the FAM19A1 antagonist). As used herein, the term “threshold to a mechanical stimuli” refers to the amount of pressure (from the mechanical stimuli) before a subject responds to the stimuli (e.g., by pulling away). Accordingly, a subject with higher threshold can withstand or resist much greater amount of mechanical stimulation compared to a subject with a lower threshold. In some aspects, the method disclosed herein can increase a subject's threshold to a mechanical stimuli by at least about 5%, at least about 10%, at least about 20%, at least about 30%, at least about 40%, at least about 50%, at least about 60%, at least about 70%, at least about 80%, at least about 90%, at least about 100%, at least about 150%, or at least about 200% compared to reference control (e.g., the subject's threshold prior to administration of the FAM19A1 antagonist).

In some aspects, administering the FAM19A1 antagonist to a subject in need thereof increases the latency (i.e., time interval between the stimulation and the response) of the subject to a thermal stimuli (e.g., hot plate) compared to a reference control (e.g., neuropathic pain subject who did not receive the FAM19A15 antagonist). In some aspects, the method disclosed herein can increase a subject's latency to a thermal stimuli by at least about 5%, at least about 10%, at least about 20%, at least about 30%, at least about 40%, at least about 50%, at least about 60%, at least about 70%, at least about 80%, at least about 90%, at least about 100%, at least about 150%, or at least about 200% compared to reference control (e.g., the subject's threshold prior to administration of the FAM19A1 antagonist).

In other aspect, present disclosure provides a method for improving a sensory nerve conduction velocity in a subject in need thereof. The term “sensory nerve conduction velocity” (SNCV) refers to the rate at which an electrical signal travels through a peripheral nerve. Healthy nerves send electrical signals more quickly and with greater strength than damaged nerves. See Chouhan S., J Clin Diagn Res 10(1):CC01-3 (2016). Therefore, tests that help measure SNCV (e.g., sensory nerve conduction velocity test) can be useful in identifying potential nerve damage and/or dysfunction in a subject. In some aspects, the method disclosed herein can increase a neuropathic pain subject's SNCV by at least about 5%, at least about 10%, at least about 20%, at least about 30%, at least about 40%, at least about 50%, at least about 60%, at least about 70%, at least about 80%, at least about 90%, at least about 100%, at least about 150%, or at least about 200% compared to reference control (e.g., the subject's threshold prior to administration of the FAM19A1 antagonist).

In some aspects, a method of treating a neuropathic pain can further comprise administering an additional agent for treating a neuropathic pain. Non-limiting exemplary agents for treating a neuropathic pain include Venlafaxine (EFFEXOR®), antiepileptic medications such as carbamazepine (CARBATROL®, TEGRETOL®, approved by the FDA for relieving the pain of trigeminal neuralgia), Gabapentin (NEURONTIN®, GRALISE®, approved for the management of postherpetic neuralgia (PHN): pain that lasts one to three months after shingles has healed), and Pregabalin (LYRICA®, approved for PHN, painful diabetic neuropathic pain, and fibromyalgia), sodium channel blocking agent such as lidocaine, adrenergic drugs such as clonidine, phentolamine, phenoxybenzamine, reserpine, dexmedetomidine, opioids such as morphine, and antidepressants such as amitriptyline, imipramine, and duloxetine.

Dose and administration of the one or more additional therapeutic drugs are known in the art, e.g., as instructed by the product label of the respective drug.

In some aspects, the subject being treated is a nonhuman animal, such as a rat or a mouse. In some aspects, the subject being treated is a human.

### Methods of Regulating or Improving a Central Nervous System (CNS) Function

Without being bound by any one theory, in some aspects, a FAM19A1 antagonist disclosed herein can treat a disease or disorder by reducing and/or inhibiting FAM19A1 activity. In some aspects, the reduced and/or inhibited FAM19A1 activity can improve one or more functions of a central nervous system. Accordingly, in some aspects, disclosed herein are methods of regulating or improving one or more functions of a central nervous system in a subject in need thereof, comprising administering a FAM19A1 antagonist to the subject. In some aspects, a FAM19A1 antagonist useful for the present disclosure antisense oligonucleotide, siRNA, shRNA, miRNA, dsRNA, aptamer, PNA that specifically targets FAM19A1, or a vector including the same. In certain aspects, a FAM19A1 antagonist comprises an anti-FAM19A1 antibody, a polynucleotide encoding the anti-FAM19A15 antibody, or a vector comprising the polynucleotide thereof.

As described supra, while there are some overlapping functions, majority of the tissues within the CNS are involved in specific functions, which can be categorized into different groups. Accordingly, in some aspects, central nervous system function comprises a limbic system related function, olfactory system related function, sensory system related function, visual system related function, or combinations thereof.

As used herein, the term “limbic system related function” refers to activities associated with the limbic system. The term “limbic system” refers to the portion of the brain that deals with three key functions: emotions, memories, and arousal (or stimulation). In some aspects, the limbic system comprises the following brain regions: olfactory bulbs, hippocampus, hypothalamus, amygdala, anterior thalamic nuclei, fomix, columns of fomix, mammillary body, septum pellucidum, habenular commissure, cingulate gyrus, parahippocampal gyrus, entorhinal cortex, habenula, and limbic midbrain areas.

As used herein, the term “olfactory system related function,” refers to activities relating to the olfactory system, which refers to the part of the sensory system used for smelling (olfaction).

As used herein, the term “sensory system related function” refers to activities relating to the sensory system. Sensory system comprises sensory neurons (including sensory receptor cells), neural pathways, and parts of the brain involved in sensory perception. In some aspects, functions related to sensory system comprises hearing, touch, taste, balance, or combinations thereof.

As used herein, the term “visual system related function” refers to activities relating to vision. The term “visual system” refers to part of the CNS involved in processing visual detail (e.g., by detecting and interpreting information from visible light), as well as enabling the formation of several non-image photo response functions (e.g., pupillary light reflex (PLR) and circadian photoentrainment). The visual system carries out a number of complex tasks, including the reception of light and the formation of monocular representations; the buildup of a nuclear binocular perception from a pair of two dimensional projections; the identification and categorization of visual objects; assessing distances to and between objects; and guiding body movements in relation to the objects seen.

In connection with the current disclosure, it has been shown that FAM19A1 is expressed in certain brain regions related to CNS functions disclosed herein. See. e.g.. Example 8. Without wishing to be bound by any particular mechanism or theory, it is possible that aberrant expression of FAM19A1 could be responsible for defects in CNS functions.

In some aspects, a FAM19A1 antagonist disclosed herein (e.g., anti-FAM19A1 antibody) can regulate or improve a central nervous system function by reducing FAM19A1 protein expression and/or FAM19A1 mRNA expression in a brain region (e.g., region that is associated with CNS functions disclosed herein). In certain aspects, a brain region comprises cerebral cortex, hippocampus, hypothalamus, midbrain, prefrontal cortex, amygdala (e.g., lateral amygdaloid nucleus and basomedial amygdaloid nucleus), piriform cortex, anterior olfactory nucleus, lateral entorhinal cortex, habenula, or combinations thereof.

In some aspects, a FAM19A1 antagonist (e.g., anti-FAM19A1 antibody) reduces FAM19A1 protein expression and/or FAM19A1 mRNA expression in a retina region. In certain aspects, a retina region comprises a ganglion cell layer (GCL) or inner plexiform layer (INL).

In some aspects, a FAM19A1 antagonist (e.g., anti-FAM19A1 antibody) reduces FAM19A1 protein expression and/or FAM19A1 mRNA expression in a spinal cord region. In certain aspects, a spine region comprises dorsal horn.

In some aspects, after administration of a FAM19A1 antagonist disclosed herein (e.g., anti-FAM19A1 antibody), FAM19A1 protein expression and/or FAM19A1 mRNA expression is reduced by at least about 5%, at least about 10%, at least about 20%, at least about 30%, at least about 40%, at least about 50%, at least about 60%, at least about 70%, at least about 80%, or at least about 90% or more, compared to a reference (e.g., corresponding value in a subject that did not receive the FAM19A1 antagonist or corresponding value in the subject prior to administering the FAM19A1 antagonist). In some aspects, reduced FAM19A1 protein expression and/or FAM19A1 mRNA expression is associated with an improvement in a CNS function.

### Methods of Regulating, Inducing, or Increasing Differentiation of Neurons

Neural stem cells (NSCs) have an ability to divide continuously (i.e., self-renewal ability) and to differentiate into neurons, astrocytes, and oligodendrocytes of the central nervous system. The differentiation process into neurons mainly occurs during the embryonic period, but the differentiation process into glial cells occurs after birth. See Bayer et al., J Comp Neurol 307:499-516 (1991); Miller and Gauthier, Neuron 54:357-369 (2007).

In order to maintain normal CNS functions, a numerical balance of neurons and glial cells (e.g., astrocytes) is essential. While astrocytes do have certain beneficial functions (e.g., provides structural support for neurons, secrete neural growth factors, and help maintain blood-brain barrier), too much astrocyte formation can impede regeneration of neurons and cause inflammatory-mediated damage to the CNS tissues. See Myer et al., Brain 129:2761-2772 (2006); Chen and Swanson, J Cereb Blood Flow Metab 23:137-149 (2003); Cunningham et al., Brain 128: 1931-1942 (2005) and; Faden, Curr Opin Neurol 15:707-712 (2002); see also U.S. Pub. No. 2015/0118230, which is herein incorporated by reference in its entirety.

Gliosis is a phenomenon that commonly occurs in various pathological processes of the central nervous system and is caused by hyperproliferation and activation of astrocytes resulting from neuronal damage. When damage is applied to the central nervous system, normal astrocytes become hypertrophic, reactive astrocytes that increase generation of an intermediate filament protein called glial fibrillary acidic protein (GFAP). Various glial cells including reactive astrocytes undergo hyperproliferation after damage and a solid cell layer named a glial scar that is a product of the healing process is formed. Such gliosis is observed in degenerative brain diseases including Huntington's disease, Parkinson's disease, and Alzheimer's disease, in cerebrospinal damage, and various pathological phenomena of the central nervous system such as strokes and brain tumors. Faideau et al., Hum Mol Genet 19(15):3053-67 (2010); Chen et al., Curr Drug Targets 5:149-157 (2005); Rodriguez et al., Cell Death Differ 16:378-385 (2009); Robel et al., J Neurosci 31(35):12471-12482 (2011); Talbott et al., Exp Neurol 192:11-24 (2005); Shimada et al.. J Neurosci 32(33):7926-40 (2012); Sofroniew and Vinters, Acta Neuropathol 119:7-35 (2010).

Accordingly, without wishing to be bound by any particular mechanism or theory, a CNS function could be improved by promoting and/or regulating the differentiation of neurons, e.g., from neural stem cells. Present disclosure, thus, provides methods of regulating, inducing, or increasing the differentiation and/or maturation of neurons in a subject in need thereof, comprising administering a FAM19A1 antagonist (e.g., anti-FAM19A1 antibody) to the subject. In some aspects, a FAM19A1 antagonist increases a neurite outgrowth in a differentiated neural stem cell (i.e., neurons) compared to a reference (e.g., corresponding value in a subject who did not receive the FAM19A1 antagonist or corresponding value in the subject prior to administering the FAM19A1 antagonist). In certain aspects, neurite outgrowth is increased by at least about 5%, at least about 10%, at least about 20%, at least about 30%, at least about 40%, at least about 50%, at least about 60%, at least about 70%, at least about 80%, or at least about 90% or more compared to the reference.

### Methods of Diagnosing an Abnormality in a Central Nervous System (CNS) Function

Disclosed herein is a method of diagnosing an abnormality in a CNS function in a subject in need thereof, comprising contacting a FAM19A1 antagonist (e.g., anti-FAM19A1 antibody) with a sample of the subject and measuring a FAM19A1 protein level or a FAM19A1 mRNA level in the sample. Also disclosed herein is a method of identifying a subject with an abnormality in a central nervous system function, comprising contacting a FAM19A1 antagonist with a sample of the subject and measuring a FAM19A1 protein level or a FAM19A1 mRNA level in the sample.

The term “abnormality in a central nervous system function” refers to the inability (or reduced ability) to carry out one or more functions associated with the CNS. In some aspects, central nervous system function comprises a limbic system related function, olfactory system related function, sensory system related function, visual system related function, or combinations thereof.

As described elsewhere in the present disclosure, many diseases or disorders affecting the CNS are associated with some degree of impaired CNS function (e.g., primary symptom associated with glaucoma is impaired vision). Accordingly, in some aspects, a method disclosed herein can also be used to diagnose and/or identify a subject with a disease or disorder associated with the CNS. Non-limiting examples of such a disease or disorder include glaucoma, neuropathic pain, addiction, arachnoid cysts, attention deficit/hyperactivity disorder (ADHD), autism, bipolar disorder, catalepsy, depression, encephalitis, epilepsy/seizures, Locked-in syndrome, meningitis, migraine, multiple sclerosis, myelopathy, Alzheimer's disease, Huntington's disease, Parkinson's disease, Amyotrophic lateral sclerosis (ALS), Batten disease, Tourette's syndrome, traumatic brain injury, post-traumatic stress disorder (PTSD), cerebrospinal damage, stroke, tremors (essential or Parkinsonian), dystonia, schizophrenia, intellectual disabilities, and brain tumor. In certain aspects, abnormality in a central nervous system function is associated with a glaucoma or a neuropathic pain.

In some aspects, a method of diagnosing and/or identifying a subject with an abnormality in a central nervous system function comprises administering a FAM19A1 antagonist to the subject prior to the measuring, such that the contacting between the FAM19A1 antagonist and FAM19A1 occurs in vivo. In some aspects, both the contacting and the measuring is performed in vitro.

In some aspects, an abnormality in a CNS function is associated with an increase in the FAM19A1 protein level and/or in the FAM19A1 mRNA level in the sample compared to a reference (e.g., corresponding value in a sample of a subject who does not suffer from an abnormality in a central nervous system function, e.g., a healthy subject). In certain aspects, the FAM19A1 protein level and/or the FAM19A1 mRNA level is increased by at least about 5%, at least about 10%, at least about 20%, at least about 30%, at least about 40%, at least about 50%, at least about 60%, at least about 70%, at least about 80%, or at least about 90% or more, compared to the reference.

In some aspects, a sample of a subject with an abnormality in a central nervous system function has at least 1.1 fold, at least 1.2 fold, at least 1.3 fold, at least 1.4 fold, at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 15 fold, at least 20 fold, at least 25 fold, or at least 30 fold increase in the FAM19A1 protein level and/or FAM19A1 mRNA level compared to a reference (e.g., corresponding value in a sample of a subject who does not suffer from an abnormality in a central nervous system function, e.g., a healthy subject).

In some aspects, an abnormality in a CNS function is associated with a decrease in the FAM19A1 protein level and/or in the FAM19A1 mRNA level in the sample compared to a reference (e.g., corresponding value in a sample of a subject who does not suffer from an abnormality in a central nervous system function, e.g., a healthy subject). In certain aspects, the FAM19A1 protein level and/or FAM19A1 mRNA level is decreased by at least about 5%, at least about 10%, at least about 20%, at least about 30%, at least about 40%, at least about 50%, at least about 60%, at least about 70%, at least about 80%, or at least about 90% or more, compared to the reference.

In some aspects, a sample of a subject with an abnormality in a central nervous system function has at least 1.1 fold, at least 1.2 fold, at least 1.3 fold, at least 1.4 fold, at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 15 fold, at least 20 fold, at least 25 fold, or at least 30 fold decrease in the FAM19A1 protein level and/or FAM19A1 mRNA level compared to a reference (e.g., corresponding value in a sample of a subject who does not suffer from an abnormality in a central nervous system function, e.g., a healthy subject).

In some aspects, a FAM19A1 protein level is measured by an immunohistochemistry, Western blot, radioimmunoassay, enzyme linked immunosorbent assay (ELISA), radioimmunodiffusion, immunoprecipitation assay, Ouchterlony immunodiffusion method, rocket immunoelectrophoresis, tissue immunostaining method, complement fixation assay, FACS, protein chip, or combinations thereof. In some aspects, a FAM19A1 mRNA level is measured by a reverse transcription polymerase chain reaction (RT-PCR), a real time polymerase chain reaction, a Northern blot, or combinations thereof. In some aspects, a FAM19A1 protein level is measured by an assay using a FAM19A1 antagonist disclosed herein (e.g., anti-FAM19A1 antibody).

In some aspects, a sample (e.g., in which the FAM19A1 protein level and/or the FAM19A1 mRNA level is measured) comprises a tissue, cell, blood, serum, plasma, saliva, urine, cerebral spinal fluid (CSF), or combinations thereof.

In some aspects, a method of diagnosing and/or identifying a subject with an abnormality in a central nervous system function further comprises administering a FAM19A1 antagonist to the subject if the FAM19A1 protein level and/or FAM19A1 mRNA level is increased compared to a reference (e.g., corresponding value in a sample of a subject who does not suffer from an abnormality in a central nervous system function, e.g., a healthy subject).

In some aspects, a method of diagnosing and/or identifying a subject with an abnormality in a central nervous system function further comprises administering a FAM19A1 agonist to the subject if the FAM19A1 protein level and/or FAM19A1 mRNA level is decreased compared to a reference (e.g., corresponding value in a sample of a subject who does not suffer from an abnormality in a central nervous system function, e.g., a healthy subject).

In some aspects, a FAM19A1 agonist comprises a FAM19A1 protein. In some aspects, a FAM19A1 antagonist comprises an anti-FAM19A1 antibody, a polynucleotide encoding the anti-FAM19A1 antibody, a vector comprising the polynucleotide thereof, a cell comprising the polynucleotide thereof, or any combination thereof. In some aspects, a FAM19A1 antagonist comprises an antisense oligonucleotide, siRNA, shRNA, miRNA, dsRNA, aptamer, PNA that specifically targets FAM19A1, or a vector including the same. In certain aspects, a FAM19A1 antagonist is an anti-FAM19A1 antibody.

### III. FAM19A1 Antagonists

One or more FAM19A1 antagonists can be used with the present methods. In some aspects, a FAM19A1 antagonist is an antisense oligonucleotide, siRNA, shRNA, miRNA, dsRNA, aptamer, PNA (peptide nucleic acid) that specifically targets FAM19A1, or a vector including the same. In some aspects, a FAM19A1 antagonist is anti-FAM19A1 antibody, a polynucleotide encoding the anti-FAM19A1 antibody, or a vector comprising the polynucleotide thereof.

Antibodies that are useful in the methods disclosed herein include monoclonal antibodies, which are characterized by particular functional features or properties. For example, the antibodies specifically bind human FAM19A1, including soluble FAM19A1 and membrane bound FAM19A1. In addition to binding specifically to soluble and/or membrane bound human FAM19A1, the antibodies described herein also (a) binds to soluble human FAM19A1 with a K_{D} of 10 nM or less; (b) binds to membrane bound human FAM19A1 with a K_{D} of 10 nM or less; or both (a) and (b).

In some aspects, an anti-FAM19A1 antibody disclosed herein specifically binds to soluble human FAM19A1 or membrane-bound human FAM19A1 with high affinity, for example, with a K_{D} of 10⁻⁷ M or less, 10⁻⁸ M (10 nM) or less, 10⁻⁹ M (1 nM) or less, 10⁻¹⁰ M (0.1 nM) or less, 10⁻¹¹ M or less, or 10⁻¹² M or less, e.g., 10⁻¹² M to 10⁻⁷ M, 10⁻¹¹ M to 10⁻⁷ M, 10⁻¹⁰ M to 10⁻⁷ M, or 10⁻⁹ M to 10⁻⁷ M, e.g., 10⁻¹² M, 5×10⁻¹² M, 10⁻¹¹ M, 5×10⁻¹¹ M, 10⁻¹⁰ M, 5×10⁻¹⁰ M, 10⁻⁹ M, 5×10⁻⁹ M, 10⁻⁸ M, 5×10⁻⁸ M, 10⁻⁷ M, or 5×10⁻⁷ M. Standard assays to evaluate the binding ability of the antibody toward human FAM19A1 of various species are known in the art, including for example, ELISAs, Western blots, and RIAs. Suitable assays are described in detail in the Examples. The binding kinetics (e.g., binding affinity) of the antibodies also can be assessed by standard assays known in the art, such as by ELISA, BIACORE™ analysis or KINEXA®.

In some aspects, an anti-FAM19A1 antibody disclosed herein binds to soluble human FAM19A1 with a K_{D}, e.g., as determined by ELISA, of 10⁻⁷ M or less, 10⁻⁸ M (10 nM) or less, 10⁻⁹ M (1 nM) or less, 10⁻¹⁰ M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, or 10⁻⁸ M to 10⁻⁷ M. In some aspects, an anti-FAM19A1 antibody binds to soluble FAM19A1 with a K_{D} of 10 nM or less, e.g., between 0.1 and 10 nM, between 0.1 and 5 nM, between 0.1 and 1 nM, between 0.5 and 10 nM, between 0.5 and 5 nM, between 0.5 and 1 nM, between 1 and 10 nM, between 1 and 5 nM, or between 5 and 10 nM. In certain aspects, an anti-FAM19A1 antibody specifically binds to soluble human FAM19A1 with a K_{D} of about 1 pM, 2 pM, 3 pM, 4 pM, 5 pM, 6 pM, 7 pM, 8 pM, 9 pM, 10 pM, 20 pM, 30 pM, 40 pM, 50 pM, 60 pM, 70 pM, 80 pM, 90 pM, 100 pM, 200 pM, 300 pM, 400 pM, 500 pM, 600 pM, 700 pM, 800 pM, or 900 pM, or about 1 nM, 2 nM, 3 nM, 4 nM, 5 nM, 6 nM, 7 nM, 8 nM, or 9 nM, or about 10 nM, 20 nM, 30 nM, 40 nM, 50 nM, 60 nM, 70 nM, 80 nM, or 90 nM, as determined by as determined by ELISA.

In some aspects, an anti-FAM19A1 antibody binds to membrane-bound human FAM19A1 with a K_{D}, e.g., as determined by ELISA, of 10⁻⁷ M or less, 10⁻⁸ M (10 nM) or less, 10⁻⁹ M (1 nM) or less, 10⁻¹⁰ M or less, 10⁻¹² M to 10⁻⁷ M, 10⁻¹¹ M to 10⁻⁷ M, 10⁻¹⁰ M to 10⁻⁷ M, 10⁻⁹ M to 10⁻⁷ M, or 10⁻⁸ M to 10⁻⁷ M. In certain aspects, an anti-FAM19A1 antibody specifically binds to membrane-bound human FAM19A1 with a K_{D} of 10 nM or less as determined by ELISA, e.g., between 0.1 and 10 nM, between 0.1 and 5 nM, between 0.1 and 1 nM, between 0.5 and 10 nM, between 0.5 and 5 nM, between 0.5 and 1 nM, between 1 and 10 nM, between 1 and 5 nM, or between 5 and 10 nM. In some aspects, the anti-FAM19A1 antibody or antigen-binding portion thereof binds to membrane-bound human FAM19A1 with a K_{D} of about 1 pM, 2 pM, 3 pM, 4 pM, 5 pM, 6 pM, 7 pM, 8 pM, 9 pM, 10 pM, 20 pM, 30 pM, 40 pM, 50 pM, 60 pM, 70 pM, 80 pM, 90 pM, 100 pM, 200 pM, 300 pM, 400 pM, 500 pM, 600 pM, 700 pM, 800 pM, or 900 pM, or about 1 nM, 2 nM, 3 nM, 4 nM, 5 nM, 6 nM, 7 nM, 8 nM, or 9 nM, or about 10 nM, 20 nM, 30 nM, 40 nM, 50 nM, 60 nM, 70 nM, 80 nM, or 90 nM, as determined by as determined by ELISA.

In addition to the above, FAM19A1 antagonists (e.g., anti-FAM19A1 antibody) exhibits one or more of the following functional properties:
(1) promotes the differentiation of neurons;
(2) increases neurite outgrowth in differentiated neurons;
(3) reduces, reverses, and/or prevents one or more symptoms associated with glaucoma;
(4) improves retinal potential (e.g., as evidenced by increased oscillatory potential);
(5) reduces and/or restores the loss of retinal ganglion cells (e.g., observed in glaucoma subjects);
(6) reduces, reverses, and/or prevents one or more symptoms associated with neuropathic pain;
(7) increases latency and/or threshold to an external stimulus; and
(8) increases and/or regulates sensory nerve conduction velocity.

Other functional properties of the anti-FAM19A1 antibodies disclosed herein are provided throughout the application.

In some aspects, an anti-FAM19A1 antibody disclosed herein cross-competes for binding to (or inhibits binding of) a human FAM19A1 epitope with an anti-FAM19A1 antibody comprising CDRs or variable regions disclosed herein (e.g., 1C1, 1A11, 2G7, and 3A8).

In some aspects, anti-FAM19A1 antibody of the present disclosure inhibits binding of a reference antibody comprising heavy chain CDR1, CDR2, and CDR3, and light chain CDR1, CDR2, and CDR3, (i) wherein the heavy chain CDR1, CDR2, and CDR3 of the reference antibody comprise the amino acid sequence set forth in SEQ ID NOs: 10-12, respectively, and light chain CDR1, CDR2, and CDR3 of the reference antibody comprise the amino acid sequence set forth in SEQ ID NOs: 13-15, respectively; (ii) wherein the heavy chain CDR1, CDR2, and CDR3 of the reference antibody comprise the amino acid sequence set forth in SEQ ID NOs: 4-6, respectively, and light chain CDR1, CDR2, and CDR3 of the reference antibody comprise the amino acid sequence set forth in SEQ ID NOs: 7-9, respectively; (iii) wherein the heavy chain CDR1, CDR2, and CDR3 of the reference antibody comprise the amino acid sequence set forth in SEQ ID NOs: 16-18, respectively, and light chain CDR1, CDR2, and CDR3 of the reference antibody comprise the amino acid sequence set forth in SEQ ID NO: 19-21, respectively; or (iv) wherein the heavy chain CDR1, CDR2, and CDR3 of the reference antibody comprise the amino acid sequence set forth in SEQ ID NO: 22-24, respectively, and light chain CDR1, CDR2, and CDR3 of the reference antibody comprise the amino acid sequence set forth in SEQ ID NO: 25-27, respectively.

In some aspects, the reference antibody comprises (a) heavy and light chain variable regions comprising the amino acid sequence set forth in SEQ ID NOs: 30 and 31, respectively; (b) heavy and light chain variable regions comprising the amino acid sequence set forth in SEQ ID NOs: 28 and 29, respectively; (c) heavy and light chain variable regions comprising the amino acid sequence set forth in SEQ ID NOs: 32 and 33, respectively; or (d) heavy and light chain variable regions comprising the amino acid sequence set forth in SEQ ID NOs: 34 and 35, respectively.

In some aspects, an anti-FAM19A1 antibody disclosed herein inhibits binding of such a reference antibody to human FAM19A1 by at least 10%, 20%, 30%, 40%, 50%, 60%, 70%, 80%, 90%, or by 100%. Competing antibodies bind to the same epitope, an overlapping epitope, or to adjacent epitopes (e.g., as evidenced by steric hindrance). Whether two antibodies compete with each other for binding to a target can be determined using competition experiments known in the art such as RIA and EIA.

In some aspects, an anti-FAM19A1 antibody binds to the same FAM19A1 epitope as a reference antibody disclosed herein comprising a heavy chain CDR1, CDR2, and CDR3 and a light chain CDR1, CDR2, and CDR3, (i) wherein the heavy chain CDR1 comprises the amino acid sequence set forth in SEQ ID NO: 10, the heavy chain CDR2 comprises the amino acid sequence set forth in SEQ ID NO: 11, and the heavy chain CDR3 comprises the amino acid sequence set forth in SEQ ID NO: 12, the light chain CDR1 comprises the amino acid sequence set forth in SEQ ID NO: 13, the light chain CDR2 comprises the amino acid sequence set forth in SEQ ID NO: 14, and the light chain CDR3 comprises the amino acid sequence set forth in SEQ ID NO: 15; (ii) wherein the heavy chain CDR1 comprises the amino acid sequence set forth in SEQ ID NO: 4, the heavy chain CDR2 comprises the amino acid sequence set forth in SEQ ID NO: 5, and the heavy chain CDR3 comprises the amino acid sequence set forth in SEQ ID NO: 6, the light chain CDR1 comprises the amino acid sequence set forth in SEQ ID NO: 7, the light chain CDR2 comprises the amino acid sequence set forth in SEQ ID NO: 8, and the light chain CDR3 comprises the amino acid sequence set forth in SEQ ID NO: 9; (iii) wherein the heavy chain CDR1 comprises the amino acid sequence set forth in SEQ ID NO: 16, the heavy chain CDR2 comprises the amino acid sequence set forth in SEQ ID NO: 17, and the heavy chain CDR3 comprises the amino acid sequence set forth in SEQ ID NO: 18, the light chain CDR1 comprises the amino acid sequence set forth in SEQ ID NO: 19, the light chain CDR2 comprises the amino acid sequence set forth in SEQ ID NO: 20, and the light chain CDR3 comprises the amino acid sequence set forth in SEQ ID NO: 21; or (iv) wherein the heavy chain CDR1 comprises the amino acid sequence set forth in SEQ ID NO: 22, the heavy chain CDR2 comprises the amino acid sequence set forth in SEQ ID NO: 23, and the heavy chain CDR3 comprises the amino acid sequence set forth in SEQ ID NO: 24, the light chain CDR1 comprises the amino acid sequence set forth in SEQ ID NO: 25, the light chain CDR2 comprises the amino acid sequence set forth in SEQ ID NO: 26, and the light chain CDR3 comprises the amino acid sequence set forth in SEQ ID NO: 27. In certain aspects, the reference antibody comprises: (i) a heavy chain variable domain comprising the amino acid sequence set forth in SEQ ID NOs: 30, 28, 32, or 34, and (ii) a light chain variable domain comprising the amino acid sequence set forth in SEQ ID NOs: 31, 29, 33, or 35.

In some aspects, the reference antibody comprises (a) heavy and light chain variable regions comprising the amino acid sequence set forth in SEQ ID NOs: 30 and 31, respectively; (b) heavy and light chain variable regions comprising the amino acid sequence set forth in SEQ ID NOs: 28 and 29, respectively; (c) heavy and light chain variable regions comprising the amino acid sequence set forth in SEQ ID NOs: 32 and 33, respectively; or (d) heavy and light chain variable regions comprising the amino acid sequence set forth in SEQ ID NOs: 34 and 35, respectively.

Techniques for determining whether two antibodies bind to the same epitope include, e.g., epitope mapping methods, such as, x-ray analyses of crystals of antigen:antibody complexes which provides atomic resolution of the epitope and hydrogen/deuterium exchange mass spectrometry (HDX-MS), methods monitoring the binding of the antibody to antigen fragments or mutated variations of the antigen, where loss of binding due to a modification of an amino acid residue within the antigen sequence is often considered an indication of an epitope component, computational combinatorial methods for epitope mapping.

An anti-FAM19A1 antibody of the present disclosure can bind to at least one epitope of mature human FAM19A1, as determined, e.g., by binding of the antibodies to fragments of human FAM19A1. In some aspects, an anti-FAM19A1 antibody binds to at least one epitope selected from the group consisting of D112, M117, A119, T120, N122, and combinations thereof.

In some aspects, provided herein is an anti-FAM19A1 antibody that binds to FAM19A1 with a 20%, 25%, 30%, 35%, 40%, 45%, 50%, 55%, 60%, 65%, 70%, 75%, 80%, 85%, 90%, 95% or higher affinity than to another protein in the FAM19A family as measured by, e.g., an immunoassay (e.g., ELISA), surface plasmon resonance, or kinetic exclusion assay. In certain aspects, an anti-FAM19A1 antibody disclosed herein has no cross reactivity with another protein in the FAM19A family as measured by, e.g., an immunoassay (e.g., ELISA), surface plasmon resonance, or kinetic exclusion assay.

In some aspects, an anti-FAM19A1 antibody is not a native antibody or is not a naturally-occurring antibody. For example, in certain aspects, an anti-FAM19A1 antibody of the present disclosure has post-translational modifications that are different from those of antibodies that are naturally-occurring, such as by having more, less or a different type of post-translational modification.

### IV. Exemplary Anti-FAM19A1 Antibodies

Particular antibodies that can be used in the methods disclosed herein are antibodies, e.g., monoclonal antibodies, having the CDR and/or variable region sequences disclosed herein, as well as antibodies having at least 80% identity (e.g., at least 85%, at least 90%, at least 95%, or at least 99% identity) to their variable region or CDR sequences. The VH and VL amino acid sequences of different anti-FAM19A1 antibodies of the present disclosure are provided in Tables 6 and 7, respectively

TABLE 4
Variable heavy chain CDR amino acid sequences (according to IMGT system)

| Antibody | VH-CDR1 | VH-CDR2 | VH-CDR3 |
|---|---|---|---|
| Anti-FAM19A1 | GFAFSDYG (SEQ | ISYDGSKR (SEQ | ARPGDYALAFDL (SEQ ID NO: |
| (“1C1”) | ID NO: 10) | ID NO: 11) | 12) |
| Anti-FAM19A1 | GYSFTGYD (SEQ | MNPSSGNT (SEQ | ARALNSVYYYHALDV (SEQ ID |
| (“1A11”) | ID NO: 4) | ID NO: 5) | NO: 6) |
| Anti-FAM19A1 | GFPFGDHA (SEQ | IRSNTYGGTT (SEQ | TKDITGGGFWSDYGDYFDAFDF |
| (“2G7”) | ID NO: 16) | ID NO: 17) | (SEQ ID NO: 18) |
| Anti-FAM19A1 | GDAITSGSYY | ISHSGST (SEQ ID | AGDTALVGAYSI (SEQ ID NO: |
| (“3A8”) | (SEQ ID NO: 22) | NO: 23) | 24) |

TABLE 5
Variable light chain CDR amino acid sequences (according to IMGT system)

| Antibody | VL-CDR1 | VL-CDR2 | VL-CDR3 |
|---|---|---|---|
| Anti-FAM19A1 | SGSVSTSHF (SEQ | SRN (SEQ ID NO: | ALYMGRGNWV (SEQ ID NO: |
| (“1C1”) | ID NO: 13) | 14) | 15) |
| Anti-FAM19A1 | RLHNKY (SEQ ID | QDA (SEQ ID NO: | QTWDRSTGV (SEQ ID NO: 9) |
| (“1A11”) | NO: 7) | 8) | |
| Anti-FAM19A1 | KLGYKY (SEQ ID | QDK (SEQ ID NO: | QAWDSGTASHV (SEQ ID NO: |
| (“2G7”) | NO: 19) | 20) | 21) |
| Anti-FAM19A1 | NLRTKY (SEQ ID | QDT (SEQ ID NO: | MTWDVDTTSMI (SEQ ID NO: |
| (“3A8”) | NO: 25) | 26) | 27) |

TABLE 6
Variable heavy chain amino acid sequence

| Antibody | VH Amino Acid Sequence (SEQ ID NO) |
|---|---|
| Anti-FAM19A1 | QVQLVESGGGVAQPGRSLRLSCAASGFAFSDYGIHWVRQAPGKGLEWVALIS |
| (“1C1”) | YDGSKRSYADSVKGRFAISRDNSKNTLYLQMNSLRAEDTAVYYCARPGDYAL |
| | AFDLWGQGTMVTVSS (SEQ ID NO: 30) |
| Anti-FAM19A1 | QVQLVESGAEVRKPGASVKVSCKASGYSFTGYDINWVRQAPGQGLEWMGWMN |
| (“1A11”) | PSSGNTGYAQKFQGRVTMTRDSSISTAYMELSSLRSEDTAVYYCARALNSVY |
| | YYHALDVWGQGTTITVSS (SEQ ID NO: 28) |
| Anti-FAM19A1 | QVQLVESGGGLVKPGRSLSLSCTTSGFPFGDHAINWVRQAPGKGLEWVGFIR |
| (“2G7”) | SNTYGGTTQYAASVEGRFTISRDDSKSIAYLQMNSLRAEDTAVYYCTKDITG |
| | GGFWSDYGDYFDAFDFWGQGTMVTVSS (SEQ ID NO: 32) |
| Anti-FAM19A1 | QVQLQQSGPGLVKPSETLSLICSVSGDAITSGSYYWGWIRQSPGRGLEWIGE |
| (“3A8”) | ISHSGSTDYNPSLKSRVTISVDKSRNQFSLRLNSVTAVDTAVYYCAGDTALV |
| | GAYSIWGQGTMVTVSS (SEQ ID NO: 34) |

TABLE 7
Variable light chain amino acid sequence

| Antibody | VL Amino Acid Sequence (SEQ ID NO) |
|---|---|
| Anti-FAM19A1 | QTVVTQEPSFSVSPGETVTLTCGLSSGSVSTSHFPSWYRQTPGQAPRPLIDS |
| (“1C1”) | RNARSIGVPDRFSGSIVGTKATLTISGAQAEDECNYYCALYMGRGNWVFGGG |
| | TKLTVL (SEQ ID NO: 31) |
| Anti-FAM19A1 | SYELTQPPSASVSPGQTASITCSGDRLHNKYTSWYQQKPGQSPLLVIYQDAK |
| (“1A11”) | RPSGIPERFSGSSSRGTATLTISGTQATDEADYYCQTWDRSTGVFGTGTKVT |
| | VL (SEQ ID NO: 29) |
| Anti-FAM19A1 | SYELTQPLSVSVSPGQTASISCSGDKLGYKYASWYQQKPGQSPVVVIYQDKK |
| (“2G7”) | RPSGIPERFSGSNSGNTATLTISGTQPMDEADYYCQAWDSGTASHVFGTGTK |
| | VTVL (SEQ ID NO: 33) |
| Anti-FAM19A1 | SYELTQAPSLSVSPGQTANIICSGDNLRTKYVSWYQQKPGQSPLLVIYQDTR |
| (“3A8”) | RPSGIPARFSGSNSGNTATLTISGTQTRDESTYYCMTWDVDTTSMIFGGGTK |
| | LTVL (SEQ ID NO: 35) |

In some aspects, an anti-FAM19A1 antibody of the present disclosure comprises heavy and light chain variable regions, wherein the heavy chain variable region comprises the amino acid sequence set forth in SEQ ID NO: 30, 28, 32, or 34. In some aspects, an anti-FAM19A1 antibody disclosed herein comprises the CDRs of the heavy chain variable region selected from the group consisting of SEQ ID NOs: 30, 28, 32, and 34.

In some aspects, an anti-FAM19A1 antibody disclosed herein comprises heavy and light chain variable regions, wherein the light chain variable region comprises the amino acid sequence set forth in SEQ ID NO: 31, 29, 33, or 35. In some aspects, an anti-FAM19A1 antibody disclosed herein comprises the CDRs of the light chain variable region selected from the group consisting of SEQ ID NOs: 31, 29, 33, and 35.

In some aspects, an anti-FAM19A1 antibody comprises CDRs of the heavy chain variable region selected from the group consisting of SEQ ID NOs: 30, 28, 32, and 34 and CDRs of the light chain variable region selected from the group consisting of SEQ ID NOs: 31, 29, 33, and 35.

In some aspects, an anti-FAM19A1 antibody comprises heavy and light chain variable regions, (i) wherein the heavy chain variable region comprises the amino acid sequence set forth in SEQ ID NO: 30 and wherein the light chain variable region comprises the amino acid sequence set forth in SEQ ID NO: 31; (ii) wherein the heavy chain variable region comprises the amino acid sequence set forth in SEQ ID NO: 28 and wherein the light chain variable region comprises the amino acid sequence set forth in SEQ ID NO: 29; (iii) wherein the heavy chain variable region comprises the amino acid sequence set forth in SEQ ID NO: 32 and wherein the light chain variable region comprises the amino acid sequence set forth in SEQ ID NO: 33; and (iv) wherein the heavy chain variable region comprises the amino acid sequence set forth in SEQ ID NO: 34 and wherein the light chain variable region comprises the amino acid sequence set forth in SEQ ID NO: 35.

In some aspects, an anti-FAM19A1 antibody comprises heavy and light chain variable regions, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 30, 28, 32, or 34.

In some aspects, an anti-FAM19A1 antibody comprises heavy and light chain variable regions, wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 31, 29, 33, or 35.

In some aspects, an anti-FAM19A1 antibody comprises heavy and light chain variable regions, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 30, 28, 32, or 34, and wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 31, 29, 33, or 35.

In some aspects, an anti-FAM19A1 antibody comprises:
(a) heavy and light chain variable regions comprising the amino acid sequence set forth in SEQ ID NOs: 30 and 31, respectively;
(b) heavy and light chain variable regions comprising the amino acid sequence set forth in SEQ ID NOs: 28 and 29, respectively;
(c) heavy and light chain variable regions comprising the amino acid sequence set forth in SEQ ID NOs: 32 and 33, respectively; or
(d) heavy and light chain variable regions comprising the amino acid sequence set forth in SEQ ID NOs: 34 and 35, respectively.

In some aspects, an anti-FAM19A1 antibody of the present disclosure comprises (i) the heavy chain CDR1, CDR2 and CDR3 of 1C1, or combinations thereof, and/or the light chain CDR1, CDR2, and CDR3 of 1C1, or any combinations thereof; (ii) the heavy chain CDR1, CDR2 and CDR3 of 1A11, or combinations thereof, and/or the light chain CDR1, CDR2, and CDR3 of 1A11, or any combinations thereof; (iii) the heavy chain CDR1, CDR2 and CDR3 of 2G7, or combinations thereof, and/or the light chain CDR1, CDR2, and CDR3 of 2G7, or any combinations thereof; or (iv) the heavy chain CDR1, CDR2 and CDR3 of 3A8, or combinations thereof, and/or the light chain CDR1, CDR2, and CDR3 of 3A8, or any combinations thereof. The amino acid sequences of the VH CDR1, CDR2, and CDR3 for the different anti-FAM19A1 antibodies disclosed herein are provided in Table 4. The amino acid sequences of the VL CDR1, CDR2, and CDR3 for the different anti-FAM19A1 antibodies disclosed herein are provided in Table 5.

In some aspects, an anti-FAM19A1 antibody, which specifically binds to human FAM19A1, comprises:
(a) a VH CDR1 comprising the amino acid sequence set forth in SEQ ID NO: 10;
(b) a VH CDR2 comprising the amino acid sequence set forth in SEQ ID NO: 11; and/or
(c) a VH CDR3 comprising the amino acid sequence set forth in SEQ ID NO: 12.

In some aspects, an antibody comprises one, two, or all three of the VH CDRs above.

In some aspects, an anti-FAM19A1 antibody, which specifically binds to human FAM19A1, comprises:
(a) a VL CDR1 comprising the amino acid sequence set forth in SEQ ID NO: 13;
(b) a VL CDR2 comprising the amino acid sequence set forth in SEQ ID NO: 14; and/or
(c) a VL CDR3 comprising the amino acid sequence set forth in SEQ ID NO: 15.

In some aspects, an antibody comprises one, two, or all three of the VL CDRs above.

In some aspects, an anti-FAM19A1 antibody, which specifically binds to human FAM19A1, comprises:
(a) a VH CDR1 comprising the amino acid sequence set forth in SEQ ID NO: 10;
(b) a VH CDR2 comprising the amino acid sequence set forth in SEQ ID NO: 11;
(c) a VH CDR3 comprising the amino acid sequence set forth in SEQ ID NO: 12;
(d) a VL CDR1 comprising the amino acid sequence set forth in SEQ ID NO: 13;
(e) a VL CDR2 comprising the amino acid sequence set forth in SEQ ID NO: 14; and/or
(f) a VL CDR3 comprising the amino acid sequence set forth in SEQ ID NO: 15.

In some aspects, an anti-FAM19A1 antibody, which specifically binds to human FAM19A1, comprises:
(a) a VH CDR1 comprising the amino acid sequence set forth in SEQ ID NO: 4;
(b) a VH CDR2 comprising the amino acid sequence set forth in SEQ ID NO: 5; and/or
(c) a VH CDR3 comprising the amino acid sequence set forth in SEQ ID NO: 6.

In some aspects, an antibody comprises one, two, or all three of the VH CDRs above.

In some aspects, an anti-FAM19A1 antibody, which specifically binds to human FAM19A1, comprises:
(a) a VL CDR1 comprising the amino acid sequence set forth in SEQ ID NO: 7;
(b) a VL CDR2 comprising the amino acid sequence set forth in SEQ ID NO: 8; and/or
(c) a VL CDR3 comprising the amino acid sequence set forth in SEQ ID NO: 9.

In some aspects, an antibody comprises one, two, or all three of the VL CDRs above.

In some aspects, an anti-FAM19A1 antibody, which specifically binds to human FAM19A1, comprises:
(a) a VH CDR1 comprising the amino acid sequence set forth in SEQ ID NO: 4;
(b) a VH CDR2 comprising the amino acid sequence set forth in SEQ ID NO: 5;
(c) a VH CDR3 comprising the amino acid sequence set forth in SEQ ID NO: 6;
(d) a VL CDR1 comprising the amino acid sequence set forth in SEQ ID NO: 7;
(e) a VL CDR2 comprising the amino acid sequence set forth in SEQ ID NO: 8; and/or
(f) a VL CDR3 comprising the amino acid sequence set forth in SEQ ID NO: 9.

In some aspects, an anti-FAM19A1 antibody, which specifically binds to human FAM19A1, comprises:
(a) a VH CDR1 comprising the amino acid sequence set forth in SEQ ID NO: 16;
(b) a VH CDR2 comprising the amino acid sequence set forth in SEQ ID NO: 17; and/or
(c) a VH CDR3 comprising the amino acid sequence set forth in SEQ ID NO: 18.

In some aspects, an antibody comprises one, two, or all three of the VH CDRs above.

In some aspects, an anti-FAM19A1 antibody, which specifically binds to human FAM19A1, comprises:
(a) a VL CDR1 comprising the amino acid sequence set forth in SEQ ID NO: 19;
(b) a VL CDR2 comprising the amino acid sequence set forth in SEQ ID NO: 20; and/or
(c) a VL CDR3 comprising the amino acid sequence set forth in SEQ ID NO: 21.

In some aspects, an antibody comprises one, two, or all three of the VL CDRs above.

In some aspects, an anti-FAM19A1 antibody, which specifically binds to human FAM19A1, comprises:
(a) a VH CDR1 comprising the amino acid sequence set forth in SEQ ID NO: 16;
(b) a VH CDR2 comprising the amino acid sequence set forth in SEQ ID NO: 17;
(c) a VH CDR3 comprising the amino acid sequence set forth in SEQ ID NO: 18;
(d) a VL CDR1 comprising the amino acid sequence set forth in SEQ ID NO: 19;
(e) a VL CDR2 comprising the amino acid sequence set forth in SEQ ID NO: 20; and/or
(f) a VL CDR3 comprising the amino acid sequence set forth in SEQ ID NO: 21.

In some aspects, an anti-FAM19A1 antibody, which specifically binds to human FAM19A1, comprises:
(a) a VH CDR1 comprising the amino acid sequence set forth in SEQ ID NO: 22;
(b) a VH CDR2 comprising the amino acid sequence set forth in SEQ ID NO: 23; and/or
(c) a VH CDR3 comprising the amino acid sequence set forth in SEQ ID NO: 24.

In some aspects, an antibody comprises one, two, or all three of the VH CDRs above.

In some aspects, an anti-FAM19A1 antibody, which specifically binds to human FAM19A1, comprises:
(a) a VL CDR1 comprising the amino acid sequence set forth in SEQ ID NO: 25;
(b) a VL CDR2 comprising the amino acid sequence set forth in SEQ ID NO: 26; and/or
(c) a VL CDR3 comprising the amino acid sequence set forth in SEQ ID NO: 27.

In some aspects, an antibody comprises one, two, or all three of the VL CDRs above.

In some aspects, an anti-FAM19A1 antibody, which specifically binds to human FAM19A1, comprises:
(a) a VH CDR1 comprising the amino acid sequence set forth in SEQ ID NO: 22;
(b) a VH CDR2 comprising the amino acid sequence set forth in SEQ ID NO: 23;
(c) a VH CDR3 comprising the amino acid sequence set forth in SEQ ID NO: 24;
(d) a VL CDR1 comprising the amino acid sequence set forth in SEQ ID NO: 25;
(e) a VL CDR2 comprising the amino acid sequence set forth in SEQ ID NO: 26; and/or
(f) a VL CDR3 comprising the amino acid sequence set forth in SEQ ID NO: 27.

A VH domain, or one or more CDRs thereof, described herein can be linked to a constant domain for forming a heavy chain, e.g., a full length heavy chain. Similarly, a VL domain, or one or more CDRs thereof, described herein can be linked to a constant domain for forming a light chain, e.g., a full length light chain. A full length heavy chain and full length light chain combine to form a full length antibody.

Accordingly, in some aspects, the anti-FAM19A1 antibody comprises an antibody light chain and heavy chain, e.g., a separate light chain and heavy chain. With respect to the light chain, in certain aspects, the light chain of an antibody described herein is a kappa light chain. In some aspects, the light chain of an antibody described herein is a lambda light chain. In some aspects, the light chain of an antibody described herein is a human kappa light chain or a human lambda light chain. In certain aspects, an antibody described herein, which specifically binds to a FAM19A1 polypeptide (e.g., human FAM19A1) comprises a light chain which comprises any VL or VL CDR amino acid sequences described herein, and wherein the constant region of the light chain comprises the amino acid sequence of a human kappa light chain constant region. In some aspects, an antibody described herein, which specifically binds to a FAM19A1 polypeptide (e.g., human FAM19A1) comprises a light chain which comprises a VL or VL CDR amino acid sequences described herein, and wherein the constant region of the light chain comprises the amino acid sequence of a human lambda light chain constant region. Non-limiting examples of human constant region sequences have been described in the art, e.g., see U.S. Pat. No. 5,693,780 and Kabat E A et al, (1991) supra.

With respect to the heavy chain, in some aspects, the heavy chain of an antibody described herein can be an alpha (a), delta (8), epsilon (e), gamma (γ) or mu (p) heavy chain. In some aspects, the heavy chain of an antibody described can comprise a human alpha (a), delta (8), epsilon (e), gamma (γ) or mu (p) heavy chain. In some aspects, an antibody described herein, which specifically binds to FAM19A1 (e.g., human FAM19A1), comprises a heavy chain which comprises a VH or VH CDR amino acid sequence described herein, and wherein the constant region of the heavy chain comprises the amino acid sequence of a human gamma (γ) heavy chain constant region. In some aspects, an antibody described herein, which specifically binds to FAM19A1 (e.g., human FAM19A1), comprises a heavy chain which comprises a VH or VH CDR amino acid sequence disclosed herein, and wherein the constant region of the heavy chain comprises the amino acid of a human heavy chain described herein or known in the art. Non-limiting examples of human constant region sequences have been described in the art, see. e.g., U.S. Pat. No. 5,693,780 and Kabat E A et al., (1991) supra.

In some aspects, the antibody described herein, which specifically binds to FAM19A1 (e.g., human FAM19A1), comprises a VL domain and a VH domain comprising the VH or VH CDRs and VL and VL CDRs described herein, and wherein the constant regions comprise the amino acid sequences of the constant regions of an IgG, IgE, IgM, IgD, IgA or IgY immunoglobulin molecule, or a human IgG, IgE, IgM, IgD, IgA or IgY immunoglobulin molecule. In certain aspects, an antibody described herein, which specifically binds to FAM19A1 (e.g., human FAM19A1) comprises a VL domain and a VH domain comprising any amino acid sequences described herein, and wherein the constant regions comprise the amino acid sequences of the constant regions of an IgG, IgE, IgM, IgD, IgA or IgY immunoglobulin molecule, any subclass (e.g., IgG1, IgG2, IgG3, IgG4, IgA1 and IgA2) of immunoglobulin molecule. In some aspects, the constant regions comprise the amino acid sequences of the constant regions of a human IgG, which are naturally-occurring, including subclasses (e.g., IgG1, IgG2, IgG3 or IgG4), and allotypes (e.g., G1m, G2m, G3m, and nG4m) and variants thereof. See, e.g., Vidarsson G. et al. Front Immunol. 5:520 (published online Oct. 20, 2014) and Jefferis R. and Lefranc M P, mAbs 1:4, 1-7(2009). In some aspects, the constant regions comprise the amino acid sequences of the constant regions of a human IgG1, IgG2, IgG3, or IgG4, or variants thereof.

In some aspects, an anti-FAM19A1 antibody disclosed herein does not have Fc effector functions, e.g., complement-dependent cytotoxicity (CDC) and/or antibody-dependent cellular phagocytosis (ADCP). Effector functions are mediated by the Fc region and the residues most proximal to the hinge region in the CH2 domain of the Fc region are responsible for effector functions of antibodies as it contains a largely overlapping binding site for C1q (complement) and IgG-Fc receptors (FcγR) on effector cells of the innate immune system. Also, IgG2 and IgG4 antibodies have lower levels of Fc effector functions than IgG1 and IgG3 antibodies. Effector functions of an antibody can be reduced or avoided by different approaches known in the art, including (1) using antibody fragments lacking the Fc region (e.g., such as a Fab, F(ab′)₂, single chain Fv (scFv), or a sdAb consisting of a monomeric VH or VL domain); (2) generating aglycosylated antibodies, which can be generated by, for example, deleting or altering the residue the sugar is attached to, removing the sugars enzymatically, producing the antibody in cells cultured in the presence of a glycosylation inhibitor, or by expressing the antibody in cells unable to glycosylate proteins (e.g., bacterial host cells, see. e.g., U.S. Pub. No. 20120100140); (3) employing Fc regions from an IgG subclass that have reduced effector function (e.g., a Fc region from IgG2 or IgG4 antibodies or a chimeric Fc region comprising a CH2 domain from IgG2 or IgG4 antibodies, see, e.g., U.S. Pub. No. 20120100140 and Lau C. et al. J. Immunol. 191:4769-4777 (2013)); and (4) generating a Fc region with mutations that result in reduced or no Fc functions. See, e.g., U.S. Pub. No. 20120100140 and U.S. and PCT applications cited therein and An et al., mAbs 1:6, 572-579 (2009).

Thus, in some aspects, an antigen-binding fragment disclosed herein is a Fab, a Fab′, a F(ab′)2, a Fv, a single chain Fv (scFv), or a sdAb consisting of a monomeric VH or VL domain. Such antibody fragments are well known in the art and are described supra.

In some aspects, an anti-FAM19A1 antibody disclosed herein comprises a Fc region with reduced or no Fc effector function. In some aspects, the constant regions comprise the amino acid sequences of the Fc region of a human IgG2 or IgG4, in some aspects, the anti-FAM19A1 antibody is of an IgG2/IgG4 isotype. In some aspects, the anti-FAM19A1 antibody comprises a chimeric Fc region which comprises a CH2 domain from an IgG antibody of the IgG4 isotype and a CH3 domain from an IgG antibody of the IgG1 isotype, or a chimeric Fc region which comprises a hinge region from IgG2 and a CH2 region from IgG4, or a Fc region with mutations that result in reduced or no Fc functions. Fc regions with reduced or no Fc effector function include those known in the art. See. e.g., Lau C. et al. J. Immunol. 191:4769-4777 (2013); An et al., mAbs 1:6, 572-579 (2009); and U.S. Pub. No. 20120100140 and the U.S. patents and publications and PCT publications cited therein. Also Fc regions with reduced or no Fc effector function can be readily made by a person of ordinary skill in the art

An anti-FAM19A1 antibody described herein that can be used for diagnostic purposes, including sample testing and in vivo imaging, and for this purpose, the antibody can be conjugated to an appropriate detectable agent, to form an immunoconjugate. For diagnostic purposes, appropriate agents are detectable labels that include radioisotopes, for whole body imaging, and radioisotopes, enzymes, fluorescent labels and other suitable antibody tags for sample testing.

The detectable labels can be any of the various types used in the field of in vitro diagnostics, including particulate labels including metal sols such as colloidal gold, isotopes such as I¹²⁵ or Tc⁹⁹ presented for instance with a peptidic chelating agent of the N2S2, N3S or N4 type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-STAR® or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

Immunoconjugates can be prepared by methods known in the art. Preferably, conjugation methods result in linkages which are substantially (or nearly) non-immunogenic, e.g., peptide- (i.e., amide-), sulfide-, (sterically hindered), disulfide-, hydrazone-, and ether linkages. These linkages are nearly non-immunogenic and show reasonable stability within serum (see, e.g., Senter, P. D., Curr. Opin. Chem. Biol. 13 (2009) 235-244; WO 2009/059278; WO 95/17886).

Depending on the biochemical nature of the moiety and the antibody, different conjugation strategies can be employed. In case the moiety is naturally occurring or recombinant of between 50 to 500 amino acids, there are standard procedures in text books describing the chemistry for synthesis of protein conjugates, which can be easily followed by the skilled artisan (see, e.g., Hackenberger, C. P. R., and Schwarzer, D., Angew. Chem. Int. Ed. Engl. 47 (2008) 10030-10074). In some aspects, the reaction of a maleinimido moiety with a cysteine residue within the antibody or the moiety is used. This is an especially suited coupling chemistry in case e.g. a Fab or Fab′-fragment of an antibody is used. Alternatively, in some aspects, coupling to the C-terminal end of the antibody or moiety is performed. C-terminal modification of a protein, e.g. of a Fab-fragment can e.g. be performed as described (Sunbul, M. and Yin, J., Org. Biomol. Chem. 7 (2009) 3361-3371).

In general, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see, e.g., Taki, M. et al., Prot. Eng. Des. Sel. 17 (2004) 119-126; Gautier, A. et al., Chem. Biol. 15 (2008) 128-136; and Protease-catalyzed formation of C—N bonds is used by Bordusa, F., Highlights in Bioorganic Chemistry (2004) 389-403).

Site specific reaction and covalent coupling can also be achieved by the selective reaction of terminal amino acids with appropriate modifying reagents. The reactivity of an N-terminal cysteine with benzonitrils (see Ren, H. et al., Angew. Chem. Int. Ed. Engl. 48 (2009) 9658-9662) can be used to achieve a site-specific covalent coupling. Native chemical ligation can also rely on C-terminal cysteine residues (Taylor, E. Vogel; Imperiali, B, Nucleic Acids and Molecular Biology (2009), 22 (Protein Engineering), 65-96).

EP 1 074 563 describes a conjugation method which is based on the faster reaction of a cysteine within a stretch of negatively charged amino acids with a cysteine located in a stretch of positively charged amino acids.

The moiety can also be a synthetic peptide or peptide mimic. In case a polypeptide is chemically synthesized, amino acids with orthogonal chemical reactivity can be incorporated during such synthesis (see, e.g., de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In order to obtain a mono-labeled polypeptide, the conjugate with 1:1 stoichiometry can be separated by chromatography from other conjugation side-products. This procedure can be facilitated by using a dye labeled binding pair member and a charged linker. By using this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

### V. Nucleic Acid Molecules

Another aspect described herein pertains to one or more nucleic acid molecules that encode any one of the antibodies, or antigen-binding fragments thereof, described herein. The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See F. Ausubel et al., ed. Current Protocols in Molecular Biology, Greene Publishing and Wiley Interscience, New York (1987). A nucleic acid described herein can be, for example, DNA or RNA and can or cannot contain intronic sequences. In certain aspects, the nucleic acid is a cDNA molecule.

Nucleic acids described herein can be obtained using standard molecular biology techniques. For antibodies expressed by hybridomas (e.g., hybridomas prepared from transgenic mice carrying human immunoglobulin genes as described further below), cDNAs encoding the light and heavy chains of the antibody made by the hybridoma can be obtained by standard PCR amplification or cDNA cloning techniques. For antibodies obtained from an immunoglobulin gene library (e.g., using phage display techniques), nucleic acid encoding the antibody can be recovered from the library.

Certain nucleic acids molecules described herein are those encoding the VH and VL sequences of the various anti-FAM19A1 antibodies of the present disclosure. Exemplary DNA sequences encoding the VH sequence of such antibodies are set forth in SEQ ID NOs: 38, 36, 40, and 42. Table 8. Exemplary DNA sequences encoding the VL sequences of such antibodies are set forth in SEQ ID NOs: 39, 37, 41, and 43. Table 9.

TABLE 8
Variable heavy chain polynucleotide sequence

| Antibody | VH Polynucleotide Sequence (SEQ ID NO) |
|---|---|
| Anti-FAM19A1 | CAGGTGCAGCTGGTGGAGTCGGGGGGAGGCGTGGCCCAGCCTGGGAGGTCCC |
| (“1C1”) | TGAGACTCTCCTGTGCAGCCTCTGGATTCGCCTTCAGTGACTATGGCATACA |
| | CTGGGTCCGCCAGGCTCCAGGCAAGGGGCTGGAGTGGGTGGCACTTATATCA |
| | TATGATGGAAGTAAGAGATCCTATGCAGACTCCGTGAAGGGCCGATTCGCCA |
| | TCTCCAGAGACAATTCCAAGAACACGCTGTATCTGCAAATGAACAGTCTGAG |
| | AGCCGAGGACACGGCCGTGTATTACTGTGCGAGACCGGGCGATTATGCCCTC |
| | GCTTTTGATCTCTGGGGCCAAGGGACAATGGTCACCGTCTCCTCA (SEQ ID |
| | NO: 38) |
| Anti-FAM19A1 | CAGGTGCAGCTGGTGGAGTCTGGGGCTGAGGTGAGGAAGCCTGGGGCCTCAG |
| (“1A11”) | TGAAGGTCTCCTGCAAGGCTTCTGGATATAGTTTCACCGGTTATGACATCAA |
| | CTGGGTGCGACAGGCCCCTGGACAAGGGCTTGAGTGGATGGGATGGATGAAC |
| | CCCAGTAGTGGTAATACAGGCTATGCACAGAAGTTTCAGGGCAGAGTCACCA |
| | TGACCAGGGACAGCTCCATAAGCACAGCCTACATGGAGCTGAGCAGCCTGAG |
| | ATCTGAGGACACGGCCGTGTATTACTGTGCGAGAGCTTTAAATTCGGTCTAC |
| | TACTACCACGCTCTGGACGTCTGGGGCCAAGGGACCACGATCACCGTCTCCT |
| | CA (SEQ ID NO: 36) |
| Anti-FAM19A1 | CAGGTGCAGCTGGTAGAGTCTGGGGGAGGCTTGGTAAAGCCAGGGCGGTCCC |
| (“2G7”) | TGAGCCTCTCCTGTACAACTTCTGGATTCCCCTTTGGTGATCATGCCATAAA |
| | TTGGGTCCGCCAGGCTCCAGGGAAGGGCCTGGAGTGGGTTGGTTTCATCAGG |
| | AGTAACACTTATGGTGGGACAACACAGTACGCCGCGTCTGTGGAGGGCAGAT |
| | TCACCATCTCAAGAGACGATTCCAAAAGCATCGCCTATCTGCAAATGAACAG |
| | TCTGAGAGCCGAGGACACGGCCGTGTATTACTGTACAAAAGATATAACCGGG |
| | GGCGGGTTTTGGAGCGACTACGGTGACTACTTTGATGCTTTTGATTTCTGGG |
| | GCCAAGGGACAATGGTCACCGTCTCCTCA (SEQ ID NO: 40) |
| Anti-FAM19A1 | CAGGTGCAGCTGCAGCAGTCGGGCCCAGGACTGGTGAAGCCTTCGGAGACCC |
| (“3A8”) | TGTCCCTCATCTGCAGTGTCTCTGGTGACGCCATCACCAGTGGTTCTTATTA |
| | CTGGGGCTGGATCCGCCAGTCCCCAGGGAGGGGGCTGGAGTGGATTGGGGAA |
| | ATCTCTCATAGTGGGAGCACCGACTACAACCCGTCCCTCAAGAGTCGAGTCA |
| | CCATATCAGTAGACAAGTCCAGGAACCAGTTCTCTCTGAGACTGAACTCTGT |
| | GACCGCCGTGGACACGGCCGTGTATTACTGTGCGGGAGATACAGCCTTGGTC |
| | GGTGCTTATTCTATCTGGGGCCAAGGGACAATGGTCACCGTCTCCTCA |
| | (SEQ ID NO: 42) |

TABLE 9
Variable light chain polynucleotide sequence

| Antibody | VL Polynucleotide Sequence (SEQ ID NO) |
|---|---|
| Anti-FAM19A1 | CAGACTGTGGTGACTCAGGAGCCATCGTTCTCAGTGTCCCCTGGAGAGACAG |
| (“1C1”) | TCACCCTCACTTGTGGCTTGAGCTCTGGCTCAGTCTCTACTTCTCACTTCCC |
| | CAGTTGGTACCGACAGACTCCAGGCCAGGCTCCACGCCCGCTCATCGACAGC |
| | AGAAACGCTCGCTCTATTGGGGTCCCTGATCGCTTCTCTGGCTCCATCGTTG |
| | GGACCAAGGCTACCCTGACCATCTCGGGGGCCCAGGCAGAAGATGAATGTAA |
| | TTATTACTGTGCTCTGTATATGGGTCGTGGCAATTGGGTGTTCGGCGGAGGG |
| | ACCAAGCTGACCGTCCTA (SEQ ID NO: 39) |
| Anti-FAM19A1 | TCCTATGAGCTGACACAGCCACCCTCAGCGTCCGTGTCCCCAGGACAGACAG |
| (“1A11”) | CCAGCATCACCTGCTCTGGAGATAGATTACACAATAAATATACTTCCTGGTA |
| | TCAACAGAAGCCAGGCCAGTCCCCTCTACTGGTCATCTATCAAGATGCCAAG |
| | CGACCCTCAGGGATCCCTGAGCGATTCTCGGGCTCCAGCTCTCGGGGCACAG |
| | CCACTCTGACCATCAGCGGGACCCAGGCTACGGATGAGGCTGACTATTACTG |
| | TCAAACGTGGGACAGGAGCACTGGAGTCTTCGGAACTGGGACCAAGGTCACC |
| | GTCCTA (SEQ ID NO: 37) |
| Anti-FAM19A1 | TCCTATGAGCTGACACAGCCACTCTCAGTGTCCGTGTCCCCAGGACAGACAG |
| (“2G7”) | CCAGCATCTCCTGCTCTGGGGATAAATTGGGTTACAAATATGCTTCCTGGTA |
| | TCAGCAGAAGCCGGGCCAGTCCCCTGTGGTGGTCATCTATCAAGATAAAAAG |
| | CGGCCCTCAGGGATCCCTGAGCGATTCTCTGGCTCCAACTCTGGGAACACAG |
| | CCACTCTGACCATCAGCGGGACCCAGCCTATGGATGAGGCTGACTATTATTG |
| | TCAGGCGTGGGACAGCGGCACTGCCTCTCATGTCTTCGGAACTGGGACCAAG |
| | GTCACCGTCCTA (SEQ ID NO: 41) |
| Anti-FAM19A1 | TCCTATGAGCTGACACAGGCACCCTCACTGTCCGTGTCGCCAGGACAGACAG |
| (“3A8”) | CCAACATCATCTGCTCTGGAGATAACTTGCGTACTAAATATGTTTCTTGGTA |
| | TCAGCAGAAGCCAGGCCAGTCCCCTTTATTGGTCATCTATCAGGACACCAGG |
| | CGGCCCTCAGGCATCCCTGCGCGATTCTCAGGCTCCAACTCGGGGAACACAG |
| | CCACTCTGACCATCAGCGGGACCCAGACTAGAGATGAATCTACCTATTACTG |
| | TATGACGTGGGACGTCGACACTACCTCGATGATTTTCGGCGGAGGGACCAAG |
| | CTGACCGTCCTA (SEQ ID NO: 43) |

A method for making an anti-FAM19A1 antibody (e.g., disclosed herein) can comprise expressing the relevant heavy chain and light chain of the antibody in a cell line comprising the nucleotide sequences encoding the heavy and light chains with a signal peptide. Host cells comprising these nucleotide sequences are encompassed herein.

Once DNA fragments encoding VH and VL segments are obtained, these DNA fragments can be further manipulated by standard recombinant DNA techniques, for example to convert the variable region genes to full-length antibody chain genes, to Fab fragment genes or to a scFv gene. In these manipulations, a VL- or VH-encoding DNA fragment is operatively linked to another DNA fragment encoding another protein, such as an antibody constant region or a flexible linker. The term “operatively linked”, as used in this context, is intended to mean that the two DNA fragments are joined such that the amino acid sequences encoded by the two DNA fragments remain in-frame.

The isolated DNA encoding the VH region can be converted to a full-length heavy chain gene by operatively linking the VH-encoding DNA to another DNA molecule encoding heavy chain constant regions (hinge, CH1, CH2 and/or CH3). The sequences of human heavy chain constant region genes are known in the art (see, e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The heavy chain constant region can be an IgG1, IgG2, IgG3, IgG4, IgA, IgE, IgM or IgD constant region, for example, an IgG2 and/or IgG 4 constant region. For a Fab fragment heavy chain gene, the VH-encoding DNA can be operatively linked to another DNA molecule encoding only the heavy chain CH1 constant region.

Isolated DNA encoding the VL region can be converted to a full-length light chain gene (as well as a Fab light chain gene) by operatively linking the VL-encoding DNA to another DNA molecule encoding the light chain constant region, CL. Sequences of human light chain constant region genes are known in the art (see, e.g., Kabat, E. A., et al., (1991) Sequences of Proteins of Immunological Interest, Fifth Edition, U.S. Department of Health and Human Services, NIH Publication No. 91-3242) and DNA fragments encompassing these regions can be obtained by standard PCR amplification. The light chain constant region can be a kappa or lambda constant region.

To create a scFv gene, the VH- and VL-encoding DNA fragments are operatively linked to another fragment encoding a flexible linker, e.g., encoding the amino acid sequence (Gly4-Ser)3, such that the VH and VL sequences can be expressed as a contiguous single-chain protein, with the VL and VH regions joined by the flexible linker (see, e.g., Bird et al., Science 242:423-426 (1988); Huston et al., Proc. Natl. Acad. Sci. USA 85:5879-5883 (1988); McCafferty et al., Nature 348:552-554 (1990)).

In some aspects, the vector disclosed herein comprises an isolated nucleic acid molecule comprising a nucleotide sequence encoding an antibody, or antigen-binding fragment thereof.

Suitable vectors for the disclosure include expression vectors, viral vectors, and plasmid vectors. In some aspects, the vector is a viral vector.

As used herein, an “expression vector” refers to any nucleic acid construct which contains the necessary elements for the transcription and translation of an inserted coding sequence, or in the case of an RNA viral vector, the necessary elements for replication and translation, when introduced into an appropriate host cell. Expression vectors can include plasmids, phagemids, viruses, and derivatives thereof.

### VI. Antibody Production

Anti-FAM19A1 antibodies disclosed herein can be produced by any method known in the art for the synthesis of antibodies, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See. e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, N.Y.; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In some aspects, an antibody described herein is an antibody (e.g., recombinant antibody) prepared, expressed, created, or isolated by any means that involves creation, e.g., via synthesis, genetic engineering of DNA sequences. In certain aspects, such antibody comprises sequences (e.g., DNA sequences or amino acid sequences) that do not naturally exist within the antibody germline repertoire of an animal or mammal (e.g., human) in vivo.

In a certain aspect, provided herein is a method of making an antibody or an antigen-binding fragment thereof which immunospecifically binds to FAM19A1 (e.g., human FAM19A1) comprising culturing a cell or host cell described herein. In a certain aspect, provided herein is a method of making an antibody or an antigen-binding fragment thereof which immunospecifically binds to FAM19A1 (e.g., human FAM19A1) comprising expressing (e.g., recombinantly expressing) the antibody or antigen-binding fragment thereof using a cell or host cell described herein (e.g., a cell or a host cell comprising polynucleotides encoding an antibody described herein). In some aspects, the cell is an isolated cell. In some aspects, the exogenous polynucleotides have been introduced into the cell. In certain aspects, the method further comprises the step of purifying the antibody or antigen-binding fragment thereof obtained from the cell or host cell.

Methods for producing polyclonal antibodies are known in the art (see, for example, Chapter 11 in: Short Protocols in Molecular Biology, (2002) 5th Ed., Ausubel F M et al., eds., John Wiley and Sons, New York).

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981). The term “monoclonal antibody” is not limited to antibodies produced through hybridoma technology. For example, monoclonal antibodies can be produced recombinantly from host cells exogenously expressing an antibody described herein or a fragment thereof, for example, light chain and/or heavy chain of such antibody.

In some aspects, a “monoclonal antibody,” as used herein, is an antibody produced by a single cell (e.g., hybridoma or host cell producing a recombinant antibody), wherein the antibody immunospecifically binds to FAM19A1 (e.g., human FAM19A1) as determined, e.g., by ELISA or other antigen-binding or competitive binding assay known in the art or in the Examples provided herein. In certain aspects, a monoclonal antibody can be a chimeric antibody or a humanized antibody. In certain aspects, a monoclonal antibody is a monovalent antibody or multivalent (e.g., bivalent) antibody. In further aspects, a monoclonal antibody is a monospecific or multispecific antibody (e.g., bispecific antibody). Monoclonal antibodies described herein can, for example, be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies expressed thereby are well known in the art (see, for example, Chapter 11 in: Short Protocols in Molecular Biology, (2002) 5th Ed., Ausubel F M et al., supra).

Methods for producing and screening for specific antibodies using hybridoma technology are routine and well known in the art. For example, in the hybridoma method, a mouse or other appropriate host animal, such as a sheep, goat, rabbit, rat, hamster or macaque monkey, is immunized to elicit lymphocytes that produce or are capable of producing antibodies that will specifically bind to the protein (e.g., human FAM19A1) used for immunization. Alternatively, lymphocytes can be immunized in vitro. Lymphocytes then are fused with myeloma cells using a suitable fusing agent, such as polyethylene glycol, to form a hybridoma cell (Goding J W (Ed), Monoclonal Antibodies: Principles and Practice, pp. 59-103 (Academic Press, 1986)). Additionally, a RIMMS (repetitive immunization multiple sites) technique can be used to immunize an animal (Kilpatrick K E et al., (1997) Hybridoma 16:381-9, incorporated by reference in its entirety).

In some aspects, mice (or other animals, such as chickens, rats, monkeys, donkeys, pigs, sheep, hamster, or dogs) can be immunized with an antigen (e.g., FAM19A1 such as human FAM19A1) and once an immune response is detected, e.g., antibodies specific for the antigen are detected in the mouse serum, the mouse spleen is harvested and splenocytes isolated. The splenocytes are then fused by well-known techniques to any suitable myeloma cells, for example cells from cell line SP20 available from the American Type Culture Collection (ATCC) (Manassas, Va.), to form hybridomas. Hybridomas are selected and cloned by limited dilution. In certain aspects, lymph nodes of the immunized mice are harvested and fused with NSO myeloma cells.

The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Specific aspects employ myeloma cells that fuse efficiently, support stable high-level production of antibody by the selected antibody-producing cells, and are sensitive to a medium such as HAT medium. Among these myeloma cell lines are murine myeloma lines, such as NSO cell line or those derived from MOPC-21 and MPC-11 mouse tumors available from the Salk Institute Cell Distribution Center, San Diego, Calif., USA, and SP-2 or X63-Ag8.653 cells available from the American Type Culture Collection, Rockville, Md., USA. Human myeloma and mouse-human heteromyeloma cell lines also have been described for the production of human monoclonal antibodies (Kozbor D (1984) J Immunol 133: 3001-5; Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against FAM19A1 (e.g., human FAM19A1). The binding specificity of monoclonal antibodies produced by hybridoma cells is determined by methods known in the art, for example, immunoprecipitation or by an in vitro binding assay, such as radioimmunoassay (RIA) or enzyme-linked immunoabsorbent assay (ELISA).

After hybridoma cells are identified that produce antibodies of the desired specificity, affinity, and/or activity, the clones can be subcloned by limiting dilution procedures and grown by standard methods (Goding J W (Ed), Monoclonal Antibodies: Principles and Practice, supra). Suitable culture media for this purpose include, for example, D-MEM or RPMI 1640 medium. In addition, the hybridoma cells can be grown in vivo as ascites tumors in an animal.

The monoclonal antibodies secreted by the subclones are suitably separated from the culture medium, ascites fluid, or serum by conventional immunoglobulin purification procedures such as, for example, protein A-Sepharose, hydroxylapatite chromatography, gel electrophoresis, dialysis, or affinity chromatography.

Antibodies described herein include antibody fragments which recognize specific FAM19A1 (e.g., human FAM19A1) and can be generated by any technique known to those of skill in the art. For example, Fab and F(ab′)2 fragments described herein can be produced by proteolytic cleavage of immunoglobulin molecules, using enzymes such as papain (to produce Fab fragments) or pepsin (to produce F(ab′)2 fragments). A Fab fragment corresponds to one of the two identical arms of an antibody molecule and contains the complete light chain paired with the VH and CH1 domains of the heavy chain. A F(ab′)2 fragment contains the two antigen-binding arms of an antibody molecule linked by disulfide bonds in the hinge region.

Further, the antibodies described herein or antigen-binding fragments thereof can also be generated using various phage display methods known in the art. In phage display methods, functional antibody domains are displayed on the surface of phage particles which carry the polynucleotide sequences encoding them. In particular, DNA sequences encoding VH and VL domains are amplified from animal cDNA libraries (e.g., human or non-human such as murine or chicken cDNA libraries of affected tissues). The DNAs encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antigen-binding domain that binds to a particular antigen can be selected or identified with antigen, e.g., using labeled antigen or antigen bound or captured to a solid surface or bead. Examples of phage display methods that can be used to make the antibodies described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce antibody fragments such as Fab, Fab′ and F(ab′)2 fragments can also be employed using methods known in the art such as those disclosed in PCT publication No. WO 92/22324; Mullinax R L et al., (1992) BioTechniques 12(6): 864-9; Sawai H et al., (1995) Am J Reprod Immunol 34: 26-34; and Better M et al., (1988) Science 240: 1041-1043.

In one aspect, to generate whole antibodies, PCR primers including VH or VL nucleotide sequences, a restriction site, and a flanking sequence to protect the restriction site can be used to amplify the VH or VL sequences from a template, e.g., scFv clones. Utilizing cloning techniques known to those of skill in the art, the PCR amplified VH domains can be cloned into vectors expressing a VH constant region, and the PCR amplified VL domains can be cloned into vectors expressing a VL constant region, e.g., human kappa or lambda constant regions. The VH and VL domains can also be cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

A chimeric antibody is a molecule in which different portions of the antibody are derived from different immunoglobulin molecules. For example, a chimeric antibody can contain a variable region of a non-human animal (e.g., mouse, rat or chicken) monoclonal antibody fused to a constant region of a human antibody. Methods for producing chimeric antibodies are known in the art. See. e.g., Morrison S L (1985) Science 229: 1202-7; Oi V T & Morrison S L (1986) BioTechniques 4: 214-221; Gillies S D et al., (1989) J Immunol Methods 125: 191-202; and U.S. Pat. Nos. 5,807,715, 4,816,567, 4,816,397, and 6,331,415.

A humanized antibody is capable of binding to a predetermined antigen and which comprises a framework region having substantially the amino acid sequence of a human immunoglobulin and CDRs having substantially the amino acid sequence of a non-human immunoglobulin (e.g., a murine or a chicken immunoglobulin). In particular aspects, a humanized antibody also comprises at least a portion of an immunoglobulin constant region (Fc), typically that of a human immunoglobulin. The antibody also can include the CH1, hinge, CH2, CH3, and CH4 regions of the heavy chain. A humanized antibody can be selected from any class of immunoglobulins, including IgM, IgG, IgD, IgA, and IgE, and any isotype, including IgG1, IgG2, IgG3, and IgG4. Humanized antibodies can be produced using a variety of techniques known in the art, including but not limited to, CDR-grafting (European Patent No. EP 239400; International Publication No. WO 91/09967; and U.S. Pat. Nos. 5,225,539, 5,530,101, and 5,585,089), veneering or resurfacing (European Patent Nos. EP 592106 and EP 519596; Padlan E A (1991) Mol Immunol 28(4/5): 489-498; Studnicka G M et al., (1994) Prot Engineering 7(6): 805-814; and Roguska M A et al., (1994) PNAS 91: 969-973), chain shuffling (U.S. Pat. No. 5,565,332), and techniques disclosed in, e.g., U.S. Pat. Nos. 6,407,213, 5,766,886, International Publication No. WO 93/17105; Tan P et al., (2002) J Immunol 169: 1119-25; Caldas C et al., (2000) Protein Eng. 13(5): 353-60; Morea V et al., (2000) Methods 20(3): 267-79; Baca M et al., (1997) J Biol Chem 272(16): 10678-84; Roguska M A et al., (1996) Protein Eng 9(10): 895 904; Couto J R et al., (1995) Cancer Res. 55 (23 Supp): 5973s-5977s; Couto J R et al., (1995) Cancer Res 55(8): 1717-22; Sandhu J S (1994) Gene 150(2): 409-10 and Pedersen J T et al., (1994) J Mol Biol 235(3): 959-73. See also U.S. Application Publication No. US 2005/0042664 A1 (Feb. 24, 2005), which is incorporated by reference herein in its entirety.

Methods for making multispecific (e.g., bispecific antibodies) have been described. See, for example, U.S. Pat. Nos. 7,951,917; 7,183,076; 8,227,577; 5,837,242; 5,989,830; 5,869,620; 6,132,992 and 8,586,713.

Single domain antibodies, for example, antibodies lacking the light chains, can be produced by methods well known in the art. See Riechmann L & Muyldermans S (1999) J Immunol 231: 25-38; Nuttall S D et al., (2000) Curr Pharm Biotechnol 1(3): 253-263; Muyldermans S, (2001) J Biotechnol 74(4): 277-302; U.S. Pat. No. 6,005,079; and International Publication Nos. WO 94/04678, WO 94/25591 and WO 01/44301.

Further, antibodies that immunospecifically bind to a FAM19A1 antigen can, in turn, be utilized to generate anti-idiotype antibodies that “mimic” an antigen using techniques well known to those skilled in the art. (See. e.g., Greenspan N S & Bona C A (1989) FASEB J 7(5): 437-444; and Nissinoff A (1991) J Immunol 147(8): 2429-2438).

In particular aspects, an antibody described herein, which binds to the same epitope of FAM19A1 (e.g., human FAM19A1) as an anti-FAM19A1 antibody described herein, is a human antibody or an antigen-binding fragment thereof. In particular aspects, an antibody described herein, which competitively blocks (e.g., in a dose-dependent manner) antibodies described herein, (e.g., 1C1, 1A11, 2G7, and 3A8) from binding to FAM19A1 (e.g., human FAM19A1), is a human antibody or an antigen-binding fragment thereof.

Human antibodies can be produced using any method known in the art. For example, transgenic mice which are incapable of expressing functional endogenous immunoglobulins, but which can express human immunoglobulin genes, can be used. In particular, the human heavy and light chain immunoglobulin gene complexes can be introduced randomly or by homologous recombination into mouse embryonic stem cells. Alternatively, the human variable region, constant region, and diversity region can be introduced into mouse embryonic stem cells in addition to the human heavy and light chain genes. The mouse heavy and light chain immunoglobulin genes can be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then bred to produce homozygous offspring which express human antibodies. The transgenic mice are immunized in the normal fashion with a selected antigen, e.g., all or a portion of an antigen (e.g., FAM19A1). Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg N & Huszar D (1995) Int Rev Immunol 13:65-93. For a detailed discussion of this technology for producing human antibodies and human monoclonal antibodies and protocols for producing such antibodies, see. e.g., International Publication Nos. WO 98/24893, WO 96/34096 and WO 96/33735; and U.S. Pat. Nos. 5,413,923, 5,625,126, 5,633,425, 5,569,825, 5,661,016, 5,545,806, 5,814,318 and 5,939,598. Examples of mice capable of producing human antibodies include the XENOMOUSE™ (Abgenix, Inc.; U.S. Pat. Nos. 6,075,181 and 6,150,184), the HUAB-MOUSE™ (Mederex, Inc./Gen Pharm; U.S. Pat. Nos. 5,545,806 and 5,569,825), the TRANS CHROMO MOUSE™ (Kirin) and the KM MOUSE™ (Medarex/Kirin).

Human antibodies which specifically bind to FAM19A1 (e.g., human FAM19A1) can be made by a variety of methods known in the art including phage display methods described above using antibody libraries derived from human immunoglobulin sequences. See also U.S. Pat. Nos. 4,444,887, 4,716,111, and 5,885,793; and International Publication Nos. WO 98/46645, WO 98/50433, WO 98/24893, WO 98/16654, WO 96/34096, WO 96/33735, and WO 91/10741.

In some aspects, human antibodies can be produced using mouse-human hybridomas. For example, human peripheral blood lymphocytes transformed with Epstein-Barr virus (EBV) can be fused with mouse myeloma cells to produce mouse-human hybridomas secreting human monoclonal antibodies, and these mouse-human hybridomas can be screened to determine ones which secrete human monoclonal antibodies that immunospecifically bind to a target antigen (e.g., FAM19A1 such as human FAM19A1)). Such methods are known and are described in the art, see, e.g., Shinmoto H et al., (2004) Cytotechnology 46: 19-23; Naganawa Y et al., (2005) Human Antibodies 14: 27-31.

### VII. Methods of Engineering Antibodies

As discussed above, the anti-FAM19A1 antibody or antigen-binding portion thereof having VH and VL sequences disclosed herein can be used to create new anti-FAM19A1 antibody or antigen-binding portion thereof by modifying the VH and/or VL sequences, or the constant region(s) attached thereto. Thus, in another aspect described herein, the structural features of an anti-FAM19A1 antibody described herein, e.g., 1C1, 1A11, 2G7, and 3A8, is used to create structurally related anti-FAM19A1 antibodies that retain at least one functional property of the antibodies described herein, such as binding to human FAM19A1. For example, the starting material for the engineering method is VH and/or VL sequences provided herein, or one or more CDR regions thereof. To create the engineered antibody, it is not necessary to actually prepare (i.e., express as a protein) an antibody having one or more of the VH and/or VL sequences provided herein, or one or more CDR regions thereof. Rather, the information contained in the sequence(s) is used as the starting material to create a “second generation” sequence(s) derived from the original sequence(s) and then the “second generation” sequence(s) is prepared and expressed as a protein.

Accordingly, provided herein are methods for preparing an anti-FAM19A1 antibody or antigen-binding portion thereof comprising:
(a) providing: (i) a heavy chain variable region sequence comprising a CDR1, CDR2, and/or CDR3 sequence as set forth in Table 4 or a CDR1, CDR2, and/or CDR3 of the heavy chain variable region as set forth in Table 6; and (ii) a light chain variable region sequence comprising a CDR1, CDR2, and/or CDR3 sequence as set forth in Table 5 or a CDR1, CDR2, and/or CDR3 of the light chain variable region as set forth in Table 7;
(b) altering at least one amino acid residue within the heavy chain variable region sequence and/or the light chain variable region sequence to create at least one altered antibody or antigen-binding portion sequence; and
(c) expressing the altered antibody or antigen-binding portion sequence as a protein.

Standard molecular biology techniques can be used to prepare and express the altered antibody or antigen-binding portion sequence.

In some aspects, the antibody or antigen-binding portion thereof encoded by the altered antibody or antigen-binding portion sequence(s) is one that retains one, some or all of the functional properties of the anti-FAM19A1 antibodies described herein. Non-limiting examples of such properties include,
(1) binds to soluble human FAM19A1 with a K_{D} of 10 nM or less, e.g., as measured by BIACORE™ or ELISA;
(2) binds to membrane bound human FAM19A1 with a K_{D} of 10 nM or less, e.g., as measured by BIACORE™ or ELISA;
(3) promotes the differentiation of neurons;
(4) increases neurite outgrowth in differentiated neurons;
(5) reduces, reverses, and/or prevents one or more symptoms associated with glaucoma;
(6) improves retinal potential (e.g., as evidenced by increased oscillatory potential);
(7) reduces and/or restores the loss of retinal ganglion cells (e.g., observed in glaucoma subjects);
(8) reduces, reverses, and/or prevents one or more symptoms associated with neuropathic pain;
(9) increases latency and/or threshold to an external stimulus; and
(10) increases and/or regulates sensory nerve conduction velocity.

In certain aspects of the methods of engineering antibodies described herein, mutations can be introduced randomly or selectively along all or part of an anti-FAM19A1 antibody coding sequence and the resulting modified anti-FAM19A1 antibodies can be screened for binding activity and/or other functional properties as described herein. Mutational methods have been described in the art. For example, PCT Publication WO 02/092780 by Short describes methods for creating and screening antibody mutations using saturation mutagenesis, synthetic ligation assembly, or a combination thereof. Alternatively, PCT Publication WO 03/074679 by Lazar et al. describes methods of using computational screening methods to optimize physiochemical properties of antibodies.

### VIII. Cells and Vectors

In certain aspects, provided herein are cells (e.g., host cells) expressing (e.g., recombinantly) antibodies described herein (or an antigen-binding fragment thereof) which specifically bind to FAM19A1 (e.g., human FAM19A1) and related polynucleotides and expression vectors. Provided herein are vectors (e.g., expression vectors) comprising polynucleotides comprising nucleotide sequences encoding anti-FAM19A1 antibodies or a fragment for recombinant expression in host cells, e.g., in mammalian cells. Also provided herein are host cells comprising such vectors for recombinantly expressing anti-FAM19A1 antibodies described herein (e.g., human or humanized antibody). In a particular aspect, provided herein are methods for producing an antibody described herein, comprising expressing such antibody from a host cell.

Recombinant expression of an antibody described herein (e.g., a full-length antibody, heavy and/or light chain of an antibody, or a single chain antibody described herein) that specifically binds to FAM19A1 (e.g., human FAM19A1) involves construction of an expression vector containing a polynucleotide that encodes the antibody. Once a polynucleotide encoding an antibody molecule, heavy and/or light chain of an antibody, or a fragment thereof (e.g., heavy and/or light chain variable domains) described herein has been obtained, the vector for the production of the antibody molecule can be produced by recombinant DNA technology using techniques well known in the art. Thus, methods for preparing a protein by expressing a polynucleotide containing an antibody or antibody fragment (e.g., light chain or heavy chain) encoding nucleotide sequence are described herein. Methods which are well known to those skilled in the art can be used to construct expression vectors containing antibody or antibody fragment (e.g., light chain or heavy chain) coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding an antibody molecule described herein, a heavy or light chain of an antibody, a heavy or light chain variable domain of an antibody or a fragment thereof, or a heavy or light chain CDR, operably linked to a promoter. Such vectors can, for example, include the nucleotide sequence encoding the constant region of the antibody molecule (see. e.g., International Publication Nos. WO 86/05807 and WO 89/01036; and U.S. Pat. No. 5,122,464, each of which are herein incorporated by reference in its entirety) and variable domains of the antibody can be cloned into such a vector for expression of the entire heavy, the entire light chain, or both the entire heavy and light chains.

An expression vector can be transferred to a cell (e.g., host cell) by conventional techniques and the resulting cells can then be cultured by conventional techniques to produce an antibody described herein (e.g., an antibody comprising the VH and/or VL, or one or more of the VH and/or VL CDRs, of an anti-FAM19A1 antibody of the present disclosure) or a fragment thereof. Thus, provided herein are host cells containing a polynucleotide encoding an antibody described herein or fragments thereof, or a heavy or light chain thereof, or fragment thereof, or a single chain antibody described herein, operably linked to a promoter for expression of such sequences in the host cell. In certain aspects, for the expression of double-chained antibodies, vectors encoding both the heavy and light chains, individually, can be co-expressed in the host cell for expression of the entire immunoglobulin molecule, as detailed below. In certain aspects, a host cell contains a vector comprising a polynucleotide encoding both the heavy chain and light chain of an antibody described herein, or a fragment thereof. In specific aspects, a host cell contains two different vectors, a first vector comprising a polynucleotide encoding a heavy chain or a heavy chain variable region of an antibody described herein, or a fragment thereof, and a second vector comprising a polynucleotide encoding a light chain or a light chain variable region of an antibody described herein, or a fragment thereof. In some aspects, a first host cell comprises a first vector comprising a polynucleotide encoding a heavy chain or a heavy chain variable region of an antibody described herein, or a fragment thereof, and a second host cell comprises a second vector comprising a polynucleotide encoding a light chain or a light chain variable region of an antibody described herein. In specific aspects, a heavy chain/heavy chain variable region expressed by a first cell associated with a light chain/light chain variable region of a second cell to form an anti-FAM19A1 antibody described herein or an antigen-binding fragment thereof. In certain aspects, provided herein is a population of host cells comprising such first host cell and such second host cell.

In some aspects, provided herein is a population of vectors comprising a first vector comprising a polynucleotide encoding a light chain/light chain variable region of an anti-FAM19A1 antibody described herein, and a second vector comprising a polynucleotide encoding a heavy chain/heavy chain variable region of an anti-FAM19A1 antibody described herein.

A variety of host-expression vector systems can be utilized to express antibody molecules described herein. Such host-expression systems represent vehicles by which the coding sequences of interest can be produced and subsequently purified, but also represent cells which can, when transformed or transfected with the appropriate nucleotide coding sequences, express an antibody molecule described herein in situ. These include but are not limited to microorganisms such as bacteria (e.g., E. coli and B. subtilis) transformed with recombinant bacteriophage DNA, plasmid DNA or cosmid DNA expression vectors containing antibody coding sequences; yeast (e.g., Saccharomyces Pichia) transformed with recombinant yeast expression vectors containing antibody coding sequences; insect cell systems infected with recombinant virus expression vectors (e.g., baculovirus) containing antibody coding sequences; plant cell systems (e.g., green algae such as Chlamydomonas reinhardtii) infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS (e.g., COS1 or COS), CHO, BHK, MDCK, HEK 293, NSO, PER.C6, VERO, CRL7030, HsS78Bst, HeLa, and NIH 3T3, HEK-293T, HepG2, SP210, Rl .l, B-W, L-M, BSCl, BSC40, YB/20 and BMT10 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). In a specific aspect, cells for expressing antibodies described herein or an antigen-binding fragment thereof are CHO cells, for example CHO cells from the CHO GS SYSTEM™ (Lonza). In some aspects, cells for expressing antibodies described herein are human cells, e.g., human cell lines. In some aspects, a mammalian expression vector is POPTIVEC™ or pcDNA3.3. In some aspects, bacterial cells such as Escherichia coli, or eukaryotic cells (e.g., mammalian cells), especially for the expression of whole recombinant antibody molecule, are used for the expression of a recombinant antibody molecule. For example, mammalian cells such as Chinese hamster ovary (CHO) cells, in conjunction with a vector such as the major intermediate early gene promoter element from human cytomegalovirus is an effective expression system for antibodies (Foecking M K & Hofstetter H (1986) Gene 45: 101-5; and Cockett M I et al., (1990) Biotechnology 8(7): 662-7). In certain aspects, antibodies described herein are produced by CHO cells or NSO cells. In some aspects, the expression of nucleotide sequences encoding antibodies described herein which immunospecifically bind FAM19A1 (e.g., human FAM19A1) is regulated by a constitutive promoter, inducible promoter or tissue specific promoter.

In bacterial systems, a number of expression vectors can be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B (1983) EMBO J 2: 1791-1794), in which the antibody coding sequence can be ligated individually into the vector in frame with the lac Z coding region so that a fusion protein is produced; pIN vectors (Inouye S & Inouye M (1985) Nuc Acids Res 13: 3101-3109; Van Heeke G & Schuster S M (1989) J Biol Chem 24: 5503-5509); and the like. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The antibody coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the antibody coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (see. e.g., Logan J & Shenk T (1984) PNAS 81(12): 3655-9). Specific initiation signals can also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153: 516-544).

In addition, a host cell strain can be chosen which modulates the expression of the inserted sequences, or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, MDCK, HEK 293, NIH 3T3, W138, BT483, Hs578T, HTB2, BT20 and T47D, NSO (a murine myeloma cell line that does not endogenously produce any immunoglobulin chains), CRL7030, COS (e.g., COS 1 or COS), PER.C6, VERO, HsS78Bst, HEK-293T, HepG2, SP210, R1.1, B-W, L-M, BSC 1, BSC40, YB/20, BMT10 and HsS78Bst cells. In certain aspects, anti-FAM19A1 antibodies described herein are produced in mammalian cells, such as CHO cells.

In some aspects, the antibodies described herein or antigen-binding portions thereof have reduced fucose content or no fucose content. Such antibodies can be produced using techniques known one skilled in the art. For example, the antibodies can be expressed in cells deficient or lacking the ability of to fucosylate. In a specific example, cell lines with a knockout of both alleles of 1,6-fucosyltransferase can be used to produce antibodies or antigen-binding portions thereof with reduced fucose content. The POTELLIGENT® system (Lonza) is an example of such a system that can be used to produce antibodies or antigen-binding portions thereof with reduced fucose content.

For long-term, high-yield production of recombinant proteins, stable expression cells can be generated. For example, cell lines which stably express an anti-FAM19A1 antibody described herein an antigen-binding portion thereof can be engineered. In specific aspects, a cell provided herein stably expresses a light chain/light chain variable domain and a heavy chain/heavy chain variable domain which associate to form an antibody described herein or an antigen-binding portion thereof.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.) and a selectable marker. Following the introduction of the foreign DNA/polynucleotide, engineered cells can be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method can advantageously be used to engineer cell lines which express an anti-FAM19A1 antibody described herein or an antibody binding portion thereof. Such engineered cell lines can be particularly useful in screening and evaluation of compositions that interact directly or indirectly with the antibody molecule.

A number of selection systems can be used, including but not limited to, the herpes simplex virus thymidine kinase (Wigler M et al., (1977) Cell 11(1): 223-32), hypoxanthineguanine phosphoribosyltransferase (Szybalska E H & Szybalski W (1962) PNAS 48(12): 2026-2034) and adenine phosphoribosyltransferase (Lowy I et al., (1980) Cell 22(3): 817-23) genes can be employed in tk-, hgprt- or aprt-cells, respectively. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78: 1527-31); gpt, which confers resistance to mycophenolic acid (Mulligan R C & Berg P (1981) PNAS 78(4): 2072-6); neo, which confers resistance to the aminoglycoside G-418 (Wu G Y & Wu C H (1991) Biotherapy 3: 87-95; Tolstoshev P (1993) Ann Rev Pharmacol Toxicol 32: 573-596; Mulligan R C (1993) Science 260: 926-932; and Morgan R A & Anderson W F (1993) Ann Rev Biochem 62: 191-217; Nabel G J & Feigner P L (1993) Trends Biotechnol 11(5): 211-5); and hygro, which confers resistance to hygromycin (Santerre R F et al., (1984) Gene 30(1-3): 147-56). Methods commonly known in the art of recombinant DNA technology can be routinely applied to select the desired recombinant clone and such methods are described, for example, in Ausubel F M et al., (eds.), Current Protocols in Molecular Biology, John Wiley & Sons, N Y (1993); Kriegler M, Gene Transfer and Expression, A Laboratory Manual, Stockton Press, N Y (1990); and in Chapters 12 and 13, Dracopoli N C et al., (eds.), Current Protocols in Human Genetics, John Wiley & Sons, N Y (1994); Colbere-Garapin F et al., (1981) J Mol Biol 150: 1-14, which are incorporated by reference herein in their entireties.

The expression levels of an antibody molecule can be increased by vector amplification (for a review, see Bebbington C R & Hentschel C C G, The use of vectors based on gene amplification for the expression of cloned genes in mammalian cells in DNA cloning, Vol. 3 (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse G F et al., (1983)Mol Cell Biol 3: 257-66).

The host cell can be co-transfected with two or more expression vectors described herein, the first vector encoding a heavy chain derived polypeptide and the second vector encoding a light chain derived polypeptide. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both heavy and light chain polypeptides. In such situations, the light chain should be placed before the heavy chain to avoid an excess of toxic free heavy chain (Proudfoot N J (1986) Nature 322: 562-565; and Kohler G (1980) PNAS 77: 2197-2199). The coding sequences for the heavy and light chains can comprise cDNA or genomic DNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., heavy chain of an antibody described herein), and a second gene and (e.g., light chain of an antibody described herein). In such vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once an antibody molecule described herein has been produced by recombinant expression, it can be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies described herein can be fused to heterologous polypeptide sequences described herein or otherwise known in the art to facilitate purification.

In specific aspects, an antibody or an antigen-binding portion thereof described herein is isolated or purified. Generally, an isolated antibody is one that is substantially free of other antibodies with different antigenic specificities than the isolated antibody. For example, in some aspects, a preparation of an antibody described herein is substantially free of cellular material and/or chemical precursors. The language “substantially free of cellular material” includes preparations of an antibody in which the antibody is separated from cellular components of the cells from which it is isolated or recombinantly produced. Thus, an antibody that is substantially free of cellular material includes preparations of antibody having less than about 30%, 20%, 10%, 5%, 2%, 1%, 0.5%, or 0.1% (by dry weight) of heterologous protein (also referred to herein as a “contaminating protein”) and/or variants of an antibody, for example, different post-translational modified forms of an antibody or other different versions of an antibody (or antibody binding portions). When the antibody is recombinantly produced, it is also generally substantially free of culture medium, i.e., culture medium represents less than about 20%>, 10%>, 2%, 1%, 0.5%, or 0.1% of the volume of the protein preparation. When the antibody is produced by chemical synthesis, it is generally substantially free of chemical precursors or other chemicals, i.e., it is separated from chemical precursors or other chemicals which are involved in the synthesis of the protein. Accordingly, such preparations of the antibody have less than about 30%, 20%, 10%, or 5% (by dry weight) of chemical precursors or compounds other than the antibody of interest. In some aspects, antibodies described herein are isolated or purified.

### IX. Diagnosis

As described supra, FAM19A1 antagonists described herein (e.g., anti-FAM19A1 antibody) can be used for diagnostic purposes, including sample testing and in vivo imaging, and for this purpose, the antibody (or binding portion thereof) can be conjugated to an appropriate detectable agent, to form an immunoconjugate. For diagnostic purposes, appropriate agents are detectable labels that include radioisotopes, for whole body imaging, and radioisotopes, enzymes, fluorescent labels and other suitable antibody tags for sample testing.

Detectable labels can be any of the various types used currently in the field of in vitro diagnostics, including particulate labels including metal sols such as colloidal gold, isotopes such as I¹²⁵ or Tc⁹⁹ presented for instance with a peptidic chelating agent of the N2S2, N3S or N4 type, chromophores including fluorescent markers, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-STAR or other luminescent substrates well-known to those in the art, e.g., the chelates of suitable lanthanides such as Terbium(III) and Europium(III). The detection means is determined by the chosen label. Appearance of the label or its reaction products can be achieved using the naked eye, in the case where the label is particulate and accumulates at appropriate levels, or using instruments such as a spectrophotometer, a luminometer, a fluorimeter, and the like, all in accordance with standard practice.

FAM19A1 antagonists described herein (e.g., anti-FAM19A1 antibody) can also be conjugated to a therapeutic agent to form an immunoconjugate such as an antibody-drug conjugate (ADC). Suitable therapeutic agents include agents that can treat an abnormality of CNS function or a disease and disorders associated with such abnormality (e.g., glaucoma or neuropathic pain). Non-limiting examples of such therapeutic agents are provided throughout the present disclosure.

Immunoconjugates can be prepared by methods known in the art. In some aspects, conjugation methods result in linkages which are substantially (or nearly) non-immunogenic, e.g., peptide- (i.e., amide-), sulfide-, (sterically hindered), disulfide-, hydrazone-, and ether linkages. These linkages are nearly non-immunogenic and show reasonable stability within serum (see. e.g., Senter, P. D., Curr. Opin. Chem. Biol. 13 (2009) 235-244; WO 2009/059278; WO 95/17886).

Depending on the biochemical nature of the moiety and the antibody, different conjugation strategies can be employed. In case the moiety is naturally-occurring or recombinant of between 50 to 500 amino acids, there are standard procedures in text books describing the chemistry for synthesis of protein conjugates, which can be easily followed by the skilled artisan (see, e.g., Hackenberger, C. P. R., and Schwarzer, D., Angew. Chem. Int. Ed. Engl. 47 (2008) 10030-10074). In some aspects, the reaction of a maleinimido moiety with a cysteine residue within the antibody or the moiety is used. This is an especially suited coupling chemistry in case e.g., a Fab or Fab′-fragment of an antibody is used. Alternatively, in some aspects, coupling to the C-terminal end of the antibody or moiety is performed. C-terminal modification of a protein, e.g. of a Fab-fragment can e.g. be performed as described (Sunbul, M. and Yin, J., Org. Biomol. Chem. 7 (2009) 3361-3371).

In general, site specific reaction and covalent coupling is based on transforming a natural amino acid into an amino acid with a reactivity which is orthogonal to the reactivity of the other functional groups present. For example, a specific cysteine within a rare sequence context can be enzymatically converted in an aldehyde (see Frese, M. A., and Dierks, T., ChemBioChem. 10 (2009) 425-427). It is also possible to obtain a desired amino acid modification by utilizing the specific enzymatic reactivity of certain enzymes with a natural amino acid in a given sequence context (see. e.g., Taki, M. et al., Prot. Eng. Des. Sel. 17 (2004) 119-126; Gautier, A. et al., Chem. Biol. 15 (2008) 128-136; and Protease-catalyzed formation of C— N bonds is used by Bordusa, F., Highlights in Bioorganic Chemistry (2004) 389-403).

Site specific reaction and covalent coupling can also be achieved by the selective reaction of terminal amino acids with appropriate modifying reagents. The reactivity of an N-terminal cysteine with benzonitrils (see Ren, H. et al., Angew. Chem. Int. Ed. Engl. 48 (2009) 9658-9662) can be used to achieve a site-specific covalent coupling. Native chemical ligation can also rely on C-terminal cysteine residues (Taylor, E. Vogel; Imperiali, B, Nucleic Acids and Molecular Biology (2009), 22 (Protein Engineering), 65-96).

EP 1 074 563 describes a conjugation method which is based on the faster reaction of a cysteine within a stretch of negatively charged amino acids with a cysteine located in a stretch of positively charged amino acids.

The moiety can also be a synthetic peptide or peptide mimic. In case a polypeptide is chemically synthesized, amino acids with orthogonal chemical reactivity can be incorporated during such synthesis (see, e.g., de Graaf, A. J. et al., Bioconjug. Chem. 20 (2009) 1281-1295). Since a great variety of orthogonal functional groups is at stake and can be introduced into a synthetic peptide, conjugation of such peptide to a linker is standard chemistry.

In order to obtain a mono-labeled polypeptide, the conjugate with 1:1 stoichiometry can be separated by chromatography from other conjugation side-products. This procedure can be facilitated by using a dye labeled binding pair member and a charged linker. By using this kind of labeled and highly negatively charged binding pair member, mono conjugated polypeptides are easily separated from non-labeled polypeptides and polypeptides which carry more than one linker, since the difference in charge and molecular weight can be used for separation. The fluorescent dye can be useful for purifying the complex from un-bound components, like a labeled monovalent binder.

### X. Pharmaceutical Compositions

Provided herein are compositions comprising a FAM19A1 antagonist disclosed herein (e.g., anti-FAM19A1 antibody) having the desired degree of purity in a physiologically acceptable carrier, excipient or stabilizer (Remington's Pharmaceutical Sciences (1990) Mack Publishing Co., Easton, Pa.). Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN®, PLURONICS® or polyethylene glycol (PEG).

In some aspects, pharmaceutical compositions comprise an antibody or antigen-binding fragment thereof, a bispecific molecule, or a immunoconjugate described herein, and optionally one or more additional prophylactic or therapeutic agents, in a pharmaceutically acceptable carrier. In certain aspects, pharmaceutical compositions comprise an effective amount of an antibody or antigen-binding fragment thereof described herein, and optionally one or more additional prophylactic of therapeutic agents, in a pharmaceutically acceptable carrier. In some aspects, the antibody is the only active ingredient included in the pharmaceutical composition. Pharmaceutical compositions described herein can be useful in reducing a FAM19A1 activity and thereby treat, e.g., a disease or disorder associated with an abnormality in CNS function (e.g., glaucoma and neuropathic pain).

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances. Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations can be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

A pharmaceutical composition can be formulated for any route of administration. Specific examples include intranasal, oral, parenterally, intrathecally, intra-cerebroventricularly, pulmonarily, subcutaneously, or intraventricularly. Parenteral administration, characterized by either subcutaneous, intramuscular or intravenous injection, is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered can also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Preparations for parenteral administration of an antibody include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Topical mixtures comprising an antibody are prepared as described for the local and systemic administration. The resulting mixture can be a solution, suspension, emulsions or the like and can be formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

An anti-FAM19A1 antibody described herein can be formulated as an aerosol for topical application, such as by inhalation (see, e.g., U.S. Pat. Nos. 4,044,126, 4,414,209 and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflations, alone or in combination with an inert carrier such as lactose. In such a case, the particles of the formulation will, in some aspects, have diameters of less than 50 microns, in certain aspects, less than 10 microns.

An antibody, or antigen-binding fragment thereof, described herein can be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the antibody alone or in combination with other pharmaceutically acceptable excipients can also be administered.

Transdermal patches, including iontophoretic and electrophoretic devices, are well known to those of skill in the art, and can be used to administer an antibody. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957, each of which is herein incorporated by reference in its entirety.

In certain aspects, a pharmaceutical composition comprising an anti-FAM19A1 antibody described herein is a lyophilized powder, which can be reconstituted for administration as solutions, emulsions and other mixtures. It can also be reconstituted and formulated as solids or gels. Lyophilized powder is prepared by dissolving an antibody (e.g.. anti-FAM19A1 antibody), or a pharmaceutically acceptable derivative thereof, in a suitable solvent. In some aspects, the lyophilized powder is sterile. The solvent can contain an excipient which improves the stability or other pharmacological component of the powder or reconstituted solution, prepared from the powder. Excipients that can be used include, but are not limited to, dextrose, sorbitol, fructose, corn syrup, xylitol, glycerin, glucose, sucrose or other suitable agent. Solvent can contain a buffer, such as citrate, sodium or potassium phosphate or other such buffer known to those of skill in the art at, in some aspects, about neutral pH. Subsequent sterile filtration of the solution followed by lyophilization under standard conditions known to those of skill in the art provides the desired formulation. In some aspects, the resulting solution will be apportioned into vials for lyophilization. Each vial will contain a single dosage or multiple dosages of the compound. The lyophilized powder can be stored under appropriate conditions, e.g., at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

The antibodies, or antigen-binding fragments thereof, the bispecific molecule, or the immunoconjugate described herein and other compositions provided herein can also be formulated to be targeted to a particular tissue, receptor, or other area of the body of the subject to be treated. Many such targeting methods are known. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874, each of which is herein incorporated by reference in its entirety.

The compositions to be used for in vivo administration can be sterile. This is readily accomplished by filtration through, e.g., sterile filtration membranes.

### XI. Kits

Provided herein are kits comprising one or more antibodies described herein, or antigen-binding fragments thereof, wherein the kits are for diagnostic or treatment. In certain aspects, provided herein is a pack or kit comprising one or more containers filled with one or more of the ingredients of the compositions described herein, such as one or more antibodies provided herein or an antigen-binding fragment thereof, optional an instructing for use. In some aspects, the kits contain a composition described herein and any diagnostic, prophylactic or therapeutic agent, such as those described herein.

### EXAMPLES

### Example 1 Anti-FAM19A1 Antibody Screening

The phage-scFv antibody library from Y-Biologics (Daejon, Korea) consists of 10 different library sets with 1-3×10¹⁰ diversity, forming a total of 1×10¹¹ diversity. To screen for the relevant antibodies, bio-panning was performed. Briefly, FAM19A1-Fc and FAM19A1-mFc proteins (Y-Biologics, Daejon, Korea) were used to coat immunosorb tubes and then blocked. The library phage was prepared by culturing human scFv library cells (with 10¹⁰ diversity) for 16 hours at 30° C. after phage infection, concentrating with PEG, and then suspending in PBS buffer. The library phage was then added to the immunosorb tubes and incubated for 2 hours at room temperature. After the incubation, the tubes were washed with 1×PBS/T and 1×PBS and only those scFv phages bound to the antigen (FAM19A1 protein) were eluted. The pool of positive phages were used to infect E. coli for additional amplification and bio-panning. Bio-panning was performed for up to a total of three rounds by repeating the above process. With each amplification, the phages were screened and selected for high affinity to the FAM19A1 protein.

### Example 2 Selection of Anti-FAM19A1 Antibody Clones

To investigate the specificity of the positive poly scFv-phage antibody pool from each round of bio-panning, a poly-phage ELISA was performed. ELISA immuno-plates were coated with ITGA6-Fc protein or FAM19A1-4-Fc proteins. Then, the phage antibody pool from Example 1 were added to the plates and direct ELISA was performed. A M13 phage #38 (directed to a non-displayed antibody) was used as a negative control.

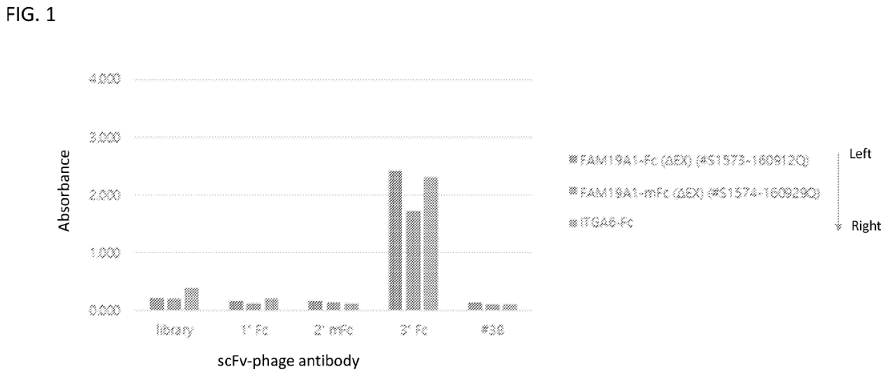

As shown in FIG. 1, scFv-phage antibody pool from round 3 of bio-panning was successfully enriched with anti-FAM19A1 phage antibody.

Next, based on the poly-phage ELISA results, approximately 1000 mono clones were selected from round 3 of bio-panning that showed high binding capacity. These mono clones were cultured in a 96-well plate and infected with a helper phage. Then, the mono scFv-phages were transferred to an immuno-plate coated with FAM19A1-Fc protein and direct ELISA was performed. To show that the binding was specific to FAM19A1, immuno-plate coated with ITGA6-Fc protein (non-specific antigenic control) was also used.

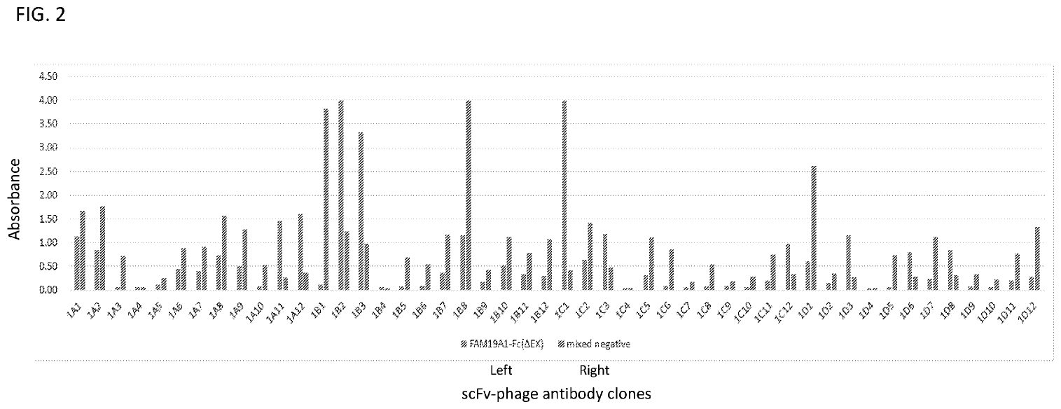

As shown in FIG. 2, the mono scFv-phage clones were found to bind only to FAM19A1-Fc, confirming the specificity of the scFv-phage clones.

Next, to group the selected positive scFv-phage clones, colony PCR was performed using a set of primers capable of amplifying scFv. The amplified PCR samples were treated with BstNI, and then the diversity of the antibodies was assessed by running the samples on a 8% DNA polyacrylamide gel.

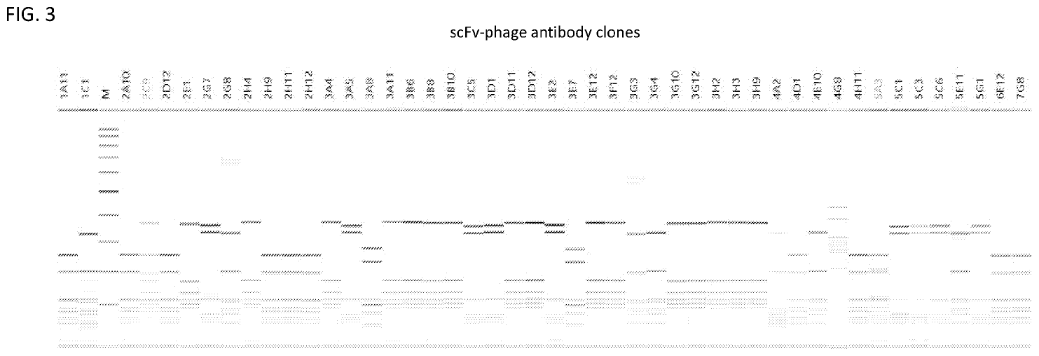

As shown in FIG. 3, based on the PCR fragmentation results, the positive scFv-phage clones were able to be divided into seven groups, which were all previously shown to bind strongly to FAM19A1-Fc but not to ITGA6-Fc.

Next, to confirm that the sc-Fv phage from each of the seven groups did not bind to other antigen, additional ELISA binding assay (as described above) using additional antigens (C-Fc, hRAGE-Fc, CD58-Fc, ITGA6-Fc, and AIRTR) was performed.

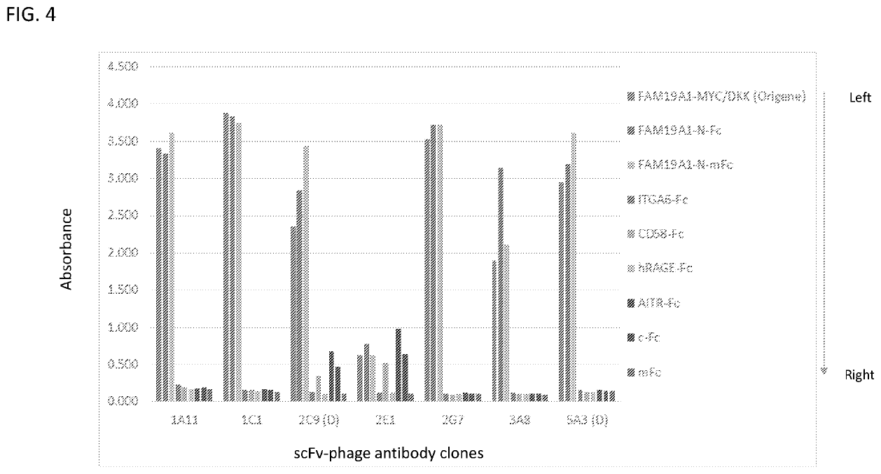

As shown in FIG. 4, of the seven clones tested, clones 1A11, 1C1, 2G7, and 3A8 showed the least binding to the non-FAM19A1 antigens. Sequence analysis showed that these four clones all had unique amino acid sequences.

### Example 3 Production of Anti-FAM19A1 IgG1 Antibody

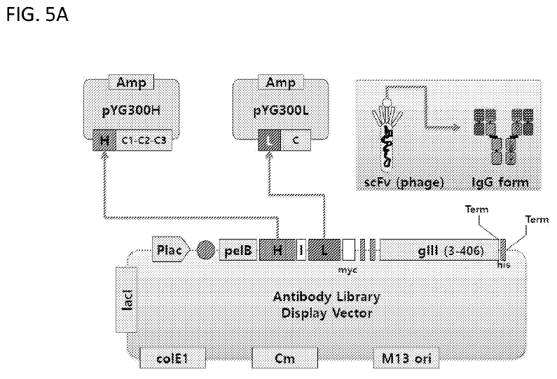

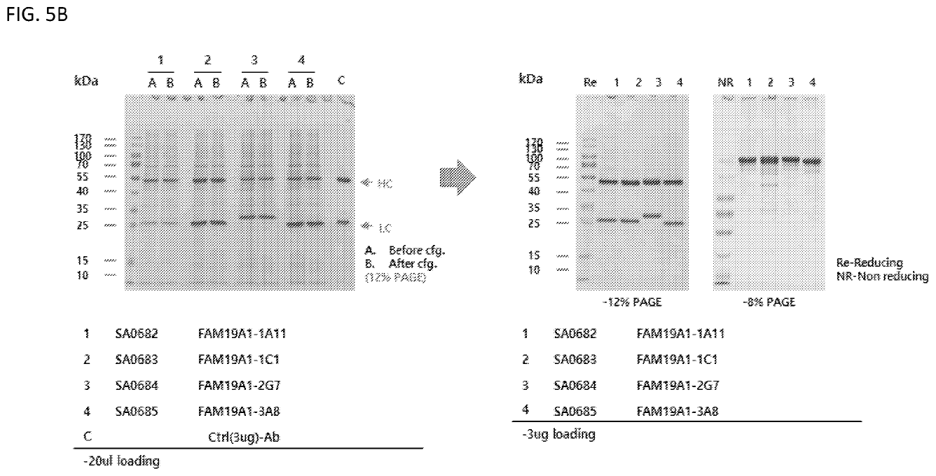

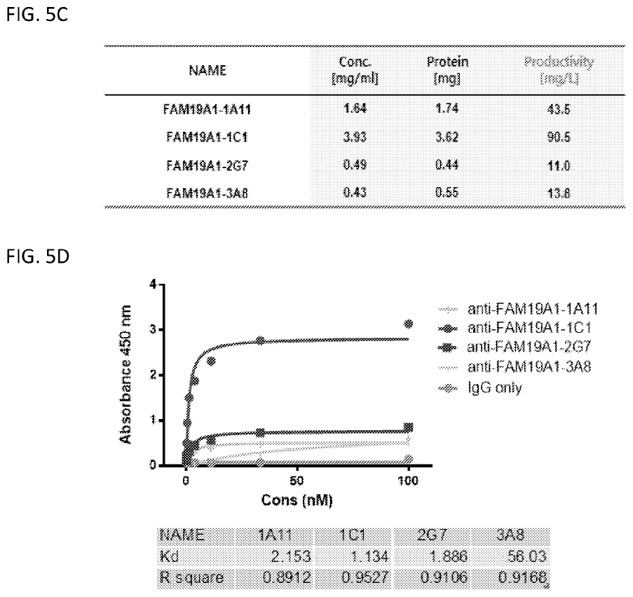

To convert the four selected monoclonal phage antibodies from scFv to human IgG, the variable region of the heavy and light chains of each of the phage antibodies was sub-cloned into an expression vector containing the constant region of the heavy and light chains. See FIG. 5A. The plasmids containing the heavy and light chains were then co-transfected into HEK 293F cells for 6 days. The antibodies produced during the 6 days were then purified using protein A affinity chromatography. After purification, the antibody was separated through glycine buffer and the final resuspension buffer was changed to PBS. The purified antibody was quantitated by BCA and nano drop. The purity and mobility of the purified protein were confirmed by SDS-PAGE analysis after loading each 5 pg of each of the four antibodies under reducing and non-reducing conditions. As shown in FIG. 5B, the four anti-FAM19A1 antibody clones had a size of about 150 kDa or more under non-reducing conditions. The productivity of the antibodies ranged from about 11 mg/L (2G7 clone) to 90.5 mg/L (1C1 clone) (FIG. 5C).

The affinity of the four anti-FAM19A1 antibody clones was also assessed using ELISA. As shown in FIG. 5D, the 1C1 clone had the greatest affinity to FAM19A1 at all concentrations tested.

### Example 4 Epitope Mapping Analysis

To further characterize the anti-FAM19A1 antibody clones, epitope mapping analysis was performed. Briefly, amino acid sequences for the different FAM19 family members (i.e., FAM19A1-5) were aligned and seven regions where the amino acid sequences of the FAM19A1 protein differed the most significantly from the other members of the FAM19A family (i.e., FAM19A2-5) were identified. The amino acid sequences of these regions were replaced with the consensus sequence of the corresponding regions for the FAM19A2-5 proteins to produce M1-M7 mutants. See Table 10

TABLE 10
Amino Acid Sequences of Wild-Type FAM19A1 and M1-M7 Mutants

| FAM19A1 | Mutations | Sequences (mutations bolded and underlined) |
|---|---|---|
| Wild Type | — | MLLCHGSLQHTFQQHHLHRPEGGTCEVIAAHRCCNKNRIEERSQTVKCS |
| | | CLPGKVAGTTRNRPSCVDASIVIGKWWCEMEPCLEGEECKTLPDNSGWM |
| | | CATGNKIKTTRIHPRT (SEQ ID NO: 44) |
| M1 | L70F | MLLCHGSLQHTFQQHHLHRPEGGTCEVIAAHRCCNKNRIEERSQTVKCS |
| | P71S | CFSGQVAGTTRNRPSCVDASIVIGKWWCEMEPCLEGEECKTLPDNSGWM |
| | K73Q | CATGNKIKTTRIHPRT (SEQ ID NO: 45) |
| M2 | N80A | MLLCHGSLQHTFQQHHLHRPEGGTCEVIAAHRCCNKNRIEERSQTVKCS |
| | R81K | CLPGKVAGTTRAKPSCVDASIVIGKWWCEMEPCLEGEECKTLPDNSGWM |
| | | CATGNKIKTTRIHPRT (SEQ ID NO: 46) |
| M3 | I91L | MLLCHGSLQHTFQQHHLHRPEGGTCEVIAAHRCCNKNRIEERSQTVKCS |
| | G92Q | CLPGKVAGTTRNRPSCVDASIVLQRWWCEMEPCLEGEECKTLPDNSGWM |
| | K93R | CATGNKIKTTRIHPRT (SEQ ID NO: 47) |
| M4 | E97Q | MLLCHGSLQHTFQQHHLHRPEGGTCEVIAAHRCCNKNRIEERSQTVKCS |
| | E103P | CLPGKVAGTTRNRPSCVDASIVIGKWWCQMEPCLPGEECKTLPDNSGWM |
| | | CATGNKIKTTRIHPRT (SEQ ID NO: 48) |
| M5 | T109V | MLLCHGSLQHTFQQHHLHRPEGGTCEVIAAHRCCNKNRIEERSQTVKCS |
| | N113L | CLPGKVAGTTRNRPSCVDASIVIGKWWCEMEPCLEGEECKVLPDLSGWM |
| | | CATGNKIKTTRIHPRT (SEQ ID NO: 49) |
| M6 | D112N | MLLCHGSLQHTFQQHHLHRPEGGTCEVIAAHRCCNKNRIEERSQTVKCS |
| | M117S | CLPGKVAGTTRNRPSCVDASIVIGKWWCEMEPCLEGEECKTLPNNSGWS |
| | A119S | CSSGHKIKTTRIHPRT (SEQ ID NO: 50) |
| | T120S | |
| | N122H | |
| M7 | R128K | MLLCHGSLQHTFQQHHLHRPEGGTCEVIAAHRCCNKNRIEERSQTVKCS |
| | I129V | CLPGKVAGTTRNRPSCVDASIVIGKWWCEMEPCLEGEECKTLPDNSGWM |
| | H130T | CATGNKIKTTKVTRRT (SEQ ID NO: 51) |
| | P131R | |

To assess binding, ELISA plates were coated with 500 ng of mutant M1-M7 or wild-type FAM19A1 protein overnight at 4° C. and then subsequently washed twice in 1×PBS. The plates were then blocked with the blocking buffer (100 μL/well) for 1 hour at room temperature. Then, the different anti-FAM19A1 antibody clones (1A11, 1C1, D6, E1, and F41H5; 1 pg) were added to the appropriate wells of the ELISA plates, and the plates were incubated at room temperature for 1 hour. After washing the plates, anti-hKappa-HRP antibody (1:2000) was added to the wells, and the plates were incubated for 30 minutes at room temperature. The color change reaction was induced by the addition of the TMB substrate. This reaction was stopped using 50 μL of sulfuric acid (2N H2SO4), and the extent of color change was detected via absorption at 450 nm with reference wavelength at 620 nm using a 96 well microplate reader (Molecular Device).

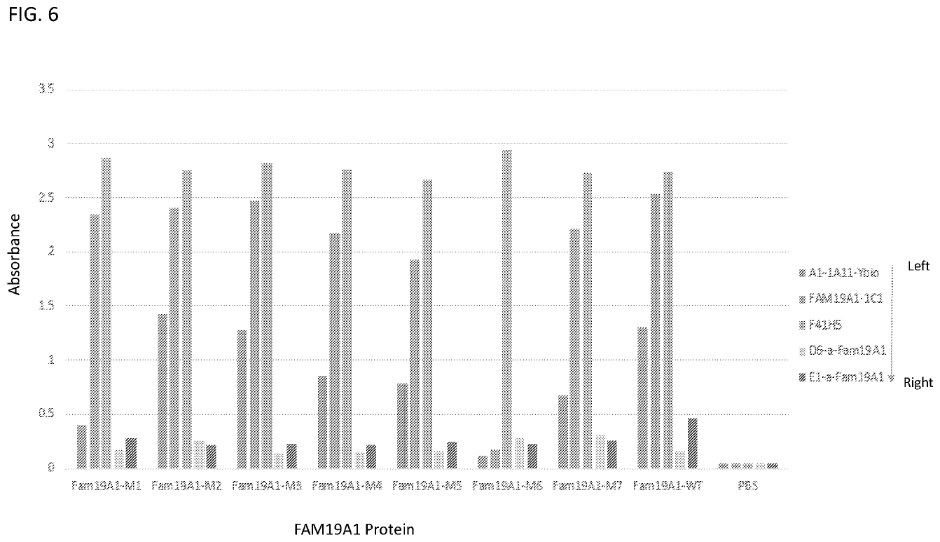

As shown in FIG. 6, anti-FAM19A1 antibody clone 1C1, which was earlier shown to have the greatest FAM19A1 binding affinity, failed to bind FAM19A1 mutant M6. As shown in Table 8 (above), the M6 mutant has substitutions at amino acid residues D112N, M117S, A119S, T120S, and N122H. This result indicates that these residues are important binding epitopes for the anti-FAM19A1 antibody clone 1C1.

### Example 5 Analysis of FAM19A1 Expression

To better characterize the expression pattern of FAM19A1, RT-PCR was used to measure FAM19A1 mRNA levels in different tissues from mice. Briefly, total RNA was isolated from both different brain regions (i.e., cerebral cortex, cerebellum, midbrain, spinal cord, hippocampus, olfactory bulb, hypothalamus, and pituitary) and peripheral tissues (i.e., heart, liver, spleen, stomach, small intestine, testis, kidney and lung). RNA was isolated using a single-step acid guanidinium thiocyanate-phenol-chloroform method as previously described. See Chomczynski, P., et al., Anal Biochem 162(1): 156-9 (1987). Then, 1 μm of each RNA sample was reverse-transcribed with Maloney Murine Leukemia Virus (M-MLV) Reverse Transcriptase (Promega, Madison, Wis.). Next, aliquots of the cDNAs were amplified using the following primers: (i) mFAM19A1_F: 5′-ATG GCA ATG GTC TCT GCA-3′; and (ii) mFAM19A1_R: 5′-TTA GGT TCT TGG GTG AAT-3′.

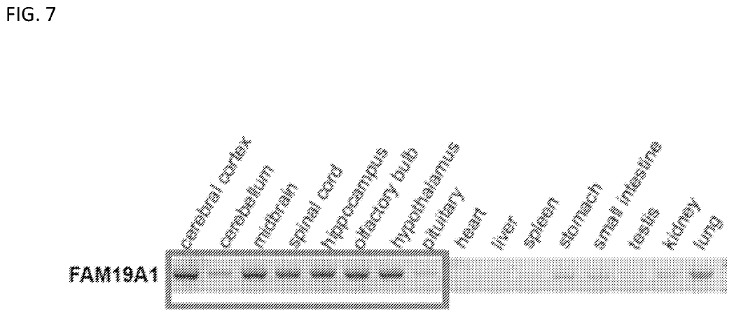

As shown in FIG. 7, FAM19A1 mRNA was observed in all brain regions tested. But, in the peripheral tissues, no or relatively low expression was observed compared to the brain regions. This result suggests that FAM19A1 is expressed primarily within the central nervous system.

### Example 6 Development of FAM19A1 LacZ Knock-In (KI) Mouse

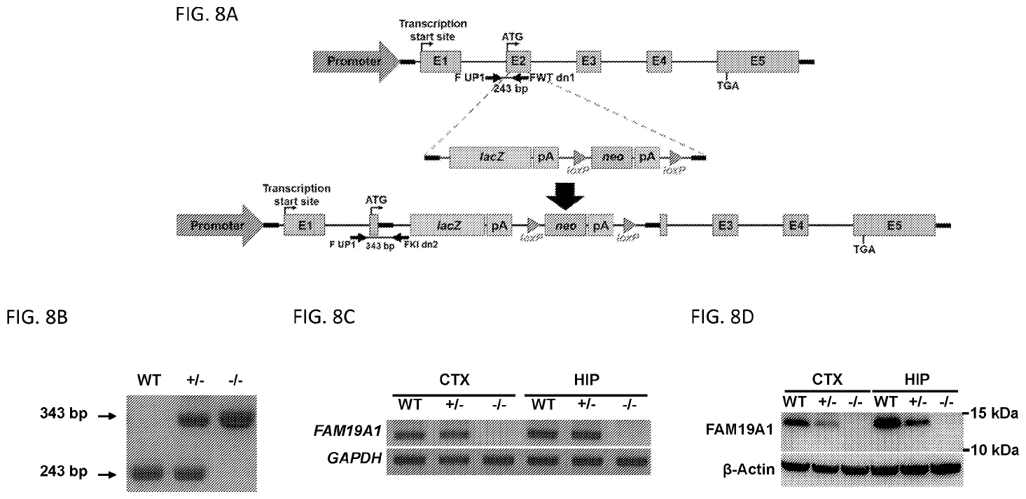

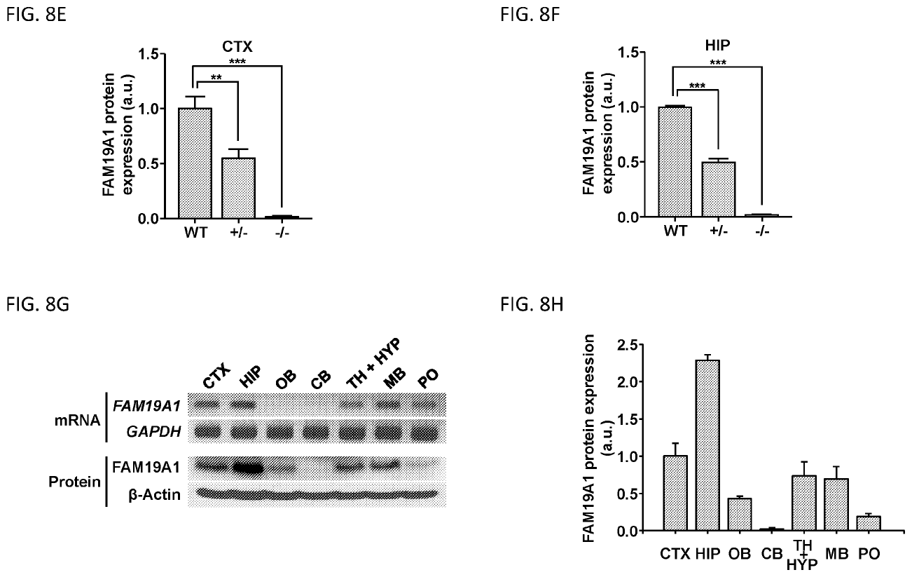

To further characterize FAM19A1 expression and function, a transgenic mouse with a lacZ reporter inserted into the FAM19A1 gene was established. Briefly, LacZ sequence containing targeting vector for FAM19A1 was constructed (FIG. 8A) and delivered to embryonic stem (ES) cells by electroporation. The target vector incorporation was validated by genotyping and chromosome counting of the transgenic ES cells. The confirmed ES cells were injected into blastocyst and transferred to uterus of a female recipient mouse. Germline transmission test was performed for stable germline expression in chimeric generation. The generated FAM19A1 LacZ KI chimeric mouse was backcrossed onto C57BL/6J genetic background. The two strains were maintained by mating heterozygous male mice with wide type C57BL/6J female mice. To obtain homozygous FAM19A1 LacZ KI mice, the heterozygous male mice were mated with the heterozygous female mice.

With the current animal model, it was expected that the insertion of the lacZ gene (inserted right after the start codon in the exon 2 of the FAM19A1 gene, see FIG. 8A) would result in beta-galactosidase expression, instead of FAM19A1, when and where FAM19A1 was supposed to be present. Therefore, it was predicted that inserting the target vector into both alleles of the FAM19A1 gene would result in complete ablation of FAM19A1 expression. Mice with the lacZ gene inserted in both alleles were designated as homozygous FAM19A1 LacZ Knock-In (“FAM19A1 LacZ KI (−/−)”).

To confirm the deleting of the FAM19A1 gene at genomic level, DNA PCR using primers specifically targeting the inserted LacZ gene sequence was used. To confirm the complete deletion of the FAM19A1 gene at the protein level in these mice, Western blot (“WB”) and Immunohistochemistry (“IHC”) were performed using polyclonal anti-FAM19A1 and/or anti-beta-galactosidase antibodies.

For the WB analysis, cortical and hippocampal areas of adult mice were isolated and lysed with a buffer containing 50 mM Tris-HCL (pH7.5), 0.1% sodium dodecyl sulfate (SDS) and a protein inhibitor cocktail tablet (Roche Applied Science). Protein contents in the lysates were quantified using BioRad Bradford protein assay reagent (BioRad) and resolved on a SDS polyacrylamide gel. The resolved proteins were transferred to nitrocellulose blotting membranes in a Bio-Rad Trans-Blot electrophoresis apparatus (Richmond, Calif.) and the blots were blocked in Tris-buffered saline containing 0.3% Tween 20 and 5% skim milk for 30 min at RT. The blots were incubated with primary antibodies for 3 hr and then incubated with horseradish peroxidase conjugated secondary antibodies for 1 hr at RT. After application of GE healthcare ECL reagents, immunoreactive bands were visualized by exposure to X-ray film. Antibodies and their dilution factors were as follows: rabbit polyclonal anti-FAM19A1 (in-lab generated) in 1:500, beta-actin (ab8227, Abcam) in 1:2000, HRP conjugated anti-rabbit (Jackson ImmunoReserch Laboratories, West Grove, Pa.) in 1:5000.

For the IHC analysis, animals were perfused with 4% paraformaldehyde in phosphate-buffered saline (PBS). The brains were isolated and post-fixed in the same fixative overnight. The brains were then cryo-protected in 30% sucrose in PBS and serially cross-sectioned in 40 μm using Cryostat (Leica). The sections were blocked with 3% BSA and 0.1% Triton X-100 in PBS for 30 min at room temperature (RT). Primary antibodies were applied overnight at 4° C. and then appropriate fluorescent conjugated secondary antibodies were applied with Hoechst 33342 (Invitrogen) for 30 min at RT. Antibodies and their dilution factors were as follows: rabbit polyclonal anti-FAM19A1 (in-lab generated) in 1:500, beta-galactosidase (ab9391, Abcam) in 1:500, fluorescent conjugated anti-rabbit and anti-chick (Life technologies) in 1:500. Images were obtained using confocal microscope (TCS SP8, Leica).

As shown in FIG. 8B, genomic DNA PCR confirmed the successful insertion of the LacZ gene in the FAM19A1 LacZ KI (−/−) animals. Given that the inserted LacZ gene sequence has its own stop codon as well as a poly-A tail, the final product of this gene construct is the intact β-galactosidase without any parts of FAM19A1. Disruption of the FAM19A1 gene in FAM19A1 LacZ KI mice was confirmed by RT-PCR (FIG. 8C). And, as shown in FIGS. 8D-8F, both in the cortex (CTX) and the hippocampus (HIP), FAM19A1 protein expression was substantially decreased in heterozygous mice (FAM19A1 LacZ KI (+/−)) compared to the wild-type animals. In the homozygous mice (FAM19A1 LacZ KI (−/−)), FAM19A1 protein was not detected. FIGS. 8G and 8H confirm that the disruption of FAM19A1 protein expression was directly related to mRNA levels. These results confirm the complete deletion of the FAM19A1 gene in the FAM19A1 LacZ KI (−/−) mice.

### Example 7 FAM19A1 Expression in Embryonic and Postnatal Mouse Brain

To better understand the function of FAM19A1, both the pattern and timing of FAM19A1 expression was assessed using X-gal staining, an enzymatic assay based on beta-galactosidase activity. Because the complete knock-out of FAM19A1 gene starting from developmental stages might cause deformations of brain structure, which could alter results, the FAM19A1 LacZ KI (+/−) (heterozygotes) animals were used.

X-gal staining for embryo, postnatal brains, and adult eyes: For embryonic X-gal staining, the pregnant mouse were sacrificed by cervical dislocation and the embryos were isolated. Whole embryos, E12.5 were fixed in 4% paraformaldehyde and 0.2% glutaraldehyde in PBS for 15 min at 4° C. For embryos older than E14.5, the heads were cut and the skins were removed. The heads of embryos were fixed in the same fixative for 1˜2 hr at 4° C. For the postnatal brains and the adult eyes, the brains and eyes were isolated from the skulls and fixed in the same fixative for 1-2 hr at 4° C. The fixed tissues were then washed with PBS twice for 5 min and incubated in stained overnight in X-gal staining solution, 1 mg/ml of X-Gal, 2 mM MgCl2, 5 mM EGTA, 5 mM potassium ferrocyanide, 5 mM potassium ferricyanide, 0.01% sodium deoxycholate, and 0.02% Nonidet-P40 in 0.1 M phosphate buffer at pH 7.3 for 24-48 hr at 37° C. in dark. The stained tissues were post-fixed with 4% paraformaldehyde in PBS overnight at 4° C., and washed, then whole brain images were obtained.

For x-gal stained sections, the stained whole brains or eyes were cryo-protected with 30% sucrose in PBS and sectioned at 40 μm using Cryostat (Leica). Nuclear Fast Red (H-3403, VECTOR) was used as a counter-stain where appropriate. The images of the sections were taken using slide-scanner (Axio scan Z1, Zeiss).

X-gal staining for adult brains: Animals were perfused with 4% paraformaldehyde and 0.2% glutaraldehyde in phosphate buffer (PB). The brains were isolated and post-fixed in 0.2% glutaraldehyde in PB for 24 hr at 4° C. The brains were then cryo-protected in 30% sucrose in PBS and serially cross-sectioned in 40 μm using Cryostat (Leica). The sectioned tissues were then incubated in X-gal staining solution, 1 mg/ml of X-Gal, 2 mM MgCl2, 5 mM EGTA, 5 mM potassium ferrocyanide, 5 mM potassium ferricyanide, 0.01% sodium deoxycholate, and 0.02% Nonidet-P40 in 0.1 M phosphate buffer at pH 7.3 for 24-48 hr at 37° C. in dark. The images of the sections were taken using slide-scanner (Axio scan Z1, Zeiss).

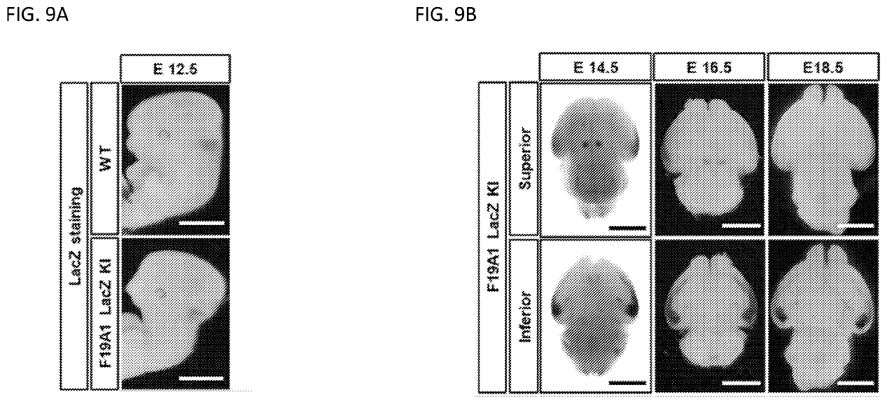

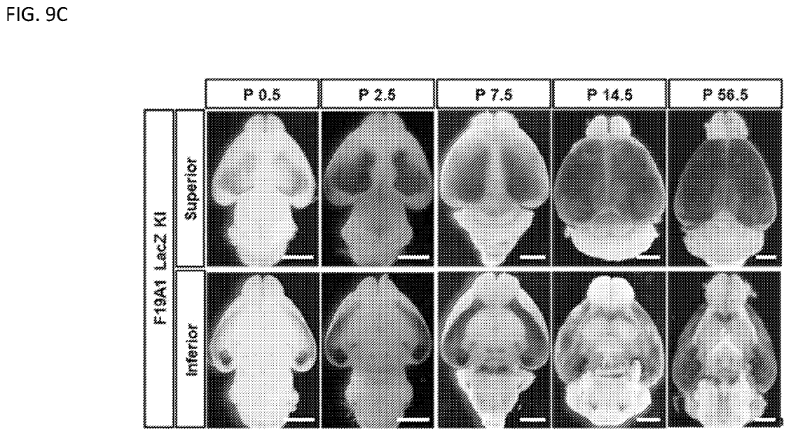

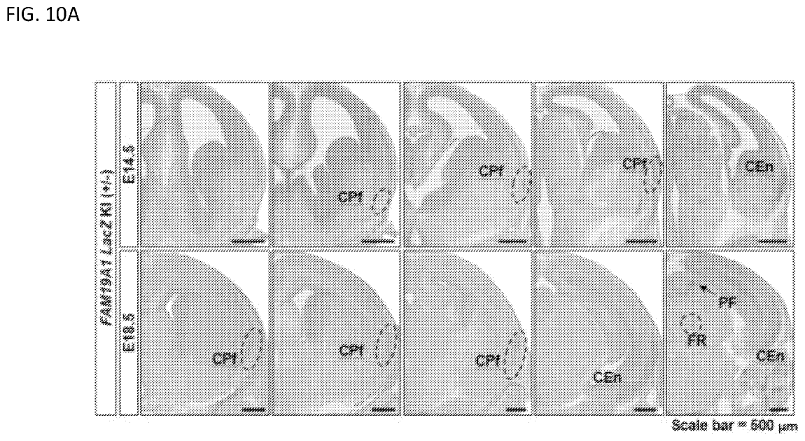

As shown in FIGS. 9A and 9B, FAM19A1 was initially expressed (as evidenced by positive beta-galactosidase staining) in limited cortical areas during early embryonic development. There was no sign of FAM19A1 expression through embryonic day 12.5 (E12.5). However, starting from embryonic day 14.5 (E14.5), β-galactosidase activity (i.e., FAM19A1 expression) was observed in the ventrolateral cortical areas with particularly strong expression in the rostral parts. These stained areas were thought to be premature piriform cortex (Cpf) and entorhinal cortex (Cen). FIG. 10A.

After birth, the neocortical expression of FAM19A1 became more apparent. FIG. 9C. During the early postnatal days, FAM19A1 was initially observed in somatosensory, visual and auditory cortical areas with continuous expression in piriform cortex and entorhinal cortex. With increased time, FAM19A1 expression expanded to other neocortical areas. By postnatal day 14.5 (P14.5), neocortical β-galactosidase (i.e., FAM19A1) expression was found in a cortical layer-specific manner. FIG. 10B. In addition, X-gal staining signals were detected in the limbic areas, including the posteromedial cortical amygdaloid nucleus (PMCo), the hippocampus and the amygdala. FIG. 10B. These results suggest that FAM19A1 is likely expressed in differentiated neuronal cells during neurodevelopment, as FAM19A1 expression patterns were specifically confined to the cortical layers and regions of the limbic system. In addition, FAM19A1 was not detected in stem-cell rich regions, such as the ventricular zone or the subventricular zone, suggesting that FAM19A1 may not be involved in the proliferation of NSCs.

### Example 8 FAM19A1 Expression in Adult Mouse Brain

To assess whether FAM19A1 plays a role in neural activity, the expression pattern of FAM19A1 was mapped in adult FAM19A1 LacZ KI heterozygous mice.

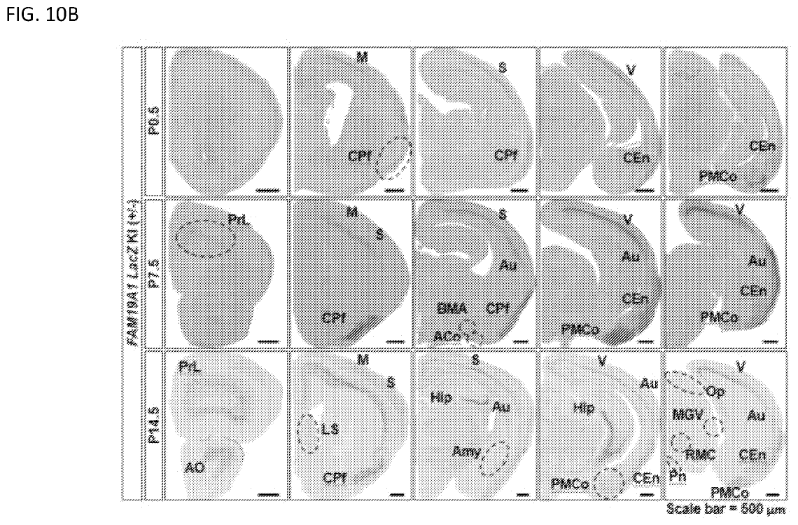

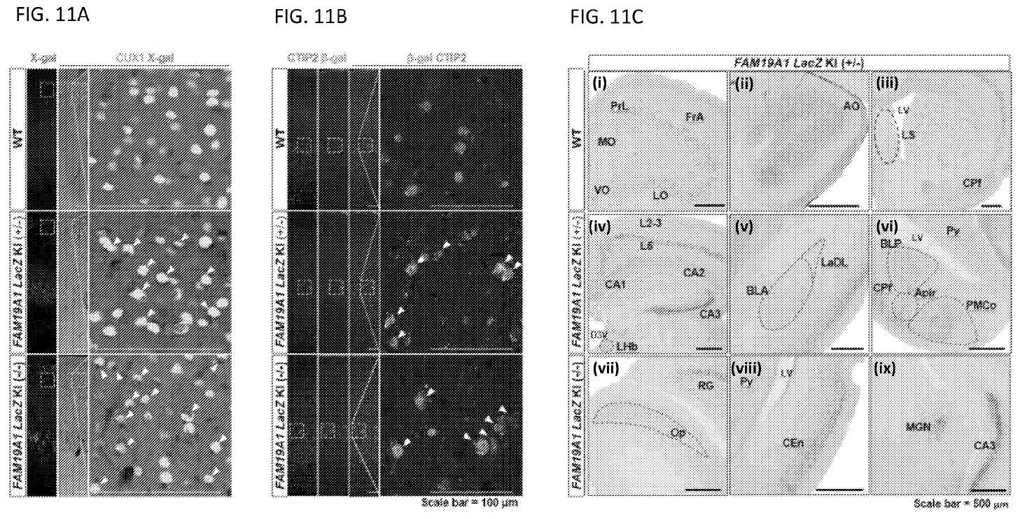

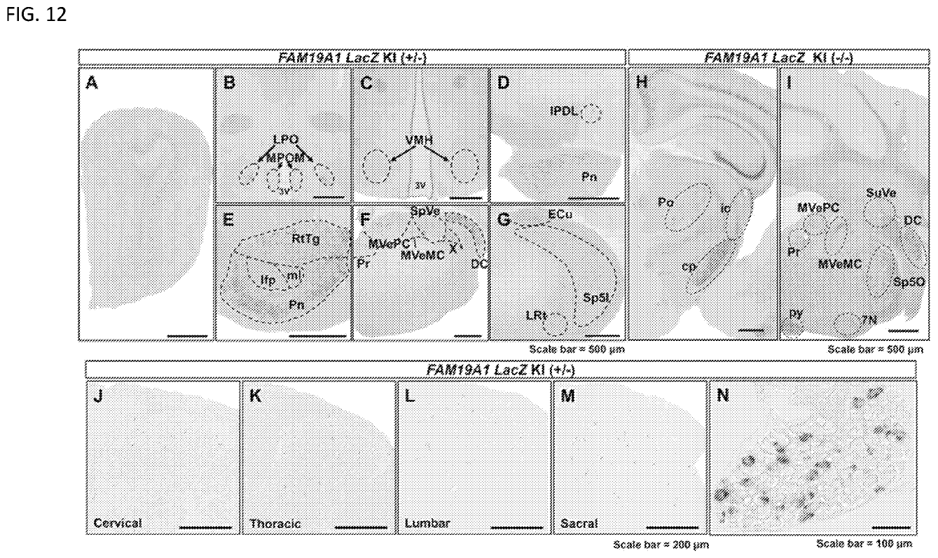

In the adult mouse brain, X-gal staining indicated that FAM19A1 was expressed in all cortical areas (FIG. 9C). Immunohistochemistry with X-gal staining showed that X-gal precipitates and β-galactosidase were co-localized with CUX1, a pyramidal neuronal marker for cortical layers 2-3 (L2-3) and CTIP2, a pyramidal neuronal marker for cortical layer 5b (L5b), respectively. This indicated that FAM19A1 is expressed mainly in pyramidal neurons in a layer-specific manner (FIGS. 11A, 11B, and 11C (panel iv)). In addition, X-gal signaled in the corticospinal tract, including the internal capsule (ic), cerebral peduncle (cp) and the pyramidal tract (py), further suggesting the presence of FAM19A1 in the pyramidal neurons of primary motor cortical L5b (FIG. 12, panels G and I).

The presence of FAM19A1 in specific sensory circuits was also investigated. In the olfactory neural circuitry, β-galactosidase and FAM19A1 mRNA expressions were hardly observed in the olfactory bulbs (OB) (FIG. 8F), but FAM19A1 protein was detected by western blotting (FIGS. 8F and 8G). The detected FAM19A1 protein could have been released from neurons of other olfaction-related brain regions, including the anterior olfactory nucleus (AO), the CPf, and the cortical amygdala that exhibited positive X-gal signals (FIGS. 8D, 8E, and 8H). For the visual neural circuit, there was no β-galactosidase expression in the optic chiasm or the lateral geniculate nucleus (LGN) of the visual neural circuit, but the optic layer of the superior colliculus (Op) and the visual cortex both exhibited β-galactosidase expression (FIG. 11C, panel vii; FIG. 9C), indicating that FAM19A1 could be involved in superior colliculus-dependent visual information processing and ocular motor control. The β-galactosidase expression was also observed in some regions associated with the auditory neural circuit, including the medial geniculate nucleus (MGN), the dorsal cochlear nucleus (DC), and the auditory cortex (FIG. 11C, panel ix; FIG. 12, panel E).

There was notable expression of FAM19A1 in the limbic regions, including the hippocampus and the amygdala. In the hippocampus, the β-galactosidase was expressed in the CA regions but not in the dentate gyrus (DG) (FIG. 11C, panel iv). With the β-galactosidase expression in the CEn, it was possible that the hippocampal FAM19A1 expression suggested the role of FAM19A1 in the hippocampal trisynaptic circuit (FIG. 11C, panels iv and viii). Among the amygdaloidal nuclei, 0-galactosidase was expressed exclusively in the basolateral nuclei, including the lateral amygdaloid nucleus (LaDL) and the basomedial amygdaloid nucleus (BLA) (FIG. 11C, panel v). In addition, FAM19A1 expression was detected in the PMCo and the amygdalopiriform transition area (Apir) (FIG. 11C, panel vi), which are thought to have direct connections with the BLA, the Cen, and the CPf.

The β-galactosidase was also expressed in some hypothalamic nuclei, including the medial preoptic nucleus (MPOM), the lateral preoptic area (LPO) and the ventromedial hypothalamic nucleus (VMH) (FIG. 12, panels B and C). As a part of the limbic system, the hypothalamus is known to act as a mediator between the CNS and the endocrine system. Therefore, the data provided here suggest that FAM19A1 could contribute to endocrine homeostasis. The lateral septal nucleus (LS), another brain region extensively connected to the limbic areas, also exhibited β-galactosidase expression (FIG. 11C, panel iii).

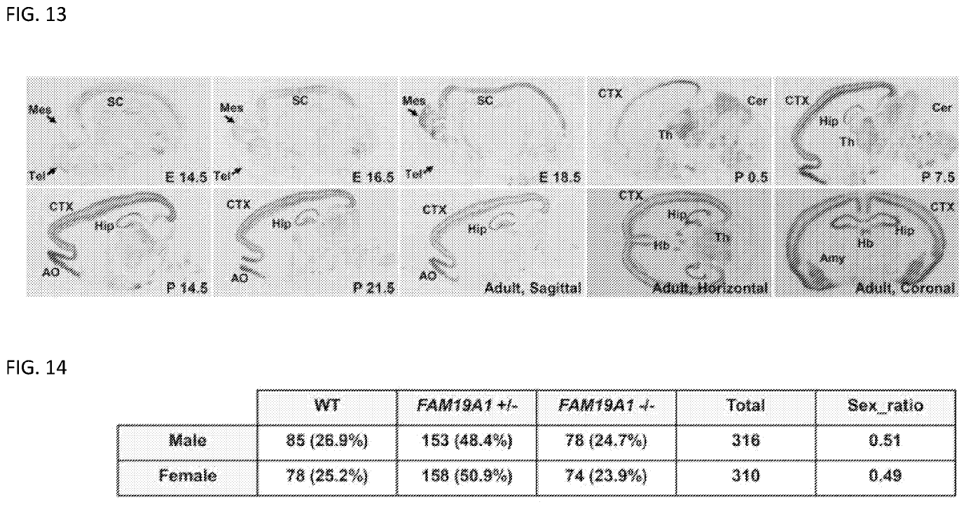

In situ hybridization using adult wild-type rat brain showed that FAM19A1 mRNA was detected in the upper and lower cortical layers, the CA regions of the hippocampus, and the basolateral nuclei of the amygdala (FIG. 13). Such FAM19A1 mRNA expression pattern coincided with the expression pattern of β-galactosidase in the FAM19A1 LacZ KI mouse brain, confirming the FAM19A1 expression mapping observed using FAM19A1 LacZ KI mice (FIG. 11C, panels ii, iv, and v). Moreover, the observed FAM19A1 expression pattern coincided with open-source single cell-based RNA-sequencing database for wild-type mouse brain. Taken together, these data indicated that FAM19A1 is mainly expressed in neurons, particularly in pyramidal neurons, and is likely to be involved in motor behavior, sensory information processing, and/or limbic system-related brain functions.

### Example 9 Comparison of Morphological Differences Between Wild-Type and FAM19A1−/− Animals

Because early deficiency of FAM19A1 could cause developmental abnormalities in the brain, morphological differences in homozygous FAM19A1 LacZ KI (FAM19A1−/−), heterozygous FAM19A1 LacZ KI (FAM19A1+/−), and WT mice were investigated.

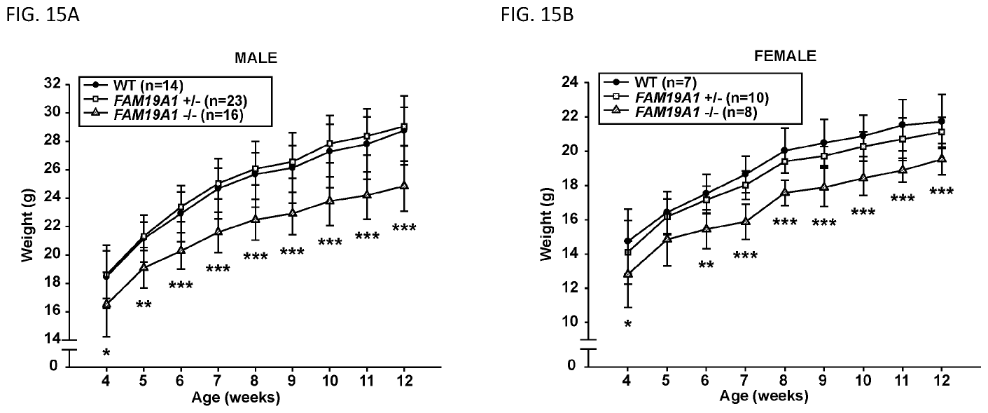

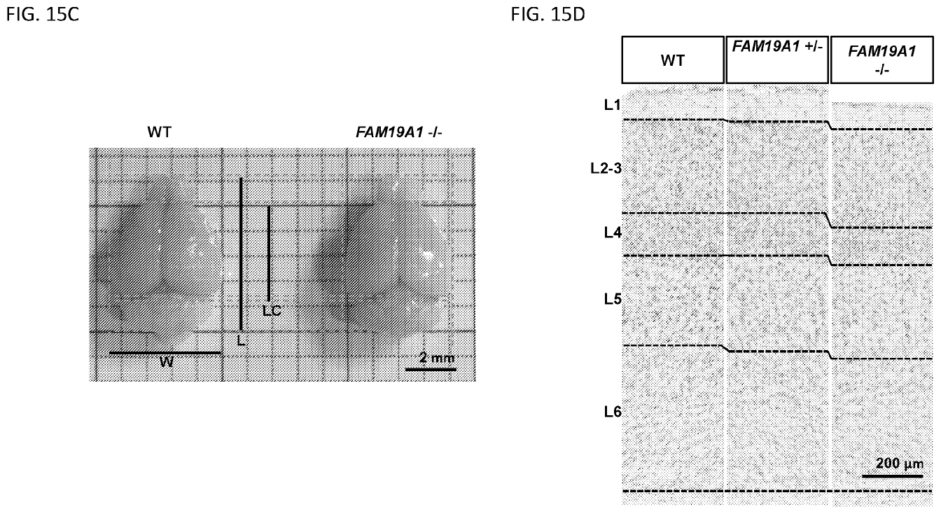

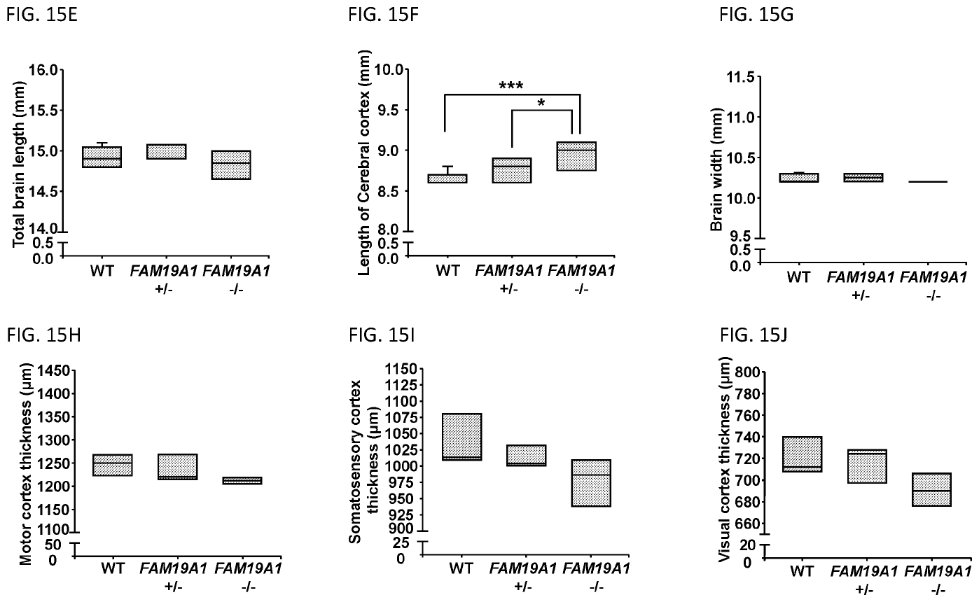

For general features, FAM19A1−/− mice were born with approximately 24-25% of Mendelian frequency and similar sex-ratio from the heterozygous parents (FIG. 14). There were no notable differences in the gross appearance between newborn genotypes immediately after birth; however, FAM19A1−/− mice (both male and female) weighed significantly less compared to the wild-type control animals (FIGS. 15A and 15B).

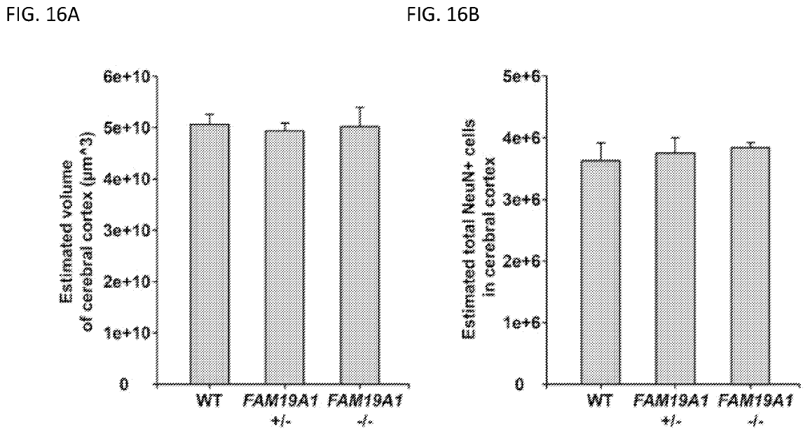

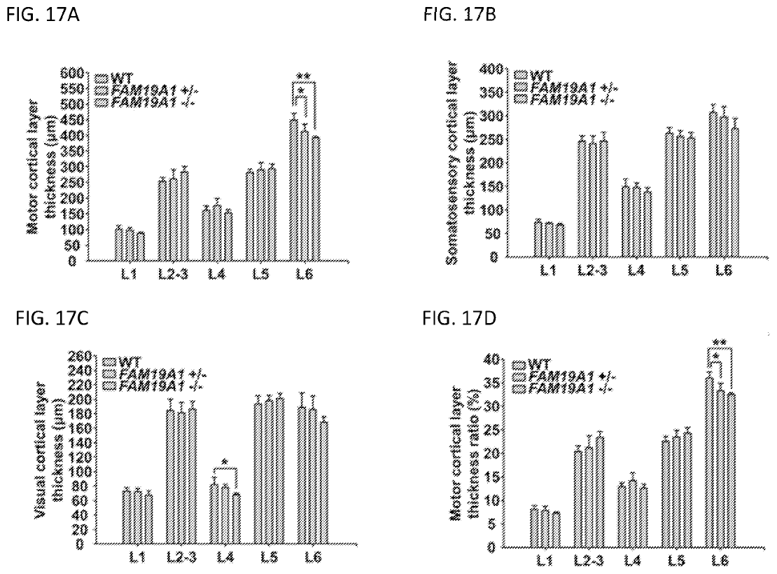

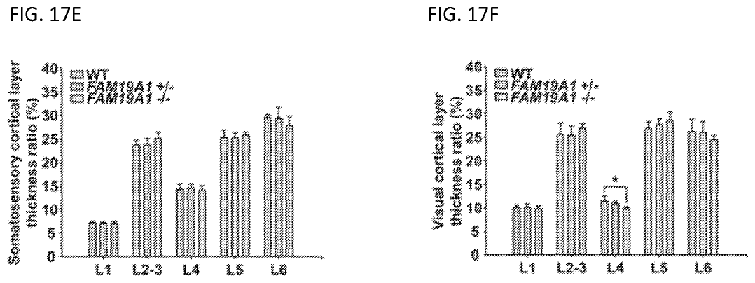

The total length and width of adult brains were similar between WT and FAM19A1−/− mice (FIGS. 15C, 15E, and 15G), whereas the cerebral cortical length was longer in FAM19A1−/− mice compared to WT mice (FIG. 15F). Ablation of genes specifically expressed in the cortical layer can lead to improper cortical layer assembly. However, in the FAM19A1−/− mice disclosed herein, cerebral cortical volume remained unaffected (FIGS. 16A and 16B). In addition, there were no notable structural abnormalities in brain architecture detected by gross observations on the X-gal stained brain sections of FAM19A1−/− mice (data not shown). The thickness of all neocortical areas was not significantly reduced in FAM19A1−/− mice (FIGS. 15H, 15I, and 15J). In terms of cortical layer proportion, however, L4 in visual cortex and L6 in motor cortex were decreased in FAM19A1−/− compared to WT mice (FIGS. 17A-17F).

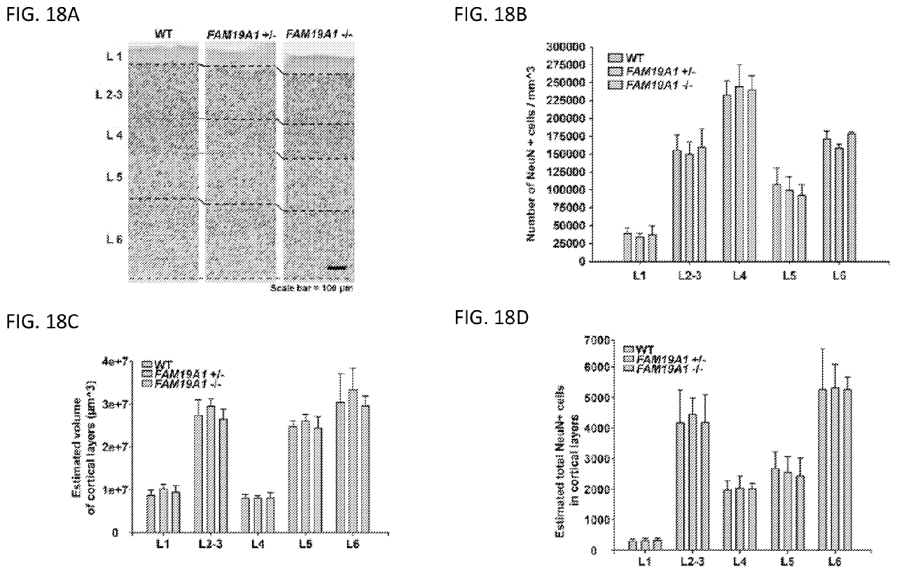

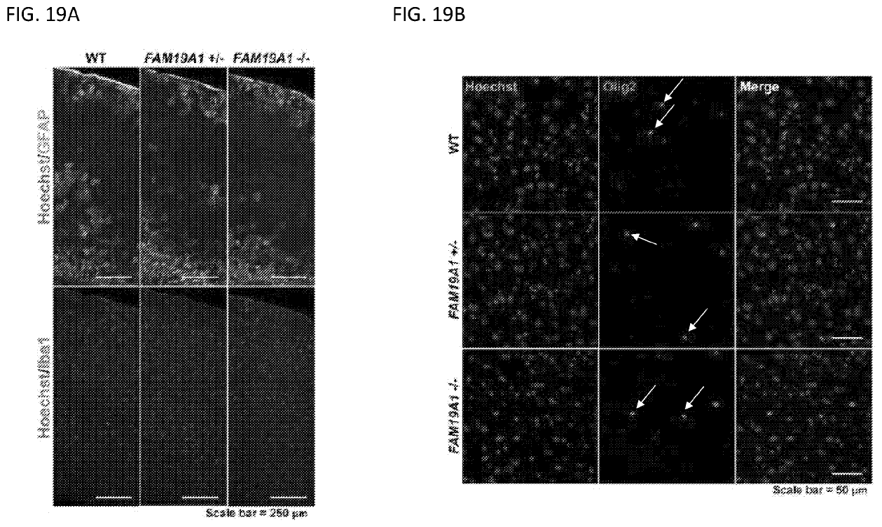

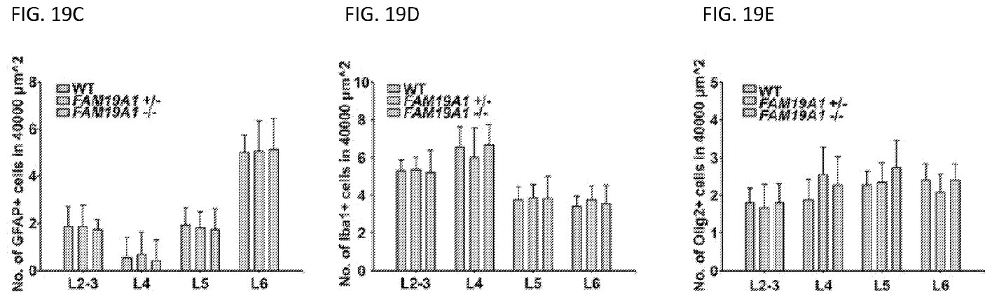

While these changes in cortical layer thickness could be the result of abnormal cytoarchitecture, no significant differences in the neuronal and glial cell populations of the cortical layers were observed between the FAM19A1−/− and WT mice (FIGS. 18A-18D and 19A-19E). In addition, there were no notable abnormalities in morphology of neuronal and glial cells (data not shown). Taken together, these findings indicate that global FAM19A1 ablation reduced body weight gain and mildly altered the neocortical architecture but did not significantly impact cell type composition of the cortex.

### Example 10 Analysis of the Role of FAM19A1 on Hyperactivity

As described earlier, FAM19A1 was expressed in several areas of the limbic system including the prelimbic cortex and the amygdala (FIG. 11C, panels i and v), which are known to be involved in emotional processing. Therefore, to evaluate the effects of FAM19A1 depletion on anxiety and depression, the elevated plus maze (EPM) test, the open field test (OFT), and the tail suspension test (TST) were performed using the FAM19A1−/− mice described above in the earlier examples. In particular, male mice were used.

EPM test was conducted as follows. The elevated plus maze (EPM) had four perpendicular arms, two open (5×30 cm) and two closed (5×30 cm), with 20-cm high walls. The maze was elevated 50 cm above the ground. The test animals were individually placed in the center of the maze facing one of the open arms and allowed to freely explore for 15 min. Recorded videos were analyzed by the ANY-maze video tracking program (Stoelting, Ill., United States). The number of open arm entries, time spent in the open arms, number of center crossings and the total distance travelled were recorded. An entry was defined as the placing of all four paws within the arm.

OFT was conducted as follows. The w40×h40×d40 cm OFT apparatus was made of opaque plastic. The test arena was defined as 30% of the central zone and the surrounding border zone. The test animals were individually placed in the center of the arena and their behavior was recorded for 10 min. The percentage of time spent and entrances into the central zone were scored, and the total distance travelled was determined using the ANY-maze video tracking program (Stoelting).

TST was conducted as follows. The mice were individually suspended by the tail in a box (36.5×30.5×30.5 cm) for 6 min. Recorded videos were analyzed by the ANY-maze video tracking program (Stoelting). Immobility was defined as the cessation of agitation and escape attempts by the mice.

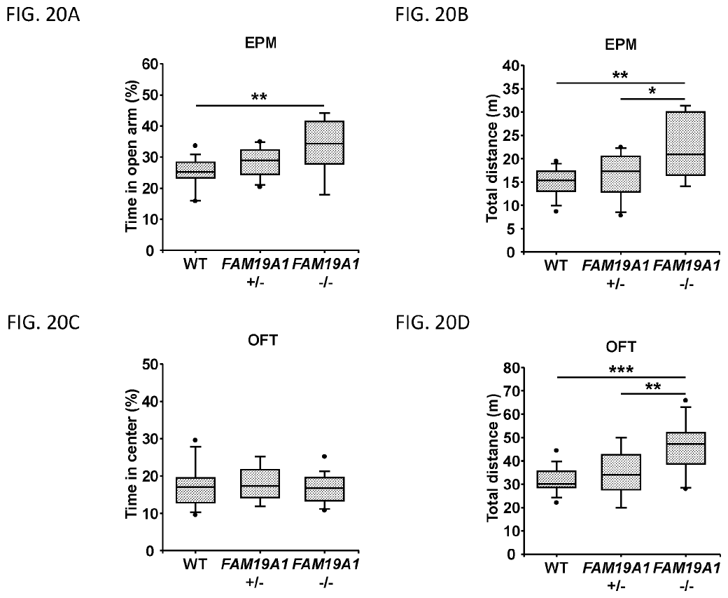

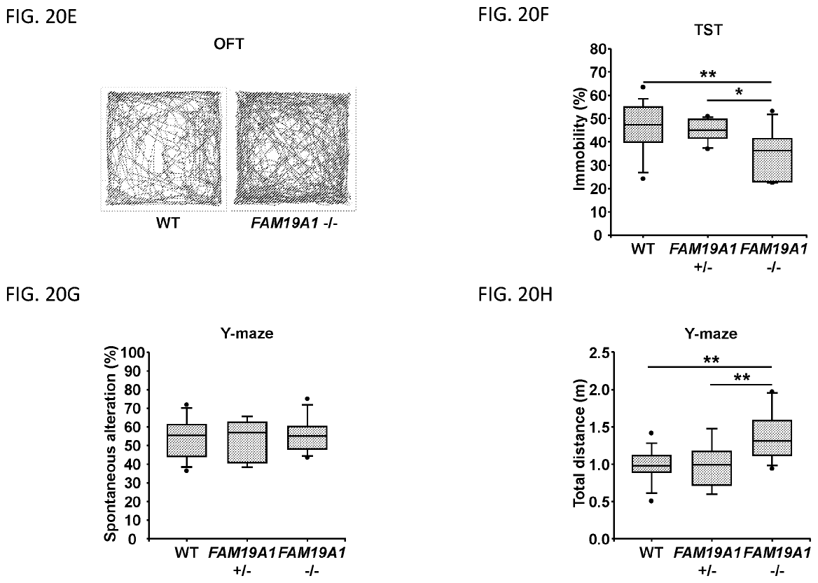

On the EPM test, the amount of time spent in the open arms (FIG. 20A) and the total distance travelled increased (FIG. 20B) in FAM19A1−/− mice compared to WT mice. On the OFT, the amount of time spent in the center of the OFT arena was similar between FAM19A1−/− and WT mice, but the total distance travelled was higher in FAM19A1−/− mice (FIGS. 20C, 20D, and 20E). On the TST, FAM19A1−/− mice displayed lower immobility than WT mice (FIG. 20F).

The above results suggest that inhibiting FAM19A1 activity can result in increased activity, which in turn could be helpful in treating anxiety or depression related disorders.

### Example 11 Analysis of the Role of FAM19A1 on Memory

Short-term memory (STM), and in particular spatial working memory, is known to involve an interaction between CA1 and CA3 of the hippocampus and the CEn. As shown in FIG. 11C (panels iv and viii), FAM19A1 is highly expressed in these areas, suggesting the possible role of FAM19A1 in memory formation. To assess the potential role that FAM19A1 might have on memory (both short-term and long-term), a Y-maze test was conducted as follows. The arena for Y-maze had three identical arms 30 cm in length and 5 cm in width with walls 20 cm high. The test animals were individually placed in the center, and the sequence of arm entries and total distance travelled over 5 min were recorded and analyzed by the ANY-maze video tracking program (Stoelting). The percentage of spontaneous alterations was calculated based on the number of trials containing entries into all three arms (ABC, ACB, BAC, BCA, CAB, CBA) divided by the maximum possible alterations (equivalent to the total number of arms entered minus 2) then multiplied by 100.

As shown in FIG. 20G, no significant differences in spontaneous alteration were observed between FAM19A1−/− and WT mice. However, the total distance travelled was significantly increased in FAM19A1−/− mice compared to the WT control animals (FIG. 20H). This result confirms the finding from the EPM and OFT tests (see Example 10), indicating that the inhibition of FAM19A1 can lead to increased activity.

Additionally, novel object recognition (NOR) tests were performed to examine possible deficits in object recognition memory. Briefly, The test arena was w40×h40×d40 cm. T-75 flasks filled with sand and stacked plastic bricks (w7×13×h15 cm) were used as objects. The mice were individually habituated in the test arena without the objects for 10 min. On the next day, during acquisition, two identical objects were placed in the arena, and the individual mouse was allowed to explore freely for 10 min. The criterion for minimal exploration time for both identical objects during the acquisition phase was 20 s. The test phase was scheduled either 6 h (for the short-term memory test) or 24 h (for the long-term memory test) after acquisition. During the test phase, both a previously introduced object and a novel object were placed in the arena. The mice then were allowed to explore the arena for 10 min. The acquisition and test phases were recorded for analysis. The time spent exploring each object was measured. Exploration behavior was defined as showing interest towards the objects by sniffing.

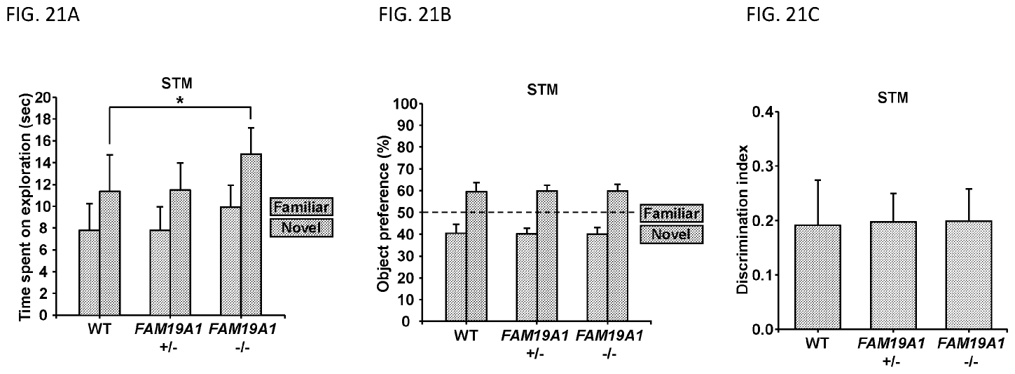

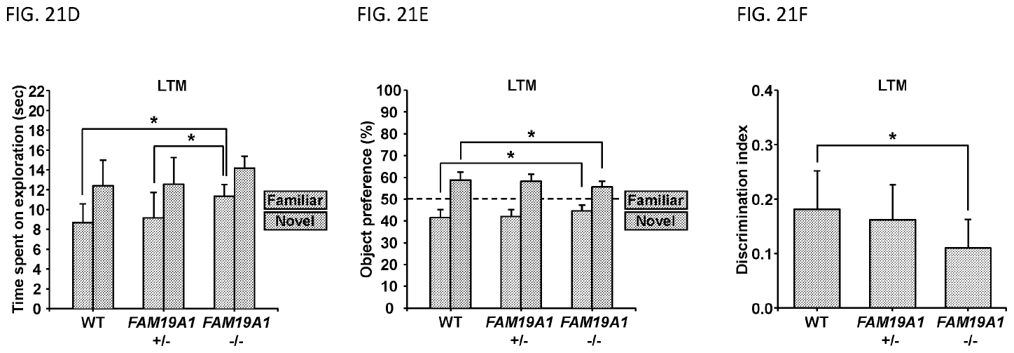

On the short-term memory version of the NOR test, there was no significant difference in preference towards novel objects between FAM19A1−/− and WT mice (FIGS. 21B and 21C). However, on the long-term memory (LTM) version of the NOR test, FAM19A1−/− exhibited lower preference towards novel objects and higher preference for familiar objects than WT mice (FIGS. 21E and 21F). These findings indicated that FAM19A1−/− mice had lower ability to discriminate between familiar and novel objects 24 hours after acquisition, indicating possible deficits in LTM in FAM19A1−/−mice. In addition, FAM19A1−/− mice tended to spend more time exploring the objects, regardless of whether the object was novel or familiar, than WT mice (FIGS. 21A and 21D).

The above results demonstrate that FAM19A1 could be important in long-term memory formation but not for short-term memory.

### Example 12 Analysis of the Role of FAM19A1 on Fear Acquisition

As shown in FIG. 11C (panels iv and v), FAM19A1 was expressed in fear processing-related areas of the brain, such as the amygdala and the hippocampus. Accordingly, to assess the potential role that FAM19A1 has on fear processing, a Pavlovian fear conditioning test was conducted using the FAM19A1−/− mice described above. Again, male FAM19A1−/− mice were used. The test was conducted as follows. The mice were habituated for 10 min in a conditioning chamber (18×18×30 cm) the day before the acquisition phase. During acquisition, the mice were placed in the conditioning chamber and underwent 5 conditioning trial repetitions, each consisting of a tone (30 s, 5 kHz, 75 dB) that terminated with foot shocks (0.7 mA, 2 s) with an inter-trial interval of 60 s. After 24 h, conditioned fear responses were tested. For the contextual test, the conditioned mice were placed in the same chamber without foot shocks and tones, and the freezing time was measured for 5 min. For the auditory test, the mice were placed in a distinct context and re-exposed to three tones at 90-s intervals without a foot shock after a 5-min period of exploration. Freezing behaviors were defined as immobility and scored during the tone presentation. The total freezing time in the test period was represented as a percentage of the average freezing duration for each tone presentation.

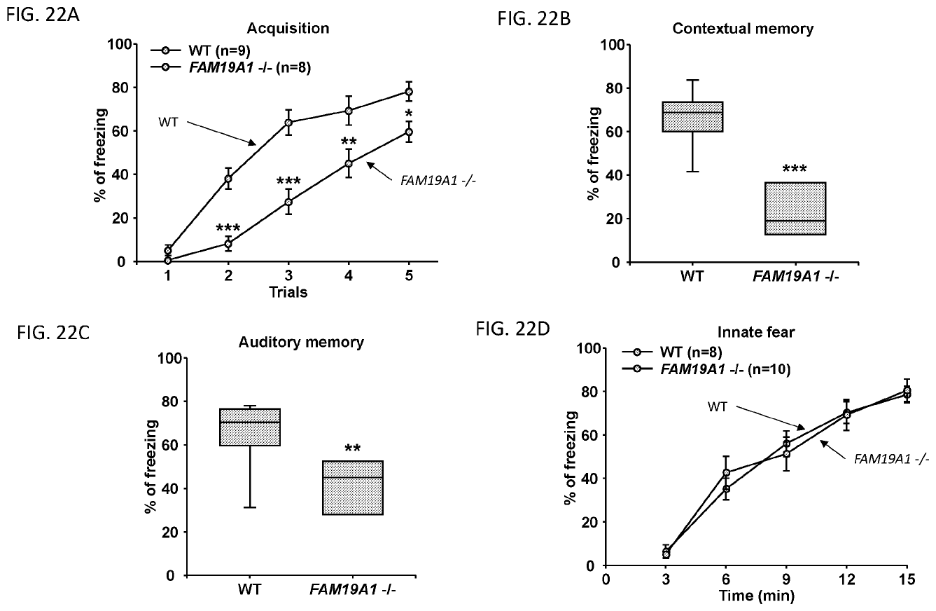

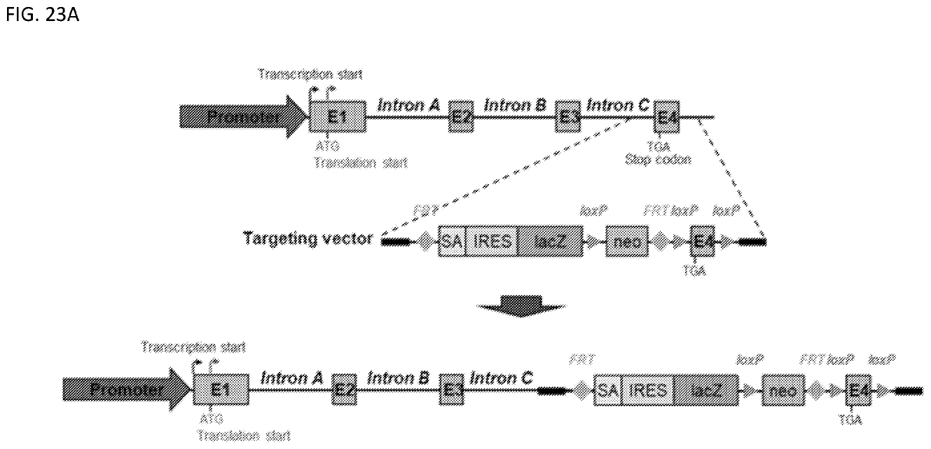

As shown in FIG. 22A, during the fear acquisition phase, FAM19A1−/− mice exhibited less freezing behavior than WT mice (FIG. 22A), indicating that fear conditioning was not properly induced in these animals. Therefore, FAM19A1−/− mice also exhibited less freezing behavior during the subsequent contextual and auditory memory tests (FIGS. 22B and 22C).

Not to be bound by any one theory, the lack of fear acquisition observed in the FAM19A1−/− animals could be related to the innate fear response. In general, mice innately experience fear when they encounter the smell of their predator's odor. To test this innate fear response, FAM19A1−/− and WT mice were placed in a chamber (18×18×30 cm) containing 30 μl synthetic predator fox feces odor (TMT, 2,5-dihydro-2,4,5-trimethylthiazoline, SRQ Bio, Florida, United States). After TMT exposure, TMT-evoked freezing behavior was recorded for 15 min and analyzed as the average percentage of freezing time at 3-min intervals using the ANY-maze video tracking program (Stoelting).

As shown in FIG. 22D, the FAM19A1−/− animals exhibited the same magnitude of fear response as WT mice over the entire TMT exposure, indicating that the innate fear response remained intact in FAM19A1−/− mice. This finding suggests that the inability of FAM19A1−/− mice to acquire a conditioned fear response is not related to the innate fear response, but instead can involve other mechanisms such as sensory dysfunctions that prevent FAM19A1−/− mice from forming an association between conditioned and unconditioned stimuli.

The above data collectively demonstrate that FAM19A1 does play a role in various CNS functions. It had previously been suggested that this role was gender dependent. For instance, Lei et al. provided data showing that only female FAM19A1−/− mice exhibit altered behavior compared to the control animals. Lei et al., FASEB J 33(12): 14734-14747 (2019). However, the data provided in the present disclosure demonstrate that FAM19A1 is also important for normal CNS functions in male subjects, as all the FAM19A1−/− mice used in the above Examples were male. Accordingly, the above Examples suggest that agents that target FAM19A1 (e.g., FAM19A1 antagonists disclosed herein) can have therapeutic effects in both male and female subjects.

### Example 13 Comparison of FAM19A1 and FAM19A5 Expression Patterns in Adult Mouse Brain

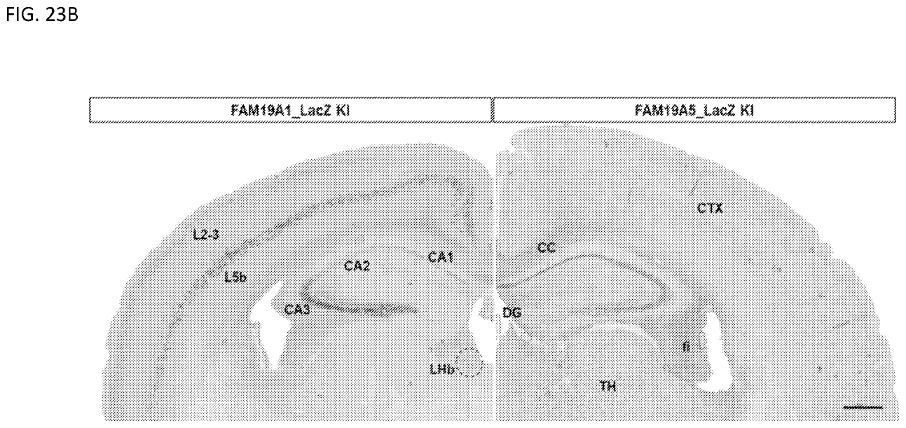

FAM19A5, another member of the FAM19A family, is also known to be highly expressed in various brain regions. U.S. Pat. No. 9,579,398 B2, which is herein incorporated by reference in its entirety. To investigate the differential expression of FAM19A1 and FAM19A5, the LacZ reporter gene system was used to develop a FAM19A5 LacZ KI mouse. Specifically, the mouse was designed to produce the fusion form of FAM19A5 and β-galactosidase (FIG. 23A).

As shown in FIG. 23B, there were key differences in FAM19A1 and FAM19A5 expression patterns. First, FAM19A1 was expressed specifically in cortical layers with a pyramidal shape, indicating that the major FAM19A1 expressing cells were pyramidal neurons. In contrast, FAM19A5 was expressed in all cortical layers, with slightly stronger intensity in L2. Corpus callosum (CC) was positive for FAM19A5 but not for FAM19A1. In addition, FAM19A5 was expressed in all hippocampal areas, thalamus and habenual, but for FAM19A1, it was only expressed in CA regions of hippocampus and lateral habenular nucleus. FIG. 23B.

These differential expression patterns indicate that FAM19A1 and FAM19A5 likely has non-overlapping functions.

### Example 14 Analysis of the Effect of FAM19A1 on Neural Stem Cell Differentiation

To further characterize the function of FAM19A1 on CNS-related function, the role of FAM19A1 on adult neural stem cell differentiation was assessed.

Neural stem cell differentiation: Briefly, adult neural stem cells (NSCs) were generated by harvesting and dissociating the subventricular zone (SVZ) of 7-9 weeks old mice brain into a single-cell suspension using 0.8% papain (Worthington, Lakewood, N.J., USA) and 0.08% dispase II (Roche Applied Science, Indianapolis, Ind., USA) in HBSS for 45 min at 37° C. In the presence of proliferative medium (containing epidermal growth factor (EGF, 20 ng/ml, Invitrogen), basic fibroblast growth factor (bFGF, 20 ng/ml, Invitrogen, Carlsbad, Calif., USA), and L-ascorbic acid (20 ng/ml, Sigma-Aldrich, St. Louis, Mo., USA)), floating colonies of cells were generated from single cells. For neurosphere differentiation assay, dissociated single cells were plated in 24-well plates at a density of 50,000 cells per well and cultured in the proliferative medium containing growth factors. At day 1, the medium was changed to differentiation medium which does not contain growth factors (i.e., epidermal growth factor (EGF, 20 ng/ml, Invitrogen), basic fibroblast growth factor (bFGF, 20 ng/ml, Invitrogen, Carlsbad, Calif., USA), and L-ascorbic acid (20 ng/ml, Sigma-Aldrich, St. Louis, Mo., USA). Then, the cells were cultured in the differentiation medium for an additional 6 days, in the presence of either anti-FAM19A1 Ab or FAM19A1 protein (500 ng/mL every day). Then, immunocytochemistry using three major neural lineages markers (i.e., Tujl, GFAP, and 04) was performed on the cells.

Immunohistochemistry: For immunocytochemical analysis, differentiated adult neural stem cells were fixed with 4% PFA at appropriate days. Cells were blocked with 3% BSA and 0.1% Triton X-100 in PBS for 30 min at RT. Primary antibodies used in this study were rabbit anti-Tuj1 (Sigma, St. Louis, Mo.), mouse anti-04 (Sigma, St. Louis, Mo.), rat anti-GFAP (Invitrogen, Carlsbad, Calif., USA). After several washes with PBS, appropriate secondary antibodies were applied for 30 min. Subsequently, the coverslips were washed, mounted, and observed under a fluorescence or confocal microscope (LSM700; Zeiss, Goettingen, Germany).

Recombinant His-FAM19A1 protein generation: For the generation of N-terminally hexahistidine-tagged FAM19A1, the gene was cloned into BamHI and XhoII sites of the expression vector pLPS-hT under control of tac promoter. The resulting plasmid pLPS-FAM19A1-6-HisN was used for transformation of E. coli DH5a. The primary structure of the cloned gene was confirmed by sequencing. Recombinant FAM19A1 was expressed in bacteria using Isopropyl β-D-1-thiogalactopyranoside (IPTG) and purified by affinity chromatography with Ni-NTA (Qiagen, Valencia, USA).

Generation of polyclonal anti-FAM19A1 antibody: For generation of anti-FAM19A1 polyclonal antibodies, antisera from rabbits immunized with synthetic FAM19A1 peptides (CHGSLQHTFQQHHLHRPEG, SEQ ID NO: 52; Abclon) were obtained. An IgG fraction was obtained from rabbit serum using a Protein A-Sepharose method (IPA-300, Repligen).

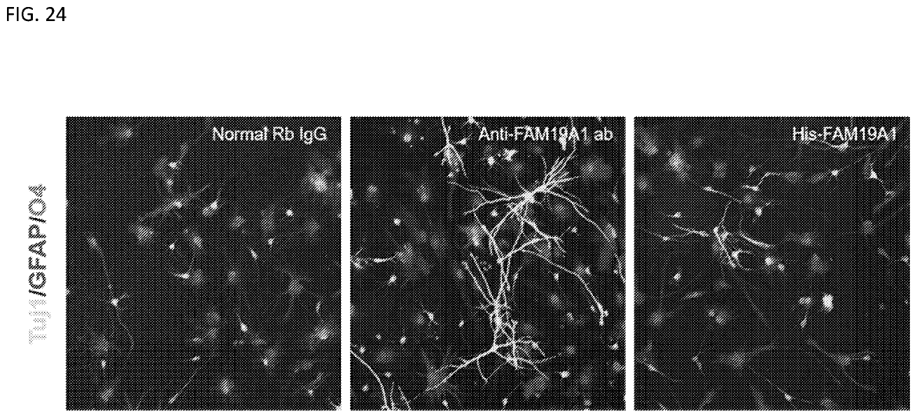

As shown in FIG. 24, NSCs treated with anti-FAM19A1 antibody had increased neurite outgrowth of differentiated neurons, compared to cells treated with control IgG antibody or FAM19A1 protein.

### Example 15 Evaluation of Intraocular Pressure after In Vivo Administration of Anti-FAM19A1 Antibody

In order to assess whether the neutralization of FAM19A1 activity in vivo could relieve elevated intraocular pressure often associated with glaucoma, a rabbit model of glaucoma was used. Briefly, New Zealand White rabbits (male, 2-2.5 kg in weight) (Hanlim Experiment Animal Research Institute, Kyonggi-Do, South Korea) were deeply anesthetized with Zoletil 50 (VIRBAC, France) and xylazine (ROMPUN®, Bayer AG, Germany). Then, the orbital cavity was exposed by raising the rabbit's eyes from the orbit. For each eye, the episcleral vein located above and below the exposed eye was cauterized using an electrocautery machine at a depth sufficient to occlude the episcleral vein without affecting the sclera and other adjacent vessels.

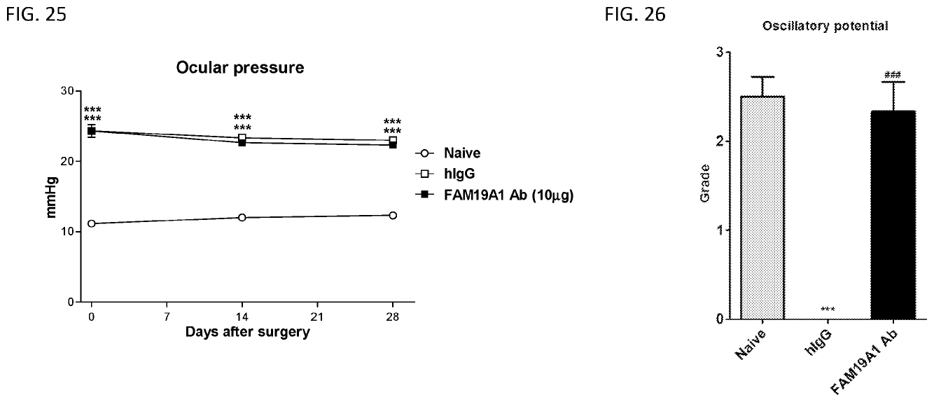

Approximately two weeks after glaucoma induction (i.e., day 0), the rabbits received either human IgG immunoglobulin (control) or anti-FAM19A1 antibody in eye via intravitreal injection (100 μg/eye in 50 μL volume). The animals received the antibodies once a week for a total of 4 weeks. Naïve (i.e., no glaucoma and no antibody injection) rabbits were used as additional control. At days 0, 14, and 28 post glaucoma induction, the intraocular pressure was measured using a TONOVET®. As shown in FIG. 25, for all time points measured, anti-FAM19A1 antibody had no effect on intraocular pressure in the glaucoma-induced rabbits. Rabbits receiving the IgG control antibody or the anti-FAM19A1 antibody had similarly elevated intraocular pressure as compared to the naïve animals.

### Example 16 Evaluation of the Retinal Potential Difference after In Vivo Administration of Anti-FAM19A1 Antibody

Electroretinogram (ERG) is a diagnostic test that measures the electrical activity generated by various cells in the retina, including the photoreceptors (rods and cones), inner retinal cells (bipolar and amacrine cells), and the ganglion cells. Generally, upon a bright stimulus flash, the ERG of a healthy eye will exhibit a complex waveform consisting of an initial negative deflection (“A-wave”) followed by a B-wave with an increasingly steeper rise and faster peak upon which are superimposed higher-frequency oscillations known as “oscillatory potentials” (OPs). The A-wave is dominated by the collective response of the rods and the scotopic B-wave by the response of rod bipolar neurons. The OPs arise within the inner plexiform layer, where the bipolar, amacrine, and ganglion cells interact. Wilsey et al., Curr Opin Ophthalmol 27(2): 118-124 (2016). By assessing these values, it may be possible to assess the health of the different cells found within the retina.

As detailed above in Example 15, glaucoma was induced in New Zealand White rabbits and the animals were injected in eye with either the human IgG control antibody or the anti-FAM19A1 antibody. At 4 weeks after the initial administration of the anti-FAM19A1 antibody, the oscillatory potential was measured to assess the electrical activity (i.e., “retinal potential difference”) within the animals' eyes.

As shown in FIG. 26, administration of the anti-FAM19A1 antibody significantly improved the oscillatory potential measured in the glaucoma-induced rabbits. In the animals that received the human IgG control antibody, no detectable oscillatory potential was measured. In contrast, the oscillatory potential in the animals that received the FAM19A1-specific antibody were comparable to that of the naïve animals. Collectively, these results, along with those from Example 15, suggest that the administration of the anti-FAM19A1 antibody to glaucoma-induced rabbits can improve retinal damage associated with glaucoma.

### Example 17 Evaluation of the Number of Retinal Ganglion Cells after In Vivo Administration of Anti-FAM19A1 Antibody

To confirm the results from the ERG analysis above, glaucoma was induced in New Zealand White rabbits and the animals were injected in each eye with either the human IgG control antibody or the anti-FAM19A1 antibody as described in Example 15. Then, at 4 weeks after the initial administration of the anti-FAM19A1 antibody, the animals were sacrificed. Both eyes from each of the animals were harvested and fixed in 10% neutral buffered formalin solution for approximately 24 hours. Afterwards, the eyes were washed with PBS and then, the cornea, lens, and the vitreous membrane were separated. Then, the eyes were made into a cup-shape and fixed in 50% methanol for 30 minutes. The eyes were next washed with sterile distilled water, and each eye was placed in a well of a 12-well plate. Approximately 0.5 mL of 0.1% ethidium bromide was added to each of the wells, and the eyes were incubated in this solution for 30 minutes. After the incubation, the eyes were washed with sterile distilled water and mounted onto a 60 mm dish. The number of cells in the retinal ganglion cell layer was measured using a fluorescence microscope.

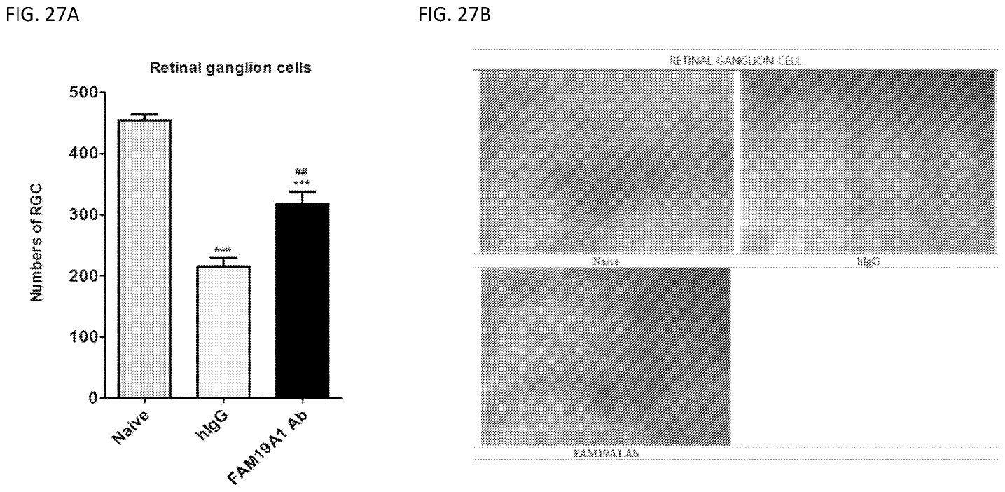

As shown in FIGS. 27A and 27B, the number of retinal ganglion cells in the glaucoma-induced rabbits that received the hIgG control antibody was significantly lower than that of the naïve control rabbits. However, in animals that received the anti-FAM19A1 antibody, there was a marked increase in the number of retinal ganglion cells observed in the retinal ganglion cell layer. Such results suggest that the administration of the anti-FAM19A1 antibody can reduce and/or restore the loss of retinal ganglion cell observed with glaucoma and protect the nerve connections of the inner plexiform layer and the retinal nerve cells.

### Example 18 Evaluation of Mechanical Allodynia after In Vivo Administration of Anti-FAM19A1 Antibody in Rat Model of Chronic Constrictive Injury

To study whether neutralization of FAM19A1 activity in vivo could relieve neuropathic pain, a rat model of chronic constrictive injury (CCI) was used as described previously by Bennett and Xie, Pain 33(1): 87-107 (1988), Austin et al., J Vis Exp 61: 3393 (2012). Experimental CCI of the sciatic nerve is one of the most widely used models for the study of neuropathic pain and has been reported to induce an inflammatory response in the ipsilateral hind paw. Accordingly, hind paw withdrawal threshold, as measured, e.g., with a Von Frey test, serves as a good indicator of neuropathic pain.

Induction of neuropathic pain by chronic constriction injury: Briefly, 6-week old male Sprague-Dawley rats were deeply anesthetized with Zoletil 50 (VIRBAC, France) and xylazine (ROMPUN®, Bayer AG, Germany). Then, the hair of the rat's lower back and thigh were shaved, and the skin was sterilized with povidone iodin. Next, the skin of the lateral surface of the thigh was incised and the common sciatic nerve was exposed at the level of the middle of the thigh by blunt dissection through biceps femoris. Proximal to the sciatic nerve about 7 mm of nerve was freed of adhering tissue and 4 ligatures (4.0 black silk) were tied loosely around it with about 1 mm spacing. The length of nerve thus affected was 4-5 mm long. After performing nerve ligation, muscular and skin layer were immediately sutured in layers with thread, and topical antibiotic was applied.

Anti-FAM19A1 antibody administration: Male Sprague-Dawley rats were anesthetized using Zoletil 50 (VIRBAC, France) and xylazine (ROMPUN®, Bayer AG, Germany) and divided into three groups as shown in Table 11 (below). One group of rats with chronic constriction injury (“CCI-induced rats”) (n=5) received anti-FAM19A1 antibody (10 pg/rat in 0.1 mL volume) via intrathecal injection. The antibody was administered once a week, for a total of 2 weeks. Another group of CCI-induced rats (n=5) were used as “negative control” and received only 0.9% saline. The remaining groups of rats (n=5) were used as “sham control” (i.e., no CCI-induction and no administration). PGP-35JI

TABLE 11
Treatment Groups

| | | # of | CCI- | | |
|---|---|---|---|---|---|
| Group | Sex | Animal | Induction | Treatment | Administration |
| Sham/Naive | M | 5 | N | — | — |
| CCI-control | M | 5 | Y | 0.9% saline | 10 |
| | | | | | μg/head/week, |
| | | | | | intrathecally |
| CCI- | M | 5 | Y | Anti- | 10 |
| FAM19A1 | | | | FAM19A1 | μg/head/week, |
| | | | | Antibody | intrathecally |

Von Frey Test At days 6, 14 and 21 post chronic constriction of the sciatic nerve, the paw withdrawal threshold was assessed using the Von Frey test. The rats were placed in an apparatus with a wire mesh floor and allowed to stabilize to the environment for about 20 minutes. Then, the paw withdrawal threshold was measured by applying a Von frey filament (0.5 mm diameter), through the mesh floor, onto the plantar surface of the hind paws 3 times at 10-second intervals.

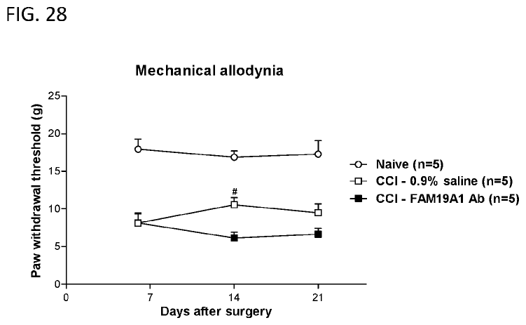

As shown in FIG. 28, CCI induction resulted in significant decrease in paw withdrawal threshold as compared to naïve animals at day 6 (baseline). Once a week (day 7 and day 14) intrathecal injection of anti-FAM19A1 antibody after CCI induction resulted in significantly increased paw withdrawal threshold compared to the 0.9% saline treated rats after CCI induction. This increase was most notable at day 14 post CCI induction. These results indicate that neutralizing FAM19A1 activity with in vivo administration of anti-FAM19A1 antibody can alleviate neuropathic pain.

### Example 19 Evaluation of Motor Function after In Vivo Administration of Anti-FAM19A1 Antibody in Rat Model of Chronic Constrictive Injury

To study whether anti-FAM19A1 antibody treatment could alter motor function, the motor activity of CCI-induced rats were assessed using the Rotarod test. This test serves as a good indicator of any pain or muscle weakness in the lower limbs, resulting from the CCI induction (Chen L et al 2014, Vadakkan K I et al 2005). CCI induction and anti-FAM19A1 antibody administration were carried out as described in Example 18.

Rotarod Test: Each Sprague-Dawley rat (sham control and CCI-induced) was carefully placed on the Rotarod-treadmill (Biological Research Apparatus 7750, UGO BASILE Inc., Italy) and the rotation speed of the rotarod was increased at regular intervals from 4 rpm to 20 rpm. The latency to fall off was recorded at days 6 (baseline). Motor performance was considered as the latency to fall off the rotarod apparatus determined from the mean time in three trials for each rat at each time.

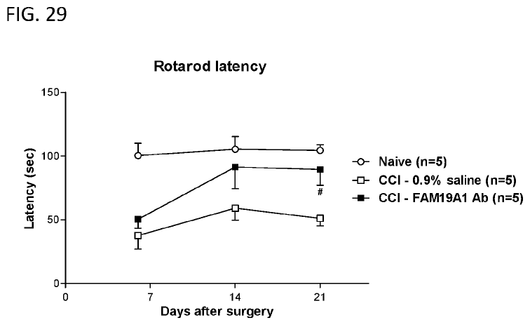

As shown in FIG. 29, CCI induction resulted in significant decrease in latency time (i.e., rats jumped off the rotarod much more quickly) compared to naïve animals at day 6 (baseline). Once a week (day 7 and day 14) intrathecal injection of anti-FAM19A1 antibody after CCI-induction resulted in improved latency time (i.e., rats remained on the rotarod apparatus longer) compared to the 0.9% saline treated group after CCI induction. These results indicate that in vivo administration of anti-FAM19A1 antibody after CCI-induction can not only reduce neuropathic pain but also improve motor function.

### Example 20 Analysis of the Effect of FAM19A1 on the Differentiation of Primary Mouse Hippocampal Neurons

To further understand the role of FAM19A1 on CNS-related function, a monoclonal anti-FAM19A1 antibody was generated (A1-1C1). Then, the antibody was used to confirm the effects of FAM19A1 on the differentiation of neurons observed earlier (see Example 14). Hippocampal cells derived from postnatal day 1 (P1) C57BL/6 mice were prepared as previously described. Chang, S., et al., Nat Neurosci 4(8):787-93 (2001). Briefly, hippocampi were dissected, dissociated with 0.25% trypsin EDTA (TE) for 10 min at 37° C., and triturated with a polished half-bore pasteur pipette in Trituration solution (1 mM L-glutamine, 10% fetal bovine serum, 10% BSA and 0.5% DNAse in HBSS). The cells (2.5×10⁵) were plated on poly-D-lysine-coated glass coverslips in a 60-mm Petri dish in minimum Eagle's medium (MEM) supplemented with 0.5% glucose, 1 mM pyruvate, 1.2 mM L-glutamine and 12% fetal bovine serum. Four hours after plating, the medium was replaced with Neurobasal media (Invitrogen) supplemented with 2% B-27 and 0.5 mM L-glutamine. Cells were maintained at 37° C. in a 5% C02-humified incubator in the presence or absence of 1 pg/ml anti-FAM19A1 antibody (human FAM19A1 1C1 monoclonal antibody).

For Immunocytochemistry, day in vitro (DIV) 3 cells were fixed in 4% formaldehyde, 4% sucrose, PBS for 15 minutes. Primary antibodies used in this study were rabbit beta-tubulin 3 (Sigma, St. Louis, Mo.). Secondary antibodies were linked to horseradish peroxidase (Jackson ImmunoReserch Laboratories, West Grove, Pa.). The morphological analysis of cortical and hippocampal neurons was determined as previously described. Baj, G., et al., Front Cell Neurosci 8:18 (2014). After the neurons were stained with beta-tubulin 3, total neurites length, number of primary and higher order neurites, and number of branch points were measured by simple neurite tracer plugin using the Image J program (Fiji, NIH, Bethesda).

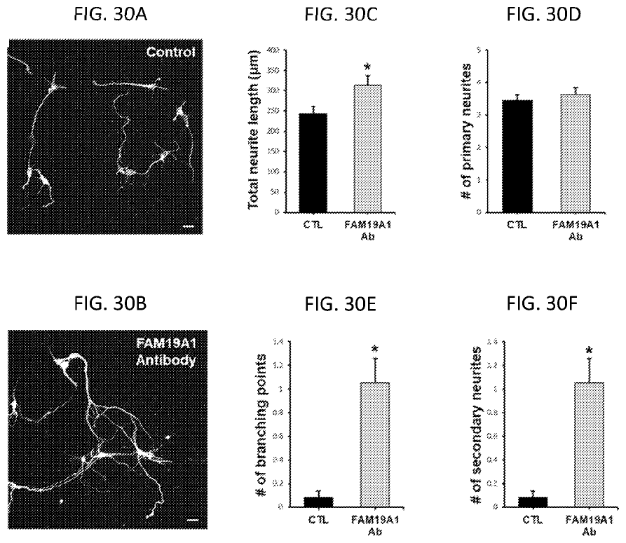

As shown in FIGS. 30A-30F, treatment of hippocampal cells with anti-FAM19A1 antibody significantly enhanced total neurite outgrowth (see FIG. 30A) compared to the vehicle-treated control cells (see FIG. 30B). Although the total number of primary neurites was not affected (see FIG. 30D), treatment with anti-FAM19A1 greatly increased the number of branching points, where secondary neurites extended from the primary neurites (see FIGS. 30E and 30F).

Collectively, the current results, along with those from Example 14, highlight the importance of FAM19A1 in regulating the differentiation of neurons.

### Example 21 Evaluation of the Analgesic Effects of a Monoclonal Anti-FAM19A1 Antibody in a Mouse Chronic Constriction Injury Model

Next, the monoclonal anti-FAM19A1 antibody (A1-1C1) was used to confirm the therapeutic effects of neutralizing FAM19A1 activity on neuropathic pain observed earlier (see Examples 18 and 19) in CCI-induced animals.

Induction of neuropathic pain by chronic constriction injury: Briefly, 8-week old male C57BL/7 mouse were deeply anesthetized with Zoletil 50 (VIRBAC, France) and xylazine (ROMPUN®, Bayer AG, Germany). Then, the lower back and thigh were shaved, and the skin was sterilized with povidone iodin. Next, the skin of the lateral surface of the thigh was incised and the common sciatic nerve was exposed at the level of the middle of the thigh by blunt dissection through biceps femoris. 3 ligatures (6.0 black silk) were tied loosely around it with about 1 mm spacing. After performing nerve ligation, muscular and skin layer were immediately sutured in layers with thread, and topical antibiotic was applied.

Monoclonal anti-FAM19A1 antibody administration: C57BU/6 mice were divided into three groups as shown in Table 12. One group of mice with chronic constriction injury (“CCI-induced mice”) (n=7) received anti-FAM19A1 monoclonal antibody (5 pg/mouse in 5 μl volume) via intrathecal injection. The antibody was administered once a week, for a total of 2 weeks. Another group of CCI-induced mice (n=6) were used as “negative control” and received normal human IgG. The remaining groups of mice (n=2) were used as “naive control” (i.e., no CCI-induction and no administration).

TABLE 12
Treatment Groups

| | | # of | CCI- | | |
|---|---|---|---|---|---|
| Group | Sex | Animal | Induction | Treatment | Administration |
| Sham/Naive | M | 2 | N | — | — |
| CCI-control | M | 6 | Y | Normal | 5 |
| | | | | human IgG | μg/head/week, |
| | | | | antibody | intrathecally |
| CCI- | M | 7 | Y | Anti- | 5 |
| FAM19A1 | | | | FAM19A1 | μg/head/week, |
| | | | | Antibody | intrathecally |

Von Frey test: At days 6, 10, 13, 17 and 20 post chronic constriction of the sciatic nerve, the paw withdrawal frequency was assessed using the Von Frey test (0.16 g). The mice were placed in an apparatus with a wire mesh floor and allowed to stabilize to the environment for about 20 minutes. The filament was applied 10 times to each hind paw with a 10 s interval between each application. Then, the number of paw withdrawal responses was counted and the results of mechanical behavioral testing in the ipsilateral hind paws were expressed as a percent withdrawal response frequency (PWF, %), which represented the percentage of paw withdrawals out of the maximum of 10.

Hargreaves test: At days 6, 10, 13, 17 and 20 post chronic constriction of the sciatic nerve, thermal hypersensitivity was measured as the paw withdrawal response latency (PWL, sec) to a noxious heat stimulus using a plantar testing apparatus (Series 8, Model 390G, IITC Life Science Inc., Woodland Hills, Calif., USA). Before performing the tests, mice were allowed to acclimate to the testing environment, a plastic chamber on an elevated glass plate, for 30 min. The plantar testing apparatus produces a radiant heat source that is positioned under the glass floor beneath the hind paw. A photoelectric cell connected to a digital clock measured the paw withdrawal latency to the radiant heat. The intensity of the light source was adjusted to produce paw withdrawal response latencies of 10-15 s in naive animals. A cut-off time of 20 s was used to protect the animal from excessive tissue damage. The test was duplicated for each hind paw at each time point, and the mean withdrawal latency was then calculated and recorded.

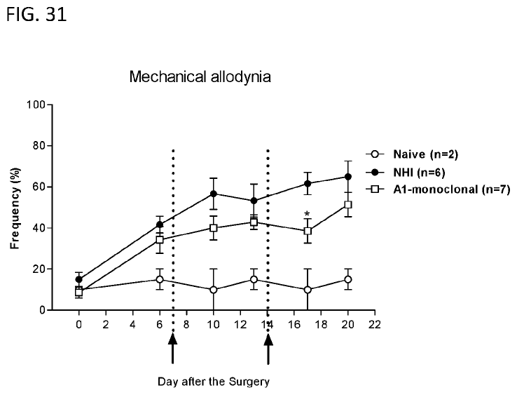

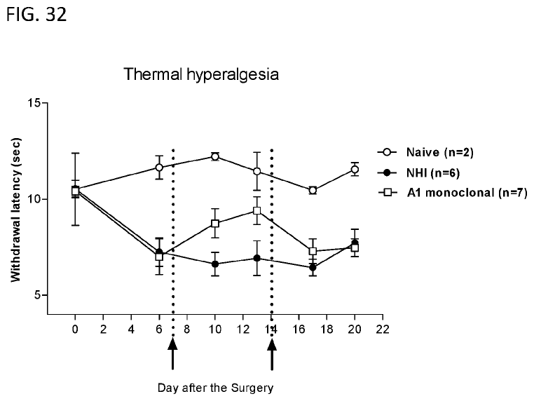

As shown in FIG. 31, CCI induction resulted in the significant increase of paw withdrawal frequency compared to naïve animals (i.e., CCI-induced animals withdrew their paws more often in response to the filament). Once a week (day 7 and day 14 post CCI induction) intrathecal administration of the monoclonal anti-FAM19A1 antibody resulted in significantly decreased paw withdrawal frequency compared to CCI-induced animals treated with the control IgG antibody. Similar results were observed in response to the heat stimulus. CCI-induced animals treated with the monoclonal anti-FAM19A1 antibody had increased withdrawal latency compared to CCI-induced animals treated with the control antibody. FIG. 32.

These results demonstrate the therapeutic effects of the monoclonal anti-FAM19A1 antibody and confirm that neutralizing FAM19A1 activity could alleviate neuropathic pain. Collectively, the results shown in the above Examples demonstrate that the FAM19A1 antagonists disclosed herein could be useful in treating various CNS-related disease and disorders in both male and female subjects.

It is to be appreciated that the Detailed Description including the Summary and Abstract sections is intended to be used to interpret the claims. The Summary and Abstract sections can set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

What is claimed is:

1. An antagonist that specifically binds to a family with sequence similarity 19, member A1 (FAM19A1) (“FAM19A1 antagonist”), for use in therapy.

2. The FAM19A1 antagonist for use of claim 1, wherein the antagonist is capable of treating a disease or disorder in a subject in need thereof.

3. The FAM19A1 antagonist for use of claim 2, wherein the disease or disorder comprises a central nervous system (CNS)-related disease or disorder.

4. The FAM19A1 antagonist for use of claim 3, wherein the CNS-related disease or disorder is associated with an abnormal neural circuit.

5. The FAM19A1 antagonist for use of claim 3 or 4, wherein the CNS-related disease or disorder comprises a mood disorder, psychiatric disorder, or both.

6. The FAM19A1 antagonist for use of any one of claims 3 to 5, wherein the CNS-related disease or disorder comprises an anxiety, depression, post-traumatic stress disorder (PTSD), bipolar disorder, attention deficit/hyperactivity disorder (ADHD), autism, schizophrenia, neuropathic pain, glaucoma, addiction, arachnoid cyst, catalepsy, encephalitis, epilepsy/seizures, Locked-in syndrome, meningitis, migraine, multiple sclerosis, myelopathy, Alzheimer's disease, Huntington's disease, Parkinson's disease, Amyotrophic lateral sclerosis (ALS), Batten disease, Tourette's syndrome, traumatic brain injury, cerebrospinal damage, stroke, tremors (essential or Parkinsonian), dystonia, intellectual disability, brain tumor, or combinations thereof.

7. The FAM19A1 antagonist for use of claim 6, wherein the CNS-related disease or disorder is anxiety, depression, PTSD, or combinations thereof.

8. The FAM19A1 antagonist for use of claim 7, wherein the antagonist is capable of improving one or more symptoms associated with anxiety and/or depression (e.g., increasing the locomotor activity of the subject and/or increasing the subject's ability to respond to an external stress).

9. The FAM19A1 antagonist for use of claim 6, wherein the CNS-related disease or disorder is a glaucoma.

10. The FAM19A1 antagonist for use of claim 9, wherein the antagonist is capable of reducing, ameliorating, or inhibiting inflammation associated with a glaucoma.

11. The FAM19A1 antagonist for use of claim 9 or 10, wherein the antagonist is capable of improving a retinal potential in a retina.

12. The FAM19A1 antagonist for use of any one of claims 9-11, wherein the glaucoma is selected from the group consisting of an open-angle glaucoma, angle-closure glaucoma, normal-tension glaucoma (“NTG”), congenital glaucoma, secondary glaucoma, pigmentary glaucoma, pseudoexfoliative glaucoma, traumatic glaucoma, neovascular glaucoma, irido comeal endothelial syndrome, uveitic glaucoma, and combinations thereof.

13. The FAM19A1 antagonist for use of any one of claims 9-12, wherein the glaucoma is associated with an optic nerve damage, a retinal ganglion cell (“RGC”) loss, a high intraocular pressure (“IOP”), an impaired blood-retina barrier, and/or an increase in a level of microglia activity within a retina and/or optic nerve of the subject.

14. The FAM19A1 antagonist for use of any one of claims 9-13, wherein the glaucoma is caused by a mechanical damage to an optic nerve head and/or an increase in a level of inflammation within a retina and/or an optic nerve of the subject.

15. The FAM19A1 antagonist for use of any one of claims 9-14, wherein the antagonist is capable of delaying an onset of retinal nerve cell degeneration within the subject.

16. The FAM19A1 antagonist for use of any one of claims 9-15, wherein the antagonist is capable of reducing a loss of retinal ganglion cells and/or restoring a retinal ganglion cell number within the retina of the subject.

17. The FAM19A1 antagonist for use of claim 16, wherein the loss of retinal ganglion cells is reduced by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist).

18. The FAM19A1 antagonist for use of claim 16, wherein the retinal ganglion cell number is restored by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist).

19. The FAM19A1 antagonist for use of any one of claims 9-18, wherein the antagonist is capable of protecting nerve connections of an inner plexiform layer of the retina of the subject.

20. The FAM19A1 antagonist for use of claim 6, wherein the CNS-related disease or disorder is a neuropathic pain.

21. The FAM19A1 antagonist for use of claim 20, wherein the antagonist is capable of increasing a threshold or latency to an external stimulus in a subject in need thereof.

22. The FAM19A1 antagonist for use of claim 21, wherein the external stimulus is a mechanical stimulus.

23. The FAM19A1 antagonist for use of claim 21, wherein the external stimulus is a thermal stimulus.

24. The FAM19A1 antagonist for use of any one of claims 21 to 23, wherein the threshold or latency to the external stimulus is increased by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist).

25. The FAM19A1 antagonist for use of any one of claims 20 to 24, wherein the antagonist is capable of increasing or regulating a sensory nerve conduction velocity in a subject in need thereof.

26. The FAM19A1 antagonist for use of claim 25, wherein the sensory nerve conduction velocity is increased by at least about 5%, at least about 10%, at least about 20%, at least about 30%, at least about 40%, at least about 50%, at least about 60%, at least about 70%, at least about 80%, or at least about 90% or more compared to a reference (e.g., corresponding value in a subject who did not receive the FAM19A1 antagonist or corresponding value in the subject prior to administering the FAM19A1 antagonist).

27. The FAM19A1 antagonist for use of any one of claims 20 to 26, wherein the neuropathic pain is a central neuropathic pain or a peripheral neuropathic pain.

28. The FAM19A1 antagonist for use of any one of claims 20 to 27, wherein the neuropathic pain is associated with a physical injury, an infection, diabetes, cancer therapy, alcoholism, amputation, weakness of a muscle in the back, leg, hip, or face, trigeminal neuralgia, multiple sclerosis, shingles, spine surgery, or any combination thereof.

29. The FAM19A1 antagonist for use of any one of claims 20 to 28, wherein the neuropathic pain comprises carpal tunnel syndrome, central pain syndrome, degenerative disk disease, diabetic neuropathy, phantom limb pain, postherpetic neuralgia (shingles), pudendal neuralgia, sciatica, low back pain, trigeminal neuralgia, or any combination thereof.

30. The FAM19A1 antagonist for use of any one of claims 20 to 29, wherein the neuropathic pain is caused by a compression of a nerve.

31. The FAM19A1 antagonist for use of claim 29, wherein the diabetic neuropathy is diabetic peripheral neuropathy.

32. The FAM19A1 antagonist for use of claim 30, wherein the neuropathic pain is sciatica.

33. The FAM19A1 antagonist for use of any one of claims 1 to 32, wherein the antagonist is capable of regulating or improving a central nervous system function in a subject in need thereof.

34. The FAM19A1 antagonist for use of claim 33, wherein the central nervous system function comprises a limbic system related function, olfactory system related function, sensory system related function, visual system related function, or combinations thereof.

35. The FAM19A1 antagonist for use of claim 33 or 34, wherein the antagonist is capable of reducing an expression level of FAM19A1 protein and/or an expression level of FAM19A1 mRNA in a brain region.

36. The FAM19A1 antagonist for use of claim 35, wherein the brain region comprises cerebral cortex, hippocampus, hypothalamus, midbrain, prefrontal cortex, amygdala (e.g., lateral amygdaloid nucleus and basomedial amygdaloid nucleus), piriform cortex, anterior olfactory nucleus, lateral entorhinal cortex, habenula, or combinations thereof.

37. The FAM19A1 antagonist for use of any one of claims 33 to 36, wherein the antagonist is capable of reducing an expression level of FAM19A1 protein and/or an expression level of FAM19A1 mRNA in a retina region.

38. The FAM19A1 antagonist for use of claim 37, wherein the retina region comprises a ganglion cell layer (GCL) or inner plexiform layer (INL).

39. The FAM19A1 antagonist for use of any one of claims 33 to 38, wherein the antagonist is capable of reducing an expression level of FAM19A1 protein and/or an expression level of FAM19A1 mRNA in a spinal cord region.

40. The FAM19A1 antagonist for use of claim 39, wherein the spinal cord region comprises dorsal horn.

41. The FAM19A1 antagonist for use of any one of claims 35 to 40, wherein the expression level of FAM19A1 protein and/or the expression level of FAM19A1 mRNA is reduced by at least about 5%, at least about 10%, at least about 20%, at least about 30%, at least about 40%, at least about 50%, at least about 60%, at least about 70%, at least about 80%, or at least about 90% or more, compared to a reference (e.g., corresponding value in a subject that did not receive the FAM19A1 antagonist or corresponding value in the subject prior to administering the FAM19A1 antagonist).

42. The FAM19A1 antagonist for use of any one of claims 1 to 42, wherein the antagonist is capable of regulating, inducing, or increasing a differentiation of neural stem cells in a subject in need thereof.

43. The FAM19A1 antagonist for use of claim 42, wherein the antagonist is capable of increasing a neurite outgrowth in a differentiated neural stem cell compared to a reference (e.g., corresponding value in a subject who did not receive the FAM19A1 antagonist or corresponding value in the subject prior to administering the FAM19A1 antagonist).

44. The FAM19A1 antagonist for use of claim 43, wherein the neurite outgrowth is increased by at least about 5%, at least about 10%, at least about 20%, at least about 30%, at least about 40%, at least about 50%, at least about 60%, at least about 70%, at least about 80%, or at least about 90% or more compared to the reference.

45. A method of diagnosing an abnormality in a central nervous system (CNS) function in a subject in need thereof comprising contacting a FAM19A1 antagonist with a sample of the subject and measuring a FAM19A1 protein level or a FAM19A1 mRNA level in the sample.

46. A method of identifying a subject with an abnormality in a central nervous system (CNS) function comprising contacting a FAM19A1 antagonist with a sample of the subject and measuring a FAM19A1 protein level or a FAM19A1 mRNA level in the sample.

47. The method of claim 45 or 46, wherein the contacting and the measuring is performed in vitro.

48. The method of any one of claims 45 to 47, wherein the CNS function comprises a limbic system related function, olfactory system related function, sensory system related function, visual system related function, or combinations thereof.

49. The method of any one of claims 45 to 48, wherein the abnormality in a CNS function is associated with an abnormal neural circuit.

50. The method of any one of claims 45 to 49, wherein the abnormality in a CNS function is associated with a CNS-related disease or disorder.

51. The method of claim 50, wherein the CNS-related disease or disorder comprises a mood disorder, psychiatric disorder, or both.

52. The method of claim 50 or 51, wherein the CNS-related disease or disorder comprises an anxiety, depression, post-traumatic stress disorder (PTSD), bipolar disorder, attention deficit/hyperactivity disorder (ADHD), autism, schizophrenia, neuropathic pain, glaucoma, addiction, arachnoid cyst, catalepsy, encephalitis, epilepsy/seizures, Locked-in syndrome, meningitis, migraine, multiple sclerosis, myelopathy, Alzheimer's disease, Huntington's disease, Parkinson's disease, Amyotrophic lateral sclerosis (ALS), Batten disease, Tourette's syndrome, traumatic brain injury, cerebrospinal damage, stroke, tremors (essential or Parkinsonian), dystonia, intellectual disability, brain tumor, or combinations thereof.

53. The method of claim 52, wherein the CNS-related disease or disorder is anxiety, depression, PTSD, or combinations thereof.

54. The method of claim 52, wherein the CNS-related disease or disorder is glaucoma, neuropathic pain, or both.

55. The method of any one of claims 45 to 54, wherein the abnormality in a central nervous system function is associated with an increase in the FAM19A1 protein level and/or in the FAM19A1 mRNA level in the sample compared to a reference (e.g., corresponding value in a sample of a subject who does not suffer from an abnormality in a central nervous system function, e.g., a healthy subject).

56. The method of claim 56, wherein the FAM19A1 protein level and/or FAM19A1 mRNA level is increased by at least about 5%, at least about 10%, at least about 20%, at least about 30%, at least about 40%, at least about 50%, at least about 60%, at least about 70%, at least about 80%, or at least about 90% or more, compared to the reference.

57. The method of any one of claims 45 to 54, wherein the abnormality in a central nervous system function is associated with a decrease in the FAM19A1 protein level and/or in the FAM19A1 mRNA level in the sample compared to a reference (e.g., corresponding value in a sample of a subject who does not suffer from an abnormality in a central nervous system function, e.g., a healthy subject).

58. The method of claim 57, wherein the FAM19A1 protein level and/or FAM19A1 mRNA level is decreased by at least about 5%, at least about 10%, at least about 20%, at least about 30%, at least about 40%, at least about 50%, at least about 60%, at least about 70%, at least about 80%, or at least about 90% or more, compared to the reference.

59. The method of any one of claims 45 to 58, wherein the FAM19A1 protein level is measured by an immunohistochemistry, Western blot, radioimmunoassay, enzyme linked immunosorbent assay (ELISA), radioimmunodiffusion, immunoprecipitation assay, Ouchterlony immunodiffusion method, rocket immunoelectrophoresis, tissue immunostaining method, complement fixation assay, FACS, protein chip, or combinations thereof.

60. The method of any one of claims 45 to 58, wherein the FAM19A1 mRNA level is measured by a reverse transcription polymerase chain reaction (RT-PCR), a real time polymerase chain reaction, a Northern blot, or combinations thereof.

61. The method of any one of claims 45 to 60, wherein the sample comprises a tissue, cell, blood, serum, plasma, saliva, urine, cerebral spinal fluid (CSF), or combinations thereof.

62. The method of any one of claims 45 to 56 and 59 to 61, further comprising administering a FAM19A1 antagonist to the subject if the FAM19A1 protein level and/or FAM19A1 mRNA level is increased compared to the reference.

63. The method of any one of claims 45 to 54 and 57 to 61, further comprising administering an agonist against FAM19A1 (“FAM19A1 agonist”) if the FAM19A1 protein level and/or FAM19A1 mRNA level is decreased compared to the reference.

64. The method of claim 63, wherein the FAM19A1 agonist is a FAM19A1 protein.

65. The FAM19A5 antagonist for use or the method of any one of claims 1 to 64, wherein the FAM19A1 antagonist is an antisense oligonucleotide, siRNA, shRNA, miRNA, dsRNA, aptamer, PNA that specifically targets FAM19A1, or a vector including the same.

66. The FAM19A5 antagonist for use or the method of any one of claims 1 to 64, wherein the FAM19A1 antagonist is an anti-FAM19A1 antibody, a polynucleotide encoding the anti-FAM19A1 antibody, a vector comprising the polynucleotide thereof, a cell comprising the polynucleotide thereof, or any combination thereof.

67. The FAM19A5 antagonist for use or the method of claim 66, wherein the FAM19A1 antagonist is an anti-FAM19A1 antibody.

68. The FAM19A5 antagonist for use or the method of any one of claims 2 to 67, wherein the subject is a male.

69. An anti-FAM19A1 antibody or antigen-binding fragment thereof (“an anti-FAM19A1 antibody”) that exhibits a property selected from:
(a) binds to soluble human FAM19A1 with a K_{D} of 10 nM or less as measured by ELISA;
(b) binds to membrane bound human FAM19A1 with a K_{D} of 10 nM or less as measured by ELISA; or
(c) both (a) and (b)

70. The anti-FAM19A1 antibody of claim 69, which cross-competes for binding to a human FAM19A1 epitope with a reference antibody comprising a heavy chain CDR1, CDR2, and CDR3 and a light chain CDR1, CDR2, and CDR3,
(i) wherein the heavy chain CDR1 comprises the amino acid sequence set forth in SEQ ID NO: 10, the heavy chain CDR2 comprises the amino acid sequence set forth in SEQ ID NO: 11, the heavy chain CDR3 comprises the amino acid sequence set forth in SEQ ID NO: 12, the light chain CDR1 comprises the amino acid sequence set forth in SEQ ID NO: 13, the light chain CDR2 comprises the amino acid sequence set forth in SEQ ID NO: 14, and the light chain CDR3 comprises the amino acid sequence set forth in SEQ ID NO: 15;
(ii) wherein the heavy chain CDR1 comprises the amino acid sequence set forth in SEQ ID NO: 4, the heavy chain CDR2 comprises the amino acid sequence set forth in SEQ ID NO: 5, the heavy chain CDR3 comprises the amino acid sequence set forth in SEQ ID NO: 6, the light chain CDR1 comprises the amino acid sequence set forth in SEQ ID NO: 7, the light chain CDR2 comprises the amino acid sequence set forth in SEQ ID NO: 8, and the light chain CDR3 comprises the amino acid sequence set forth in SEQ ID NO: 9;
(iii) wherein the heavy chain CDR1 comprises the amino acid sequence set forth in SEQ ID NO: 16, the heavy chain CDR2 comprises the amino acid sequence set forth in SEQ ID NO: 17, the heavy chain CDR3 comprises the amino acid sequence set forth in SEQ ID NO: 18, the light chain CDR1 comprises the amino acid sequence set forth in SEQ ID NO: 19, the light chain CDR2 comprises the amino acid sequence set forth in SEQ ID NO: 20, and the light chain CDR3 comprises the amino acid sequence set forth in SEQ ID NO: 21; or
(iv) wherein the heavy chain CDR1 comprises the amino acid sequence set forth in SEQ ID NO: 22, the heavy chain CDR2 comprises the amino acid sequence set forth in SEQ ID NO: 23, the heavy chain CDR3 comprises the amino acid sequence set forth in SEQ ID NO: 24, the light chain CDR1 comprises the amino acid sequence set forth in SEQ ID NO: 25, the light chain CDR2 comprises the amino acid sequence set forth in SEQ ID NO: 26, and the light chain CDR3 comprises the amino acid sequence set forth in SEQ ID NO: 27.

71. The anti-FAM19A1 antibody of claim 69 or 70, which binds to the same FAM19A1 epitope as a reference antibody comprising a heavy chain CDR1, CDR2, and CDR3 and a light chain CDR1, CDR2, and CDR3,
(i) wherein the heavy chain CDR1 comprises the amino acid sequence set forth in SEQ ID NO: 10, the heavy chain CDR2 comprises the amino acid sequence set forth in SEQ ID NO: 11, the heavy chain CDR3 comprises the amino acid sequence set forth in SEQ ID NO: 12, the light chain CDR1 comprises the amino acid sequence set forth in SEQ ID NO: 13, the light chain CDR2 comprises the amino acid sequence set forth in SEQ ID NO: 14, and the light chain CDR3 comprises the amino acid sequence set forth in SEQ ID NO: 15;
(ii) wherein the heavy chain CDR1 comprises the amino acid sequence set forth in SEQ ID NO: 4, the heavy chain CDR2 comprises the amino acid sequence set forth in SEQ ID NO: 5, the heavy chain CDR3 comprises the amino acid sequence set forth in SEQ ID NO: 6, the light chain CDR1 comprises the amino acid sequence set forth in SEQ ID NO: 7, the light chain CDR2 comprises the amino acid sequence set forth in SEQ ID NO: 8, and the light chain CDR3 comprises the amino acid sequence set forth in SEQ ID NO: 9;
(iii) wherein the heavy chain CDR1 comprises the amino acid sequence set forth in SEQ ID NO: 16, the heavy chain CDR2 comprises the amino acid sequence set forth in SEQ ID NO: 17, the heavy chain CDR3 comprises the amino acid sequence set forth in SEQ ID NO: 18, the light chain CDR1 comprises the amino acid sequence set forth in SEQ ID NO: 19, the light chain CDR2 comprises the amino acid sequence set forth in SEQ ID NO: 20, and the light chain CDR3 comprises the amino acid sequence set forth in SEQ ID NO: 21; or
(iv) wherein the heavy chain CDR1 comprises the amino acid sequence set forth in SEQ ID NO: 22, the heavy chain CDR2 comprises the amino acid sequence set forth in SEQ ID NO: 23, the heavy chain CDR3 comprises the amino acid sequence set forth in SEQ ID NO: 24, the light chain CDR1 comprises the amino acid sequence set forth in SEQ ID NO: 25, the light chain CDR2 comprises the amino acid sequence set forth in SEQ ID NO: 26, and the light chain CDR3 comprises the amino acid sequence set forth in SEQ ID NO: 27.

72. The anti-FAM19A1 antibody of any one of claims 69 to 71, which binds to at least one epitope selected from the group consisting of D112, M117, A119, T120, N122, and combinations thereof.

73. The anti-FAM19A1 antibody of any one of claims 69 to 72, which comprises a heavy chain CDR1, CDR2, and CDR3 and a light chain CDR1, CDR2, and CDR3, wherein the heavy chain CDR3 comprises the amino acid sequence set forth in SEQ ID NO: 12, 6, 18, or 24.

74. The anti-FAM19A1 antibody of claim 73, wherein the heavy chain CDR1 comprises the amino acid sequence set forth in SEQ ID NO: 10, 4, 16, or 22.

75. The anti-FAM19A1 antibody of claim 73 or 74, wherein the heavy chain CDR2 comprises the amino acid sequence set forth in SEQ ID NO: 11, 5, 17, or 23.

76. The anti-FAM19A1 antibody of any one of claims 73 to 75, wherein the light chain CDR1 comprises the amino acid sequence set forth in SEQ ID NO: 13, 7, 19, or 25.

77. The anti-FAM19A1 antibody of any one of claims 73 to 76, wherein the light chain CDR2 comprises the amino acid sequence set forth in SEQ ID NO: 14, 8, 20, or 26.

78. The anti-FAM19A1 antibody of any one of claims 73 to 77, wherein the light chain CDR3 comprises the amino acid sequence set forth in SEQ ID NO: 15, 9, 21, or 27.

79. The anti-FAM19A1 antibody of any one of claims 69 to 72, comprising a heavy chain CDR1, CDR2, and CDR3 and a light chain CDR1, CDR2, and CDR3, wherein
(i) the heavy chain CDR1, CDR2, and CDR3 comprises the amino acid sequence set forth in SEQ ID NOs: 10-12, respectively, and the light chain CDR1, CDR2, and CDR3 comprises the amino acid sequence set forth in SEQ ID NOs: 13-15, respectively;
(ii) the heavy chain CDR1, CDR2, and CDR3 comprises the amino acid sequence set forth in SEQ ID NOs: 4-6, respectively, and the light chain CDR1, CDR2, and CDR3 comprises the amino acid sequence set forth in SEQ ID NOs: 7-9, respectively;
(iii) the heavy chain CDR1, CDR2, and CDR3 comprises the amino acid sequence set forth in SEQ ID NOs: 16-18, respectively, and the light chain CDR1, CDR2, and CDR3 comprises the amino acid sequence set forth in SEQ ID NOs: 19-21, respectively; or
(iv) the heavy chain CDR1, CDR2, and CDR3 comprises the amino acid sequence set forth in SEQ ID NOs: 22-24, respectively, and the light chain CDR1, CDR2, and CDR3 comprises the amino acid sequence set forth in SEQ ID NOs: 25-27, respectively.

80. The anti-FAM19A1 antibody of any one of claims 69 to 79, comprising a heavy chain variable domain comprising the amino acid sequence set forth in SEQ ID NO: 30, 28, 32, or 34 and/or a light chain variable domain comprising the amino acid sequence set forth in SEQ ID NO: 31, 29, 33, or 35.

81. The anti-FAM19A1 antibody of claim 80, comprising a heavy chain variable domain comprising the amino acid sequence set forth in SEQ ID NO: 30 and a light chain variable domain comprising the amino acid sequence set forth in SEQ ID NO: 31.

82. The anti-FAM19A1 antibody of claim 80, comprising a heavy chain variable domain comprising the amino acid sequence set forth in SEQ ID NO: 28 and a light chain variable domain comprising the amino acid sequence set forth in SEQ ID NO: 29.

83. The anti-FAM19A1 antibody of claim 80, comprising a heavy chain variable domain comprising the amino acid sequence set forth in SEQ ID NO: 32 and a light chain variable domain comprising the amino acid sequence set forth in SEQ ID NO: 33.

84. The anti-FAM19A1 antibody of claim 80, comprising a heavy chain variable domain comprising the amino acid sequence set forth in SEQ ID NO: 34 and a light chain variable domain comprising the amino acid sequence set forth in SEQ ID NO: 35.

85. The anti-FAM19A1 antibody of any one of claims 69 to 72, comprising a heavy chain variable region and a light chain variable region, wherein the heavy chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 30, 28, 32, or 34; and/or wherein the light chain variable region comprises an amino acid sequence which is at least about 80%, at least about 85%, at least about 90%, at least about 95%, at least about 96%, at least about 97%, at least about 98%, at least about 99%, or about 100% identical to the amino acid sequence set forth in SEQ ID NO: 31, 29, 33, or 35.

86. The anti-FAM19A1 antibody of any of claims 69 to 73, which is a chimeric antibody, a human antibody, or a humanized antibody.

87. The anti-FAM19A1 antibody of any one of claims 69 to 86, comprising a Fab, a Fab′, a F(ab′)2, a Fv, or a single chain Fv (scFv).

88. The anti-FAM19A1 antibody of any one of claims 69 to 87, which is selected from the group consisting of an IgG1, an IgG2, an IgG3, an IgG4, a variant thereof, and any combination thereof.

89. The anti-FAM19A1 antibody of claim 88, which is an IgG1 antibody.

90. The anti-FAM19A1 antibody of any one of claims 69 to 89 further comprising a constant region without a Fc function.

91. The anti-FAM19A1 antibody of any one of claims 69 to 90, which is linked to an agent, thereby forming an immunoconjugate.

92. The anti-FAM19A1 antibody of any one of claims 69 to 91, which is formulated with a pharmaceutically acceptable carrier.

93. The FAM19A5 antagonist for use or the method of any one of claims 1 to 68, wherein the FAM19A1 antagonist is the anti-FAM19A1 antibody of any one of claims 69 to 92.

94. The FAM19A5 antagonist for use or the method of any one of claims 1 to 68 and 93, wherein the FAM19A1 antagonist is capable of being administered intravenously, orally, parenterally, intrathecally, intra-cerebroventricularly, pulmonarily, intramuscularly, subcutaneously, intravitreally, or intraventricularly.

95. The FAM19A5 antagonist for use or the method of any one of claims 1 to 68, 93, and 94, wherein the subject is a human.

96. A nucleic acid comprising a nucleotide sequence encoding the anti-FAM19A1 antibody of any one of claims 69 to 92.

97. A vector comprising the nucleic acid of claim 96 and one or more promoters operably linked to the nucleic acid.

98. A cell comprising the nucleic acid of claim 96 or the vector of claim 97.

99. A composition comprising the anti-FAM19A1 antibody of any one of claims 69 to 92, and a carrier.

100. A kit comprising the anti-FAM19A1 antibody of any one of claims 69 to 92, and instructions for use.

101. A method of producing an anti-FAM19A1 antibody comprising culturing the cell of claim 98 under suitable condition and isolating the anti-FAM19A1 antibody.
